(19) United States
(12) Patent Application Publication
(10) Pub. No.: US 2023/0035763 A1
(43) Pub. Date: Feb. 2, 2023

(54) METHODS OF TREATING CANCER USING TUBULIN BINDING AGENTS

(71) Applicant: BeyondSpring Pharnaceuticals, Inc., New York, NY (US)

(72) Inventors: James R. Tonra, New York, NY (US); Lan Huang, New York, NY (US)

(21) Appl. No.: 17/293,418

(22) PCT Filed: Nov. 12, 2019
(86) PCT No.: PCT/US2019/061004

§ 371 Date: May 12, 2021

Related U.S. Application Data
(60) Provisional application No. 62/767,376, filed on Nov. 14, 2018.

(51) Int. Cl. C12Q 1/6886; A61K 31/496; A61K 31/337; G16B 40/20; G16B 25/10; G16H 20/40; G16H 50/20

(52) U.S. Cl. CPC C12Q 1/6886; A61K 31/496; A61K 31/337; G16B 40/20; C12Q 2600/106; G16H 20/40; G16H 50/20; C12Q 2600/158; G16B 25/10

(57) ABSTRACT

Described herein includes a method of treating a cancer. The method includes selecting a patient responsive to treatment with a tubulin binding agent by determining an expression level of a biomarker panel; and administering the tubulin binding agent to the selected patient. The biomarker can be one or more probesets listed in Tables 1-2 or 4 or the gene expressions identifiable using the probesets listed in Tables 1-2 or 4.

### CROSS-REFERENCE TO RELATED APPLICATIONS

This application is a national phase application of international PCT Application No. PCT/US2019/061004, filed on Nov. 12, 2019, which claims benefit of Provisional Application No. 62/767,376, filed on Nov. 14, 2018, which is hereby incorporated by reference in its entirety.

### BACKGROUND OF THE INVENTION

### Field of the Invention

The present invention relates to methods of selecting patients for cancer treatment and administering chemotherapeutic agents to selected patients.

### Description of the Related Art

Traditional chemotherapy treatment paradigms used by physicians have been to prescribe a drug therapy that results in the highest success rate possible for treating a disease. Alternative drug therapies are then prescribed if the first is ineffective. The risk of non-responsiveness to chemotherapy agents is often accepted. However, because the effectiveness of chemotherapy often decreases with each subsequent therapy, selecting the most effective first treatment or selecting a patient that responds to the specific cancer drug is critical in leading to the greatest long term benefit for the greatest number of patients. Therefore, there exists a heightened need to choose an initial drug that will be the most effective against that particular patient's disease.

### SUMMARY OF THE INVENTION

Some embodiments relate to a method of treating a cancer, the method comprising selecting a subject responsive to treatment with a tubulin binding agent by determining an expression level of one or more biomarkers; and administering an effective amount of the tubulin binding agent to the selected subject.

Some embodiments relate to a method of generating a predictive model for assessing a subject's response to a chemotherapy drug, the method comprising: obtaining expression levels of a plurality of biomarkers in at least one cancer cell line; determining an inhibition activity of the chemotherapy drug on the plurality of cancer cell lines; determining a relationship between the expression levels of the plurality of biomarkers and the inhibition activity of the chemotherapy drug; and generating the predictive model based on the relationship between the expression levels of the plurality of biomarkers and the inhibition concentration of the chemotherapy drug.

### BRIEF DESCRIPTION OF THE DRAWINGS

FIG. 1 is a scatter plot matrix showing the top 10 of 200 probeset values after Bootstrap Forest Partitioning analysis (x-axis) versus tubulin targeted agent anticancer cell efficacy (IC₇₀)

FIG. 2 shows a mathematical model calculating the neural probability function (3 hidden nodes, range from 0-1, with 1 being the highest probability for plinabulin active), using CALD1, SECISBP2L, UBXN8, AUP1, and CDCA5 HIT probeset mRNA Expression Values.

FIG. 3 shows a model for calculating the neural probability function (3 hidden nodes, range from 0-1, with 1 being the highest probability for plinabulin active), using CALD1, SECISBP2L, UBXN8, AUP1, CDCA5, TM9SF3, 232522_at, LGR5, 214862_x_at, and FAM98B.

FIG. 4 shows a model for calculating the neural probability function (1 hidden node, Range from 0-1, With 1 Being the Highest Probability for Docetaxel Active), using CALD1, SECISBP2L, UBXN8, AUP1, and CDCA5 HIT Probeset mRNA Expression Values.

FIG. 5 shows a model for calculating the neural probability function (3 hidden nodes, range from 0-1, with 1 being the highest probability for plinabulin active), using CALD1, UBXN8, and CDCA5 HIT Probeset mRNA Expression Values

FIG. 6 is a 3-Dimensional Plot of Neural Model Derived Probability from FIG. 5, Versus Actual IC₇₀ Determined Plinabulin Activity in 43 Cell Lines.

FIG. 7 shows a model for calculating the neural probability function (1 hidden node, Range from 0-1, With 1 Being the Highest Probability for Docetaxel Active), Using CALD1, SECISBP2L, UBXN8, and AUP1 HIT Probeset mRNA Expression Values.

FIG. 8 shows a binomial logistic probability function (range from 0-1, with 1 being the highest probability for plinabulin inactive), using CALD1, SECISBP2L, UBXN8, AUP1, and CDCA5 HIT Probeset mRNA expression values.

FIG. 9 shows a 3-dimensional plot of binomial logistic regression model derived probability from FIG. 8, versus IC₇₀ determined Plinabulin activity (prob[inactive] can range from 0-1) in 43 cell lines.

### DETAILED DESCRIPTION OF THE PREFERRED EMBODIMENT

Disclosed herein are methods of selecting patients suitable for treatment using tubulin binding agents. One embodiment is the stratification of patient's response to certain chemotherapeutic drugs and selection of patients for cancer therapeutic drugs and thus guide patient treatment selection. Another embodiment is the stratification of cancer patients into those that respond and those that do not respond to chemotherapy such as tubulin binding agent treatment. The methods described herein can guide selecting patients prior to or during the chemotherapy treatment. The test described herein can be used as a prognostic indicator for certain cancers including central nervous system (CNS) lymphoma, lung cancer, breast cancer, ovarian cancer, and prostate cancer.

Tubulin binding drugs are approved for the treatment of many cancer types. High expression of transporter proteins that bind some anticancer tubulin targeted agents that have entered tumor cells, pump them outside of the cell (extracellular), enabling these cancer cells to resist the cytotoxic effects of these agents. Patients of certain approved cancer types that are prescribed taxanes alone or in combination with other chemotherapies have their disease evaluated at scheduled intervals to evaluate tumor progression. If tumor progression is detected, months after starting therapy, an alternative therapy, if available, is selected. However such methods are not commonly utilized. A method of confidently selecting patients with cancer cells that are insensitive to taxanes would be of great value by allowing these patients to be prescribed another therapy with greater potential to kill cancer cells, even if they have a cancer type approved for taxane therapy. Moreover, this method could be utilized in the future to select new responsive cancer types, and to select patients independent of cancer type that may be especially sensitive to taxanes. In some embodiments, the tubulin binding agent is Plinabulin. In some embodiments, the tubulin binding agent is a taxane. In some embodiments, the tubulin binding agent is a docetaxel. In some embodiments, the tubulin binding agent is a paclitaxel. In some embodiments, the tubulin binding agent is an agent that binds to a Vinca site. In some embodiments, the tubulin binding agent is vinblastine or vincristine.

Plinabulin is a tubulin targeted agent that binds near the colchicine site in β-tubulin and is being tested in a Phase 3 clinical study for the treatment of non-small cell lung cancer. The colchicine site is distinct from the binding site of taxanes (e.g. Paclitaxel and docetaxel), and binding site and other differences between tubulin targeted agents are often associated with differing effects on biological functions, disease outcomes and safety profiles. Additional indications are being considered for plinabulin so a model for selecting especially responsive patients would be of significant value. As a first step towards building this model, the in vitro activity of Plinabulin against 43 human cancer cell lines (breast, lung, prostate, ovarian or CNS), previously characterized for mRNA expression with the Affymetrix HGU133 Plus 2.0 array, was evaluated. Although screening for in vitro anticancer activity is typically performed with constant treatment of the agent for 48-72 hours, cells were treated for only 24 hours with plinabulin and then cultured for another 48 hours without plinabulin.

Typically anticancer activity is judged at the 50% effect level (50% reduction in viable tumor cells), but viable cell concentration are quantified here with a Cell Titer-Blue Assay to find the concentration causing a 70% reduction in the quantity of viable tumor cells (IC₇₀). With these methods, cell lines can be separated into plinabulin Active (21 cell lines with IC₇₀<1.0 μM) and Inactive (91% with IC₇₀>9.5 μM) categories, with very few cells having a plinabulin IC₇₀ between 1 and 9.5 μM. Utilizing JMP 14.1 Statistical software, log 2 transformed Affymetrix gene probeset signal values, preprocessed with the GeneChip robust multi-array average analysis algorithm, can be ranked for predicting plinabulin activity utilizing two “HIT” probeset identification strategies. Through these efforts, 56 HIT probesets with predictive power can be identified (one per gene) that also exhibit differential expression in plinabulin responding versus non-responding cell lines (p<0.01, t-test), and therefore the potential to predict plinabulin potency. For probesets with gene annotation, only the probeset for each gene with the highest Jetset score is utilized. From the HIT predictor gene probesets, multiple one-layer Tan H multimode fit neural network models were constructed to identify plinabulin responding cell lines with confidence, in both a training (⅔ of models tested) and validation set. Similar results were obtained utilizing a non-neural binomial logistic model. The power of these novel algorithms to predict potent anticancer activity, utilizing just 3-10 mRNA measurements was striking and unexpected.

Some of the same probesets used to develop predictive algorithms for plinabulin activity showed differential expression in docetaxel responding versus non-responding tumor cell lines and can be successfully utilized in developing predictive models of docetaxel anticancer cell activity. This indicates that the overall strategy and identified probesets/gene expression evaluations, and predictive mathematical algorithms developed with a combination of these probeset evaluations, may be applicable for predicting response across tubulin targeted agents.

Various tubulin targeted agents (a taxane and an agent that binds near the colchicine binding pocket) can be used to discover genes/probesets with expression levels that correlate with tubulin targeted agent anticancer potency, and to discover predictive algorithms through novel analytical strategies. These measurements, analytical strategies and algorithms can be used in selecting cancer patients with tumors cells that are particularly susceptible to the direct cytotoxic effects of plinabulin and other tubulin binding agents.

The methods described herein can help increase the efficacy of chemotherapy (i.e., tubulin binding agents) in patients by incorporating molecular parameters into clinical therapeutic decisions. Pharmacogenetics/genomics is the study of genetic/genomic factors involved in an individuals' response to a foreign compound or drug. Methods of determining the patient's response based on the patient's genetic factors allows for the selection of effective agents (e.g., drugs) for prophylactic or therapeutic treatments. Such pharmacogenomics can further be used to determine appropriate dosages and therapeutic regimens. Accordingly, the level of expression of a biomarker of the invention in an individual can be determined to thereby select appropriate agent(s) for therapeutic or prophylactic treatment of the individual.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from or developing a disease or condition.

### Method of Treatment

Some embodiments relate to a method of treating a cancer, comprising selecting a subject responsive to treatment with a tubulin binding agent by determining expression levels of one or more biomarker; and administering the tubulin binding agent to the selected subject. In some embodiments, the method includes using an expression score to classify a subject as responsive or non-responsive to a chemotherapy and/or having a good or poor clinical prognosis.

The biomarker can include a gene, an mRNA, cDNA, an antisense transcript, a miRNA, a polypeptide, a protein, a protein fragment, or any other nucleic acid sequence or polypeptide sequence. In some embodiments, the biomarkers are RNA. In some embodiments, the biomarkers are mRNA. In some embodiments, biomarker suitable for use can include DNA, RNA, and proteins. The biomarkers are isolated from a subject sample and their expression levels determined to derive a set of expression profiles for each sample analyzed in the subject sample set.

Measuring mRNA in a biological sample may be used as a surrogate for detection of the level of the corresponding protein and gene in the biological sample. Thus, any of the biomarkers described herein can also be detected by detecting the appropriate RNA. Methods of biomarker expression profiling include, but are not limited to probeset, quantitative PCR, NGS, northern blots, southern blots, microarrays, SAGE, immunoassays (ELISA, EIA, agglutination, nephelometry, turbidimetry, Western blot, immunoprecipitation, immunocytochemistry, flow cytometry, Luminex assay), and mass spectrometry. The overall expression data for a given sample may be normalized using methods known to those skilled in the art in order to correct for differing amounts of starting material, varying efficiencies of the extraction and amplification reactions.

In one exemplary embodiment, the biomarkers is selected from the one or more genes selected from CALD1, UBXN8, CDCA5, ERI1, SEC14L1P1, SECISBP2L/SLAN, WDR20, LGR5, ADIPOR2, RUFY2, COL5A2, YTHDC2, RPL12, MTMR9, TM9SF3, CALB2, WDR92, DGUOK, CTNNB1, FKBP4, BRPF3, DENND2D, TMEM47, RPS19, AUP1, ZFX, MRPL30, TRAK1, RCCD1, ZMAT3, GEMIN7, ZNF106, GLT8D1, CASC4, FAM98B, NME1-NME2, HOOK3, CSTF3, ACTR3, RPL38, PLOD1, MARS, ZNF441, RELB, NLE1, MRPS23, and any combinations thereof. In some embodiments, the biomarker is selected from the group consisting of CALD1, SECISBP2L, UBXN8, AUP1, CDCA5, TM9SF3, LGR5, FAM98B, and combinations thereof. In some embodiments, the biomarker is selected from the group consisting of CALD1, SECISBP2L, UBXN8, AUP1, CDCA5, and any combinations thereof. In some embodiments, the biomarker is selected from the group consisting of CALD1, UBXN8, AUP1, CDCA5, and any combinations thereof. In some embodiments, the biomarker is selected from the group consisting of CALD1, SECISBP2L, UBXN8, AUP1, and any combinations thereof.

The expression profile from the sample set are then analyzed using a mathematical model. Different predictive mathematical models may be applied and include, but are not limited to, multiple one-layer Tan H multimode fit neural network models, non-neural ordinal logistic model, and combinations thereof. In some embodiments, the mathematical model identifies or defines a variable, such as a weight, for each identified biomarker. In certain embodiments, the mathematical model defines a decision function. The decision function may further define a threshold score which separates the sample set into two groups as responsive or non-responsive to a chemotherapy.

In some embodiments, the method described herein is the identification of patients with good and poor prognosis. By examining the expression of the identified biomarkers in a tumor, it is possible to determine the likely clinical outcomes of a patient. By examining the expression of a collection of biomarkers, it is therefore possible to identify those patients in most need of more aggressive therapeutic regimens and likewise eliminate unnecessary therapeutic treatments or those unlikely to significantly improve a patient's clinical outcome.

In some embodiments, the method described here in includes determining an expression score or threshold score using the determined expression level of the one or more biomarkers. The expression score or threshold score is derived by obtaining an expression level based on the samples taken from the subject. The samples may originate from the same sample tissue type or different tissue types. In some embodiments, the expression profile comprises a set of values representing the expression levels for each biomarker analyzed from a given sample.

In other embodiments, the expression score disclosed herein is the stratification of response to, and selection of subject for therapeutic drug such as tubulin binding agents. By examining the expression of the identified biomarkers in a tumor or cancer, it is possible to determine whether the chemotherapeutic agent(s) will be most likely to reduce the growth rate of a cancer. It is also possible to determine whether the chemotherapeutic agent(s) will be the least likely to reduce the growth rate of a cancer. By examining the expression of identified biomarkers, it is therefore possible to eliminate ineffective or inappropriate therapeutic agents. Importantly, in certain embodiments, these determinations can be made on a patient-by-patient basis or on an agent-by-agent basis. Thus, one can determine whether or not a particular therapeutic regimen is likely to benefit a particular patient or type of patient, and/or whether a particular regimen should be continued. The present invention provides a test that can guide therapy selection as well as selecting patient groups for enrichment strategies during clinical trial evaluation of novel therapeutics. For example, when evaluating chemotherapeutic agent(s) or treatment regime, the expression signatures and methods disclosed herein may be used to select individuals for clinical trials that have cancer subtypes that are responsive to anti-angiogenic agents.

In some embodiments, the method described herein can include obtaining a test sample from the subject; determining an expression score by using the determined expression level of the one or more biomarkers; and classifying the subject as responsive or non-responsive to the tubulin binding agent treatment based on the expression score.

In some embodiments, classifying the subject comprises classifying the subject as responsive or nonresponsive by comparing the expression score with a reference. In some embodiments, classifying the subject comprises classifying the subject as non-responsive when the expression score is lower than the reference. In some embodiments, classifying the subject comprises classifying the subject as non-responsive when the expression score is greater than the reference. In some embodiments, classifying the subject comprises classifying the subject as responsive when the expression score is greater than the reference. In some embodiments, classifying the subject comprises classifying the subject as responsive when the expression score is lower than the reference.

In some embodiments, classifying the subject comprises classifying the subject as responsive when the expression score is closer to a predetermined responsive score than to a predetermined nonresponsive score. In some embodiments, classifying the subject comprises classifying the subject as nonresponsive when the expression score is closer to a predetermined nonresponsive score than to a predetermined responsive score. In some embodiments, classifying the subject as responsive or nonresponsive comprises predetermining a responsive score as indicative of the high probability of patient's response to treatment and predetermining a nonresponsive score as indicative of the low probability of the patient's response to treatment. In some embodiments, classifying the subject as responsive or nonresponsive further comprises comparing the expression score with the predetermined responsive score and nonresponsive score, determining whether the expression score is closer to the predetermined responsive score or nonresponsive score. In some embodiments, the predetermined responsive or nonresponsive score is indicative of the chemotherapy drug's effectiveness in inhibiting or reducing the cancer/tumor cells. In some embodiments, the predetermined responsive or nonresponsive score is indicative of the inhibition activity of the chemotherapy drug. In some embodiments, the predetermined responsive or nonresponsive score is indicative of the IC₇₀ of the chemotherapy drug. In some embodiments, the predetermined responsive or nonresponsive score is indicative of the IC₅₀ of the chemotherapy drug. In some embodiments, the predetermined responsive score is indicative of a IC₇₀ of lower than about 50, 40, 30, 20, 15, 10, 9, 8, 7, 6, 5, 4, 3, 2, 1, 0.5, or 0.1 μM when the chemotherapy drug is tested on the cancer cell line(s). In some embodiments, the predetermined responsive score is indicative of a IC₇₀ of lower than 1 μM when the chemotherapy drug is tested on the cancer cell line(s). In some embodiments, the predetermined responsive score is indicative of a IC₅₀ of lower than about 50, 40, 30, 20, 15, 10, 9, 8, 7, 6, 5, 4, 3, 2, 1 μM when the chemotherapy drug is tested on the cancer cell line(s). In some embodiments, the predetermined nonresponsive score is indicative of a IC₇₀ of greater than 1 μM when the chemotherapy drug is tested on the cancer cell line(s). In some embodiments, the predetermined nonresponsive score is indicative of a IC₇₀ of greater than about 0.5, 1, 2, 3, 4, 5, 6, 7, 8, 9, 10, 20, 30, 40, 50, 60, 80, or 100 μM when the chemotherapy drug is tested on the cancer cell line(s). In some embodiments, the predetermined nonresponsive score is indicative of a IC₅₀ of greater than 1 μM when the chemotherapy drug is tested on the cancer cell line(s). In some embodiments, the predetermined nonresponsive score is indicative of a IC₅₀ of greater than about 0.5, 1, 2, 3, 4, 5, 6, 7, 8, 9, 10, 20, 30, 40, 50, 60, 80, or 100 μM when the chemotherapy drug is tested on the cancer cell line(s). In some embodiments, the predetermined responsive score is 0, and the predetermined nonresponsive score is 1. In some embodiments, classifying the subject comprises classifying the subject as responsive when the expression score is lower than 0.4. In some embodiments, classifying the subject comprises classifying the subject as non-responsive when the expression score is greater than 0.6.

In some embodiments, a subject is responsive to a chemotherapy if the rate of cancer/tumor growth is inhibited as a result of contact with the chemotherapy agent, compared to its growth in the absence of contact with the chemotherapy agent. Growth of a cancer can be measured in a variety of ways. For instance, the size of a tumor or measuring the expression of tumor markers appropriate for that tumor type.

In some embodiments, a subject is non-responsive to a chemotherapy if its rate of cancer/tumor growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or measuring the expression of tumor markers appropriate for that tumor type. Measures of non-responsiveness can be assessed using additional criteria beyond growth size of a tumor such as, but not limited to, patient quality of life, and degree of metastases.

The method described herein can include a step of determining an expression score. The expression score can be determined by using the expression levels of certain biomarkers in a subject sample set.

The method described herein can include a step of determining the expression profiles. In certain embodiments, the expression profile obtained is a genomic or nucleic acid expression profile, where the amount or level of one or more nucleic acids in the sample is determined. In these embodiments, the sample that is assayed to generate the expression profile employed in the diagnostic or prognostic methods is one that is a nucleic acid sample. The nucleic acid sample includes a population of nucleic acids that includes the expression information of the phenotype determinative biomarkers of the cell or tissue being analyzed. In some embodiments, the nucleic acid may include mRNA. In some embodiments, the nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as isolated, amplified, or employed to prepare cDNA, cRNA, etc., as is known in the field of differential gene expression. Accordingly, determining the level of mRNA in a sample includes preparing cDNA or cRNA from the mRNA and subsequently measuring the cDNA or cRNA. The sample is typically prepared from a cell or tissue harvested from a subject in need of treatment, e.g., via biopsy of tissue, using standard protocols, where cell types or tissues from which such nucleic acids may be generated include any tissue in which the expression pattern of the to be determined phenotype exists, including, but not limited to, disease cells or tissue, body fluids, etc.

The expression level may be generated from the initial nucleic acid sample using any convenient protocol. While a variety of different manners of generating expression levels are known, such as those employed in the field of differential gene expression/biomarker analysis, one representative and convenient type of protocol for generating expression levels is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In some embodiments, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

The method described herein includes a step of taking a subject sample. In certain exemplary embodiments, the subject sample comprises cancer tissue samples, such as archived samples. The subject sample set is preferably derived from cancer tissue samples having been characterized by prognosis, likelihood of recurrence, long term survival, clinical outcome, treatment response, diagnosis, cancer classification, or personalized genomics profile. The sample can be blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, nipple aspirate, bronchial aspirate, synovial fluid, joint aspirate, ascites, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid samples. The sample can include materials containing homogenized solid material, such as from a stool sample, a tissue sample, or a tissue biopsy, for example. The sample can also include materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Samples can also be collected, e.g., by micro dissection (e.g., laser capture micro dissection (LCM) or laser micro dissection (LMD)), bladder wash, smear (e.g., a PAP smear), or ductal lavage. A sample obtained or derived from an individual includes any such sample that has been processed in any suitable manner after being obtained from the individual, for example, fresh frozen or formalin fixed and/or paraffin embedded.

The methods described herein includes administering one or more tubulin binding agents to the selected subject. In some embodiments, the tubulin binding agent is plinabulin. In some embodiments, the tubulin binding agent is colchicine.

In some embodiments, the tubulin binding agent (e.g., plinabulin) is administered at a dose in the range of about 1-50 mg/m² of the body surface area. In some embodiments, the tubulin binding agent (e.g., plinabulin) is administered at a dose in the range of about 5 to about 50 mg/m² of the body surface area. In some embodiments, the tubulin binding agent (e.g., plinabulin) is administered at a dose in the range of about 20 to about 40 mg/m² of the body surface area. In some embodiments, the tubulin binding agent (e.g., plinabulin) is administered at a dose in the range of about 15 to about 30 mg/m² of the body surface area. In some embodiments, the tubulin binding agent (e.g., plinabulin) is administered at a dose in the range of about 0.5-1, 0.5-2, 0.5-3, 0.5-4, 0.5-5, 0.5-6, 0.5-7, 0.5-8, 0.5-9, 0.5-10, 0.5-11, 0.5-12, 0.5-13, 0.5-13.75, 0.5-14, 0.5-15, 0.5-16, 0.5-17, 0.5-18, 0.5-19, 0.5-20, 0.5-22.5, 0.5-25, 0.5-27.5, 0.5-30, 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, t 2.5-22.5, or 9.5-21.5 mg/m², of the body surface area. In some embodiments, the tubulin binding agent (e.g., plinabulin) is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the tubulin binding agent (e.g., plinabulin) is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, or 40 mg/m² of the body surface area. In some embodiments, the tubulin binding agent (e.g., plinabulin) is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, or 50 mg/m² of the body surface area. In some embodiments, the tubulin binding agent (e.g., plinabulin) is administered at a dose of about 10, 13.5, 20, or 30 mg/m² of the body surface area. In some embodiments, the tubulin binding agent (e.g., plinabulin) is administered at a dose of about 20 mg/m² of the body surface area.

In some embodiments, the tubulin binding agent (e.g., plinabulin) dose is about 5 mg-100 mg, or about 10 mg-80 mg. In some embodiments, the tubulin binding agent (e.g., plinabulin) dose is about 15 mg-100 mg, or about 20 mg-80 mg. In some embodiments, the tubulin binding agent (e.g., plinabulin) is administered at a dose in the range of about 15 mg-60 mg. In some embodiments, the tubulin binding agent (e.g., plinabulin) dose is about 0.5 mg-3 mg, 0.5 mg-2 mg, 0.75 mg-2 mg, 1 mg-10 mg, 1.5 mg-10 mg, 2 mg-10 mg, 3 mg-10 mg, 4 mg-10 mg, 1 mg-8 mg, 1.5 mg-8 mg, 2 mg-8 mg, 3 mg-8 mg, 4 mg-8 mg, 1 mg-6 mg, 1.5 mg-6 mg, 2 mg-6 mg, 3 mg-6 mg, or about 4 mg-6 mg. In some embodiments, the tubulin binding agent (e.g., plinabulin) is administered at about 2 mg-6 mg or 2 mg-4.5 mg. In some embodiments, the tubulin binding agent (e.g., plinabulin) is administered at about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the tubulin binding agent (e.g., plinabulin) dose is greater than about 0.5 mg, 1 mg, 1.5 mg, 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, or about 40 mg. In some embodiments, the tubulin binding agent (e.g., plinabulin) dose is about less than about 1 mg, 1.5 mg, 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, about 40 mg, or about 50 mg.

In some embodiments, the cancer can include leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, and the like. As used herein, colorectal cancer encompasses cancers that may involve cancer in tissues of both the rectum and other portions of the colon as well as cancers that may be individually classified as either colon cancer or rectal cancer. In one embodiment, the methods described herein refer to cancers that are treated with anti-angiogenic agents, anti-angiogenic targeted therapies, inhibitors of angiogenesis signaling, but not limited to these classes. These cancers also include subclasses and subtypes of these cancers at various stages of pathogenesis. In certain exemplary embodiments, the cancer is central nervous system (CNS) lymphoma, lung cancer, breast cancer, ovarian cancer, and prostate cancer. In some embodiments, the cancer is a non-small cell lung cancer.

In some embodiments, the biomarker described herein can be an mRNA associated with an expression level of the genes described herein, and also any and all probesets that reflect the expression of genes that can be used to predict a patient's response to a tubulin binding agent, and the probesets with or without gene annotation that have been identified as predictive of a tubulin binding agent's activity and/or differentially expressed in a tubulin binding agent's active versus inactive cell lines.

The biomarkers described herein can be an mRNA associated with one or more probesets suitable for detecting the gene expression in at least one cancer cell line. In some embodiments, the biomarker described herein can be one or more mRNA associated with the probesets listed in Table 1, Table 2, or Table 4. In some embodiments, the biomarker described herein can be one or more mRNA identifiable using the probesets listed in Table 1, Table 2, or Table 4.

TABLE 1
Probeset selected from binary logistic
regression versus Plinabulin Activity

| Probeset (x value) | Count | PValue |
|---|---|---|
| 205525_at | 43 | 4.95E−08 |
| 201617_x_at | 43 | 5.84E−07 |
| 235834_at | 43 | 1.41E−06 |
| 241627_x_at | 43 | 1.77E−06 |
| 228647_at | 43 | 2.22E−06 |
| 212077_at | 43 | 2.66E−06 |
| 225504_at | 43 | 3.47E−06 |
| 213125_at | 43 | 4.27E−06 |
| 224753_at | 43 | 5.91E−06 |
| 215983_s_at | 43 | 6.52E−06 |
| 236165_at | 43 | 6.77E−06 |
| 201616_s_at | 43 | 0.000011051 |
| 205998_x_at | 43 | 1.25997E−05 |
| 200894_s_at | 43 | 0.000013931 |
| 217667_at | 43 | 1.47694E−05 |
| 240038_at | 43 | 0.000017112 |
| 233019_at | 43 | 2.02595E−05 |
| 200895_s_at | 43 | 2.12115E−05 |
| 204837_at | 43 | 3.03053E−05 |
| 215398_at | 43 | 3.54566E−05 |
| 212239_at | 43 | 3.57246E−05 |
| 209448_at | 43 | 3.81034E−05 |
| 232522_at | 43 | 4.10017E−05 |
| 218836_at | 43 | 4.19118E−05 |
| 242808_at | 43 | 5.07722E−05 |
| 202450_s_at | 43 | 5.29235E−05 |
| 226848_at | 43 | 5.31599E−05 |
| 221729_at | 43 | 5.41473E−05 |
| 212450_at | 43 | 5.43849E−05 |
| 233626_at | 43 | 5.93985E−05 |
| 221616_s_at | 43 | 5.97619E−05 |
| 221730_at | 43 | 0.000060174 |
| 201342_at | 43 | 6.74735E−05 |
| 201615_x_at | 43 | 6.92249E−05 |
| 223641_at | 43 | 7.18308E−05 |
| 202594_at | 43 | 7.18956E−05 |
| 232372_at | 43 | 7.42411E−05 |
| 230118_at | 43 | 0.000079248 |
| 239238_at | 43 | 7.97361E−05 |
| 201907_x_at | 43 | 8.71338E−05 |
| 219648_at | 43 | 8.82503E−05 |
| 224479_s_at | 43 | 8.87103E−05 |
| 201312_s_at | 43 | 9.14151E−05 |
| 1562434_at | 43 | 9.30091E−05 |
| 238119_at | 43 | 0.000099813 |
| 233263_at | 43 | 0.000101024 |
| 229773_at | 43 | 0.000103434 |
| 230370_x_at | 43 | 0.000108975 |
| 1558501_at | 43 | 0.000110185 |
| 215418_at | 43 | 0.000112808 |
| 236154_at | 43 | 0.000113915 |
| 1562948_at | 43 | 0.000116232 |
| 219429_at | 43 | 0.000116255 |
| 235756_at | 43 | 0.000135807 |
| 200809_x_at | 43 | 0.000136005 |
| 224417_at | 43 | 0.00014047 |
| 240008_at | 43 | 0.000140473 |
| 209549_s_at | 43 | 0.000143614 |
| 213227_at | 43 | 0.000147285 |
| 236703_at | 43 | 0.000148232 |
| 226661_at | 43 | 0.000148506 |
| 219786_at | 43 | 0.000159996 |
| 212700_x_at | 43 | 0.000163484 |
| 213695_at | 43 | 0.000168208 |
| 232175_at | 43 | 0.000168506 |
| 213278_at | 43 | 0.000170228 |
| 218321_x_at | 43 | 0.000170598 |
| 212778_at | 43 | 0.000177408 |
| 210235_s_at | 43 | 0.000178814 |
| 226785_at | 43 | 0.000180774 |
| 1559600_at | 43 | 0.000182243 |
| 200658_s_at | 43 | 0.000185447 |
| 207180_s_at | 43 | 0.000188671 |
| 218978_s_at | 43 | 0.00018966 |
| 235796_at | 43 | 0.000189753 |
| 203867_s_at | 43 | 0.000193271 |
| 221543_s_at | 43 | 0.000194046 |
| 221542_s_at | 43 | 0.000195428 |
| 209889_at | 43 | 0.000196073 |
| 218567_x_at | 43 | 0.000197353 |
| 227685_at | 43 | 0.000199343 |
| 232459_at | 43 | 0.000201142 |
| 202811_at | 43 | 0.000201185 |
| 239999_at | 43 | 0.000201341 |
| 244674_at | 43 | 0.000201555 |
| 201483_s_at | 43 | 0.000203092 |
| 213077_at | 43 | 0.000215051 |
| 220525_s_at | 43 | 0.000230829 |
| 200022_at | 43 | 0.000234811 |
| 233674_at | 43 | 0.000239101 |
| 241906_at | 43 | 0.000240472 |
| 212301_at | 43 | 0.000243373 |
| 205381_at | 43 | 0.000245075 |
| 235114_x_at | 43 | 0.000246938 |
| 204076_at | 43 | 0.000248178 |
| 208109_s_at | 43 | 0.000248921 |
| 243088_at | 43 | 0.000252876 |
| 231106_at | 43 | 0.000255388 |
| 239519_at | 43 | 0.000258932 |
| 224359_s_at | 43 | 0.000260533 |
| 208009_s_at | 43 | 0.000268888 |
| 205428_s_at | 43 | 0.000271614 |
| 219050_s_at | 43 | 0.000274312 |
| 224755_at | 43 | 0.00027563 |
| 232353_s_at | 43 | 0.000276335 |
| 202518_at | 43 | 0.00028837 |
| 1570338_at | 43 | 0.000299695 |
| 205103_at | 43 | 0.00030208 |
| 214862_x_at | 43 | 0.000303773 |
| 214937_x_at | 43 | 0.000304058 |
| 243361_at | 43 | 0.000311375 |
| 236192_at | 43 | 0.000316771 |
| 225217_s_at | 43 | 0.00032131 |
| 221814_at | 43 | 0.000321518 |
| 219350_s_at | 43 | 0.000321738 |
| 232682_at | 43 | 0.000323259 |
| 227262_at | 43 | 0.000324127 |
| 224467_s_at | 43 | 0.000325338 |
| 205613_at | 43 | 0.000327504 |
| 1554063_at | 43 | 0.0003286 |
| 200847_s_at | 43 | 0.000335082 |
| 243084_at | 43 | 0.000336737 |
| 1557238_s_at | 43 | 0.000337016 |
| 233982_x_at | 43 | 0.000354379 |
| 203816_at | 43 | 0.000360012 |
| 212116_at | 43 | 0.000364228 |
| 211813_x_at | 43 | 0.000367346 |
| 219469_at | 43 | 0.000371421 |
| 211161_s_at | 43 | 0.000371692 |
| 227102_at | 43 | 0.000387104 |
| 225728_at | 43 | 0.00038894 |
| 221998_s_at | 43 | 0.000389886 |
| 1553275_s_at | 43 | 0.000395103 |
| 209911_x_at | 43 | 0.000398959 |
| 1559776_at | 43 | 0.000400911 |
| 236531_at | 43 | 0.000401616 |
| 229215_at | 43 | 0.000402093 |
| 230487_at | 43 | 0.000404246 |
| 201307_at | 43 | 0.000410269 |
| 231881_at | 43 | 0.000411268 |
| 41037_at | 43 | 0.000411462 |
| 235786_at | 43 | 0.000413747 |
| 203612_at | 43 | 0.000418135 |
| 238146_at | 43 | 0.000419695 |
| 205704_s_at | 43 | 0.000422399 |
| 225460_at | 43 | 0.000426673 |
| 1559332_at | 43 | 0.000427136 |
| 229022_at | 43 | 0.000430345 |
| 213070_at | 43 | 0.000431257 |
| 201763_s_at | 43 | 0.000432728 |
| 238299_at | 43 | 0.000432857 |
| 201893_x_at | 43 | 0.000440727 |
| 213308_at | 43 | 0.000448735 |
| 230071_at | 43 | 0.000449363 |
| 201311_s_at | 43 | 0.000450514 |
| 227221_at | 43 | 0.000453591 |
| 235071_at | 43 | 0.000462091 |
| 233759_s_at | 43 | 0.00046475 |
| 233678_at | 43 | 0.000468274 |
| 230035_at | 43 | 0.000475374 |
| 209165_at | 43 | 0.000479462 |
| 203131_at | 43 | 0.000482403 |
| 227693_at | 43 | 0.000483737 |
| 229073_at | 43 | 0.000484769 |
| 211725_s_at | 43 | 0.000484952 |
| 224892_at | 43 | 0.000487632 |
| 218147_s_at | 43 | 0.000497781 |
| 203791_at | 43 | 0.000500345 |
| 218405_at | 43 | 0.000504383 |
| 222551_s_at | 43 | 0.000505299 |
| 217781_s_at | 43 | 0.000505952 |
| 226007_at | 43 | 0.00050657 |
| 1558401_at | 43 | 0.000506807 |
| 228185_at | 43 | 0.00050737 |
| 202174_s_at | 43 | 0.000513713 |
| 213307_at | 43 | 0.000515086 |
| 201852_x_at | 43 | 0.000515809 |
| 232527_at | 43 | 0.000525921 |
| 219906_at | 43 | 0.00052664 |
| 232057_at | 43 | 0.000529648 |
| 208907_s_at | 43 | 0.000534009 |
| 230702_at | 43 | 0.000536635 |
| 210236_at | 43 | 0.000538265 |
| 241970_at | 43 | 0.000539955 |
| 228189_at | 43 | 0.000543155 |
| 212110_at | 43 | 0.000544524 |
| 228603_at | 43 | 0.000549906 |
| 227687_at | 43 | 0.00055117 |
| 229665_at | 43 | 0.00056582 |
| 225725_at | 43 | 0.00056746 |
| 235817_at | 43 | 0.000568807 |
| 216995_x_at | 43 | 0.000573488 |
| 1552330_at | 43 | 0.00057382 |
| 204828_at | 43 | 0.000573827 |
| 215433_at | 43 | 0.00057477 |
| 222470_s_at | 43 | 0.000584144 |
| 206074_s_at | 43 | 0.000590901 |
| 230905_at | 43 | 0.00059309 |
| 214118_x_at | 43 | 0.000599662 |
| 239476_at | 43 | 0.000602281 |
| 226241_s_at | 43 | 0.000603342 |
| 236496_at | 43 | 0.000603781 |
| 229205_at | 43 | 0.000606524 |
| 224935_at | 43 | 0.000606625 |
| 244026_at | 43 | 0.000608107 |
| 243591_at | 43 | 0.000608542 |
| 207493_x_at | 43 | 0.000609116 |
| 1565752_at | 43 | 0.000626101 |
| 201045_s_at | 43 | 0.000629249 |
| 236375_at | 43 | 0.000658993 |
| 1555751_a_at | 43 | 0.000659405 |
| 1558802_at | 43 | 0.00066172 |
| 240991_at | 43 | 0.000676941 |
| 204808_s_at | 43 | 0.000677033 |
| 235198_at | 43 | 0.000681041 |
| 201533_at | 43 | 0.000681466 |
| 229289_at | 43 | 0.000685572 |
| 221606_s_at | 43 | 0.00068756 |
| 243259_at | 43 | 0.000691684 |
| 219526_at | 43 | 0.00070142 |
| 228359_at | 43 | 0.000706475 |
| 230606_at | 43 | 0.000710642 |
| 214083_at | 43 | 0.000712743 |
| 242549_at | 43 | 0.000712859 |
| 229287_at | 43 | 0.000713439 |
| 243253_at | 43 | 0.000719817 |
| 209219_at | 43 | 0.000720265 |
| 207143_at | 43 | 0.000721495 |
| 212172_at | 43 | 0.000726953 |
| 202339_at | 43 | 0.000737394 |
| 235588_at | 43 | 0.000740693 |
| 202029_x_at | 43 | 0.000743277 |
| 228170_at | 43 | 0.000745217 |
| 213155_at | 43 | 0.000746114 |
| 211542_x_at | 43 | 0.000746149 |
| 235031_at | 43 | 0.000746268 |
| 1563467_at | 43 | 0.000754386 |
| 235318_at | 43 | 0.000755183 |
| 241938_at | 43 | 0.000763046 |
| 1568815_a_at | 43 | 0.000765287 |
| 226416_at | 43 | 0.000767385 |
| 200936_at | 43 | 0.00077086 |
| 226502_at | 43 | 0.00077135 |
| 209656_s_at | 43 | 0.00077146 |
| 214814_at | 43 | 0.000776266 |
| 223060_at | 43 | 0.000777989 |
| 241956_at | 43 | 0.000779608 |
| 202831_at | 43 | 0.000788951 |
| 202564_x_at | 43 | 0.000791639 |
| 202380_s_at | 43 | 0.000798559 |
| 225885_at | 43 | 0.000798765 |
| 243681_at | 43 | 0.000800331 |
| 227806_at | 43 | 0.000808613 |
| 215545_at | 43 | 0.000810434 |
| 232148_at | 43 | 0.000811815 |
| 222344_at | 43 | 0.000812565 |
| 228407_at | 43 | 0.000813711 |
| 224619_at | 43 | 0.000820646 |
| 243801_x_at | 43 | 0.000823204 |
| 200088_x_at | 43 | 0.000823394 |
| 211896_s_at | 43 | 0.00083362 |
| 212440_at | 43 | 0.000835657 |
| 218577_at | 43 | 0.000837225 |
| 219455_at | 43 | 0.000837307 |
| 219148_at | 43 | 0.000844765 |
| 1558275_at | 43 | 0.000847598 |
| 200095_x_at | 43 | 0.000848631 |
| 204281_at | 43 | 0.000849142 |
| 240868_at | 43 | 0.000855914 |
| 1560926_at | 43 | 0.000857583 |
| 242007_at | 43 | 0.000860842 |
| 32541_at | 43 | 0.000864416 |
| 201346_at | 43 | 0.000865436 |
| 228132_at | 43 | 0.000868232 |
| 1561135_at | 43 | 0.000871157 |
| 223305_at | 43 | 0.000875369 |
| 232882_at | 43 | 0.00089771 |
| 240815_at | 43 | 0.000897967 |
| 229398_at | 43 | 0.000908042 |
| 219071_x_at | 43 | 0.000912068 |
| 211698_at | 43 | 0.000912446 |
| 244693_at | 43 | 0.000917772 |
| 32402_s_at | 43 | 0.000917874 |
| 228338_at | 43 | 0.000924358 |
| 242701_at | 43 | 0.000930169 |
| 225144_at | 43 | 0.000934694 |
| 207605_x_at | 43 | 0.000935283 |
| 239867_at | 43 | 0.000937459 |
| 218762_at | 43 | 0.000940641 |
| 225223_at | 43 | 0.000945816 |
| 1560579_s_at | 43 | 0.000947662 |
| 232510_s_at | 43 | 0.000948144 |
| 222460_s_at | 43 | 0.000949853 |
| 1554411_at | 43 | 0.000961591 |
| 220094_s_at | 43 | 0.000966318 |
| 244414_at | 43 | 0.000966678 |
| 212202_s_at | 43 | 0.000971767 |
| 201268_at | 43 | 0.000974235 |
| 224876_at | 43 | 0.000993706 |
| 226488_at | 43 | 0.001001139 |
| 1556818_at | 43 | 0.001005662 |
| 239258_at | 43 | 0.001014051 |
| 213102_at | 43 | 0.001014741 |
| 225878_at | 43 | 0.00101585 |
| 233588_x_at | 43 | 0.001017813 |
| 235646_at | 43 | 0.001017834 |
| 203606_at | 43 | 0.001023472 |
| 218125_s_at | 43 | 0.001024317 |
| 230394_at | 43 | 0.001026373 |
| 228587_at | 43 | 0.001034942 |
| 226395_at | 43 | 0.001057428 |
| 238002_at | 43 | 0.001059292 |
| 242558_at | 43 | 0.00106078 |
| 219479_at | 43 | 0.00107176 |
| 213322_at | 43 | 0.001071826 |
| 1553157_at | 43 | 0.001076854 |
| 215980_s_at | 43 | 0.001077386 |
| 223406_x_at | 43 | 0.001081032 |
| 1557478_at | 43 | 0.001081844 |
| 1556126_s_at | 43 | 0.001083572 |
| 224248_x_at | 43 | 0.001084219 |
| 226742_at | 43 | 0.001087831 |
| 225837_at | 43 | 0.001087988 |
| 214132_at | 43 | 0.001088429 |
| 240793_at | 43 | 0.001089248 |
| 200877_at | 43 | 0.001091003 |
| 214394_x_at | 43 | 0.00109474 |
| 242208_at | 43 | 0.001095758 |
| 241965_at | 43 | 0.001100179 |
| 218050_at | 43 | 0.001111383 |
| 221251_x_at | 43 | 0.001121289 |
| 239571_at | 43 | 0.001122377 |
| 1559023_a_at | 43 | 0.001123266 |
| 221505_at | 43 | 0.001127063 |
| 229053_at | 43 | 0.001133438 |
| 206506_s_at | 43 | 0.001139389 |
| 1558216_at | 43 | 0.001140572 |
| 200931_s_at | 43 | 0.001142367 |
| 213099_at | 43 | 0.001145678 |
| 229483_at | 43 | 0.001145929 |
| 224704_at | 43 | 0.001151887 |
| 1558458_at | 43 | 0.001161121 |
| 217975_at | 43 | 0.00116337 |
| 213189_at | 43 | 0.001164331 |
| 202080_s_at | 43 | 0.001166869 |
| 202494_at | 43 | 0.001175244 |
| 231986_at | 43 | 0.00117894 |
| 244659_at | 43 | 0.00119689 |
| 222096_x_at | 43 | 0.001202607 |
| 233274_at | 43 | 0.001210757 |
| 222267_at | 43 | 0.00121679 |
| 237561_x_at | 43 | 0.00121877 |
| 243158_at | 43 | 0.001219691 |
| 201393_s_at | 43 | 0.001220989 |
| 235792_x_at | 43 | 0.001227759 |
| 214948_s_at | 43 | 0.001229536 |
| 1556820_a_at | 43 | 0.001231565 |
| 221081_s_at | 43 | 0.001240779 |
| 226713_at | 43 | 0.001242447 |
| 213703_at | 43 | 0.001257878 |
| 242576_x_at | 43 | 0.001263721 |
| 232081_at | 43 | 0.001263735 |
| 1554474_a_at | 43 | 0.001265234 |
| 219662_at | 43 | 0.001273785 |
| 228871_at | 43 | 0.001276192 |
| 201684_s_at | 43 | 0.001283257 |
| 201484_at | 43 | 0.001288369 |
| 213145_at | 43 | 0.001288577 |
| 217910_x_at | 43 | 0.001288981 |
| 218136_s_at | 43 | 0.001291531 |
| 203420_at | 43 | 0.001294244 |
| 225743_at | 43 | 0.001296887 |
| 233648_at | 43 | 0.001300519 |
| 214880_x_at | 43 | 0.001301354 |
| 225318_at | 43 | 0.001309075 |
| 213157_s_at | 43 | 0.001313773 |
| 217555_at | 43 | 0.00132501 |
| 200666_s_at | 43 | 0.001325389 |
| 1557737_s_at | 43 | 0.001329159 |
| 37892_at | 43 | 0.001331655 |
| 242092_at | 43 | 0.001334125 |
| 226314_at | 43 | 0.001335987 |
| 218823_s_at | 43 | 0.001346304 |
| 242677_at | 43 | 0.001355834 |
| 223728_at | 43 | 0.001356201 |
| 225384_at | 43 | 0.001358413 |
| 216439_at | 43 | 0.001358617 |
| 216115_at | 43 | 0.001359161 |
| 238871_at | 43 | 0.001359369 |
| 227103_s_at | 43 | 0.001371602 |
| 224565_at | 43 | 0.001371995 |
| 205333_s_at | 43 | 0.001372076 |
| 34406_at | 43 | 0.00137388 |
| 203927_at | 43 | 0.00137517 |
| 210848_at | 43 | 0.001376373 |
| 214372_x_at | 43 | 0.001384608 |
| 238735_at | 43 | 0.001386645 |
| 234813_at | 43 | 0.001389291 |
| 217935_s_at | 43 | 0.001390121 |
| 238524_at | 43 | 0.00139111 |
| 222821_s_at | 43 | 0.00139299 |
| 225948_at | 43 | 0.00139495 |
| 235632_at | 43 | 0.001395066 |
| 241897_at | 43 | 0.001395317 |
| 226565_at | 43 | 0.001396402 |
| 230656_s_at | 43 | 0.001402733 |
| 203955_at | 43 | 0.001403264 |
| 219241_x_at | 43 | 0.001403362 |
| 234661_at | 43 | 0.001416171 |
| 227042_at | 43 | 0.001425707 |
| 232865_at | 43 | 0.001426829 |
| 236435_at | 43 | 0.001427436 |
| 228693_at | 43 | 0.001427475 |
| 240482_at | 43 | 0.001446105 |
| 236367_at | 43 | 0.001446499 |
| 216005_at | 43 | 0.00145499 |
| 224875_at | 43 | 0.001461015 |
| 219594_at | 43 | 0.001464958 |
| 214678_x_at | 43 | 0.00146536 |
| 1556180_at | 43 | 0.00147102 |
| 222111_at | 43 | 0.001475234 |
| 90265_at | 43 | 0.001481798 |
| 205906_at | 43 | 0.001488218 |
| 233101_at | 43 | 0.001488435 |
| 217610_at | 43 | 0.001489801 |
| 225074_at | 43 | 0.001492524 |
| 213905_x_at | 43 | 0.001500613 |
| 210946_at | 43 | 0.001500649 |
| 244610_x_at | 43 | 0.001501295 |
| 219531_at | 43 | 0.001505693 |
| 237065_s_at | 43 | 0.001506035 |
| 212351_at | 43 | 0.001518862 |
| 202379_s_at | 43 | 0.001519523 |
| 237131_at | 43 | 0.001521622 |
| 213414_s_at | 43 | 0.001523112 |
| 1557242_at | 43 | 0.001554821 |
| 233309_at | 43 | 0.001555986 |
| 239557_at | 43 | 0.00156159 |
| 1557586_s_at | 43 | 0.001573083 |
| 211992_at | 43 | 0.001576721 |
| 212369_at | 43 | 0.001581504 |
| 1555878_at | 43 | 0.001592589 |
| 210449_x_at | 43 | 0.00159504 |
| 239809_at | 43 | 0.001597807 |
| 226317_at | 43 | 0.001605413 |
| 214731_at | 43 | 0.00160761 |
| 228646_at | 43 | 0.001616958 |
| 216524_x_at | 43 | 0.001624191 |
| 224903_at | 43 | 0.00162874 |
| 217786_at | 43 | 0.001633736 |
| 242514_at | 43 | 0.001634916 |
| 232371_at | 43 | 0.001639987 |
| 1556657_at | 43 | 0.00164904 |
| 222310_at | 43 | 0.00165349 |
| 236623_at | 43 | 0.00166003 |
| 242053_at | 43 | 0.001662841 |
| 200817_x_at | 43 | 0.00168027 |
| 239253_at | 43 | 0.001680847 |
| 237208_at | 43 | 0.001687662 |
| 1558329_at | 43 | 0.001690699 |
| 223048_at | 43 | 0.001697303 |
| 1554213_at | 43 | 0.00170363 |
| 1564906_at | 43 | 0.001712385 |
| 227033_at | 43 | 0.001724024 |
| 233480_at | 43 | 0.001724711 |
| 235220_at | 43 | 0.001732526 |
| 230177_at | 43 | 0.001737999 |
| 227628_at | 43 | 0.001739324 |
| 222304_x_at | 43 | 0.001741195 |
| 1556178_x_at | 43 | 0.001743052 |
| 201901_s_at | 43 | 0.001750685 |
| 228803_at | 43 | 0.001753313 |
| 236080_at | 43 | 0.001763049 |
| 241993_x_at | 43 | 0.001764344 |
| 209428_s_at | 43 | 0.00177168 |
| 236201_at | 43 | 0.001775124 |
| 1569999_at | 43 | 0.001781989 |
| 243673_at | 43 | 0.00179823 |
| 209341_s_at | 43 | 0.001802244 |
| 1558466_at | 43 | 0.00180478 |
| 200686_s_at | 43 | 0.001806777 |
| 231437_at | 43 | 0.001810425 |
| 241888_at | 43 | 0.001811277 |
| 207829_s_at | 43 | 0.001812362 |
| 235396_at | 43 | 0.001819464 |
| 225696_at | 43 | 0.001822744 |
| 220093_at | 43 | 0.001823964 |
| 200036_s_at | 43 | 0.001826676 |
| 207000_s_at | 43 | 0.001828328 |
| 205991_s_at | 43 | 0.001830412 |
| 204403_x_at | 43 | 0.001836271 |
| 238550_at | 43 | 0.001841909 |
| 215209_at | 43 | 0.001843522 |
| 205200_at | 43 | 0.001844369 |
| 235576_at | 43 | 0.001847865 |
| 221712_s_at | 43 | 0.00184854 |
| 1569149_at | 43 | 0.001848581 |
| 201701_s_at | 43 | 0.001848772 |
| 211064_at | 43 | 0.001850041 |
| 211806_s_at | 43 | 0.001857524 |
| 234074_at | 43 | 0.001859785 |
| 235359_at | 43 | 0.001863173 |
| 215076_s_at | 43 | 0.001864247 |
| 239207_at | 43 | 0.001864863 |
| 242696_at | 43 | 0.001872167 |
| 222029_x_at | 43 | 0.001893208 |
| 1570078_a_at | 43 | 0.001894469 |
| 1569150_x_at | 43 | 0.001902173 |
| 229789_at | 43 | 0.001920508 |
| 218732_at | 43 | 0.001922152 |
| 232478_at | 43 | 0.001928149 |
| 229218_at | 43 | 0.001933592 |
| 212946_at | 43 | 0.001934371 |
| 242971_at | 43 | 0.001934643 |
| 214967_at | 43 | 0.001937473 |
| 244433_at | 43 | 0.001944736 |
| 216883_x_at | 43 | 0.001949712 |
| 236649_at | 43 | 0.001969959 |
| 232521_at | 43 | 0.001973671 |
| 240261_at | 43 | 0.001979355 |
| 238342_at | 43 | 0.001991157 |
| 203644_s_at | 43 | 0.001994904 |
| 243046_at | 43 | 0.001997366 |
| 233114_at | 43 | 0.001999039 |
| 232613_at | 43 | 0.002002042 |
| 218885_s_at | 43 | 0.002006843 |
| 218803_at | 43 | 0.002023351 |
| 1556338_at | 43 | 0.002031646 |
| 241808_at | 43 | 0.002039612 |
| 229031_at | 43 | 0.002041969 |
| 217713_x_at | 43 | 0.002042686 |
| 242688_at | 43 | 0.002044868 |
| 223056_s_at | 43 | 0.002047987 |
| 1565804_at | 43 | 0.002054688 |
| 222475_at | 43 | 0.002059344 |
| 243981_at | 43 | 0.002060812 |
| 212465_at | 43 | 0.002061135 |
| 203095_at | 43 | 0.002072614 |
| 236454_at | 43 | 0.002073197 |
| 212630_at | 43 | 0.002073856 |
| 203903_s_at | 43 | 0.002077158 |
| 208600_s_at | 43 | 0.002082792 |
| 242968_at | 43 | 0.002082864 |
| 238728_at | 43 | 0.0020862 |
| 213089_at | 43 | 0.002089829 |
| 222186_at | 43 | 0.002093465 |
| 233770_at | 43 | 0.002107756 |
| 200812_at | 43 | 0.002125819 |
| 232704_s_at | 43 | 0.002130369 |
| 201584_s_at | 43 | 0.002149329 |
| 223619_x_at | 43 | 0.002151924 |
| 207808_s_at | 43 | 0.002158761 |
| 225086_at | 43 | 0.002159432 |
| 233607_at | 43 | 0.002159529 |
| 213909_at | 43 | 0.00216747 |
| 1557543_at | 43 | 0.002174055 |
| 239952_at | 43 | 0.002182457 |
| 218316_at | 43 | 0.00218344 |
| 224743_at | 43 | 0.002186577 |
| 239721_at | 43 | 0.002192154 |
| 210553_x_at | 43 | 0.002197665 |
| 213621_s_at | 43 | 0.002198289 |
| 230443_at | 43 | 0.002203609 |
| 212626_x_at | 43 | 0.002204329 |
| 204575_s_at | 43 | 0.002218698 |
| 214271_x_at | 43 | 0.002224312 |
| 221834_at | 43 | 0.002237627 |
| 230305_at | 43 | 0.002239548 |
| 236858_s_at | 43 | 0.002243288 |
| 202649_x_at | 43 | 0.002250174 |
| 226873_at | 43 | 0.002251576 |
| 213174_at | 43 | 0.002257183 |
| 207223_s_at | 43 | 0.002258257 |
| 201934_at | 43 | 0.002259813 |
| 231852_at | 43 | 0.002263956 |
| 242871_at | 43 | 0.002282325 |
| 232568_at | 43 | 0.002288321 |
| 216147_at | 43 | 0.002298611 |
| 236764_at | 43 | 0.002301324 |
| 212229_s_at | 43 | 0.002301811 |
| 228946_at | 43 | 0.002313254 |
| 231146_at | 43 | 0.002320062 |
| 227075_at | 43 | 0.002321211 |
| 229541_at | 43 | 0.002323126 |
| 240964_at | 43 | 0.002323888 |
| 205146_x_at | 43 | 0.002324356 |
| 211488_s_at | 43 | 0.002328452 |
| 218155_x_at | 43 | 0.002338674 |
| 242283_at | 43 | 0.002339652 |
| 230991_at | 43 | 0.002339681 |
| 224865_at | 43 | 0.002340533 |
| 212721_at | 43 | 0.002348464 |
| 232641_at | 43 | 0.002351441 |
| 228396_at | 43 | 0.00235528 |
| 209655_s_at | 43 | 0.002355672 |
| 210242_x_at | 43 | 0.002356604 |
| 242669_at | 43 | 0.002358119 |
| 212887_at | 43 | 0.002359938 |
| 226179_at | 43 | 0.002372628 |
| 203911_at | 43 | 0.002385507 |
| 236855_at | 43 | 0.00238835 |
| 210213_s_at | 43 | 0.002390702 |
| 1558142_at | 43 | 0.002403381 |
| 205759_s_at | 43 | 0.0024092 |
| 219628_at | 43 | 0.002413743 |
| 239672_at | 43 | 0.00242623 |
| 233599_at | 43 | 0.002426413 |
| 235437_at | 43 | 0.002428148 |
| 234734_s_at | 43 | 0.002428897 |
| 218481_at | 43 | 0.002429571 |
| 203004_s_at | 43 | 0.002431501 |
| 231727_s_at | 43 | 0.002446748 |
| 220355_s_at | 43 | 0.002447008 |
| 212515_s_at | 43 | 0.002449463 |
| 229692_at | 43 | 0.002449515 |
| 242024_at | 43 | 0.002453657 |
| 224811_at | 43 | 0.002458255 |
| 1557228_at | 43 | 0.002459728 |
| 234731_at | 43 | 0.002471747 |
| 1553349_at | 43 | 0.002482187 |
| 221836_s_at | 43 | 0.002482596 |
| 231247_s_at | 43 | 0.002484867 |
| 235613_at | 43 | 0.002496263 |
| 230127_at | 43 | 0.002505382 |
| 229590_at | 43 | 0.002513603 |
| 213788_s_at | 43 | 0.002514604 |
| 234949_at | 43 | 0.002515494 |
| 238712_at | 43 | 0.002518211 |
| 221704_s_at | 43 | 0.002524139 |
| 230395_at | 43 | 0.002524307 |
| 223986_x_at | 43 | 0.002529582 |
| 241427_x_at | 43 | 0.002534211 |
| 226286_at | 43 | 0.002538769 |
| 212981_s_at | 43 | 0.002541931 |
| 201744_s_at | 43 | 0.002551297 |
| 220777_at | 43 | 0.002556912 |
| 239264_at | 43 | 0.002557992 |
| 37226_at | 43 | 0.002558853 |
| 232546_at | 43 | 0.002558948 |
| 225334_at | 43 | 0.002567268 |
| 226921_at | 43 | 0.002572045 |
| 217448_s_at | 43 | 0.00257427 |
| 200651_at | 43 | 0.002580084 |
| 222662_at | 43 | 0.002587314 |
| 207669_at | 43 | 0.002588739 |
| 217939_s_at | 43 | 0.002597254 |
| 218437_s_at | 43 | 0.002600586 |
| 207396_s_at | 43 | 0.002604151 |
| 217820_s_at | 43 | 0.002607659 |
| 212847_at | 43 | 0.002611331 |
| 233867_at | 43 | 0.002614155 |
| 213946_s_at | 43 | 0.00261441 |
| 214785_at | 43 | 0.002614646 |
| 222141_at | 43 | 0.002622692 |
| 240098_at | 43 | 0.002651366 |
| 208696_at | 43 | 0.002655333 |
| 1558342_x_at | 43 | 0.002657132 |
| 223797_at | 43 | 0.002663774 |
| 241677_x_at | 43 | 0.002679773 |
| 215383_x_at | 43 | 0.002685438 |
| 232058_at | 43 | 0.002687121 |
| 224744_at | 43 | 0.002687395 |
| 234788_x_at | 43 | 0.002699861 |
| 238049_at | 43 | 0.002701412 |
| 230689_at | 43 | 0.002719337 |
| 212163_at | 43 | 0.002724202 |
| 233364_s_at | 43 | 0.002725864 |
| 242903_at | 43 | 0.002740387 |
| 1562020_s_at | 43 | 0.002748299 |
| 206128_at | 43 | 0.002749211 |
| 232523_at | 43 | 0.002752409 |
| 233223_at | 43 | 0.002760705 |
| 235804_at | 43 | 0.002781079 |
| 227544_at | 43 | 0.002782203 |
| 238883_at | 43 | 0.002785643 |
| 206940_s_at | 43 | 0.002789493 |
| 216471_x_at | 43 | 0.00279257 |
| 231057_at | 43 | 0.002793293 |
| 228758_at | 43 | 0.002796186 |
| 203156_at | 43 | 0.002801015 |
| 210541_s_at | 43 | 0.002803726 |
| 232908_at | 43 | 0.002818551 |
| 225975_at | 43 | 0.002826003 |
| 227126_at | 43 | 0.002826902 |
| 235155_at | 43 | 0.002829465 |
| 214060_at | 43 | 0.002829546 |
| 239408_at | 43 | 0.002840716 |
| 223261_at | 43 | 0.002850206 |
| 235592_at | 43 | 0.00287388 |
| 238619_at | 43 | 0.002878303 |
| 203114_at | 43 | 0.002880716 |
| 213708_s_at | 43 | 0.00288524 |
| 202777_at | 43 | 0.002897252 |
| 242297_at | 43 | 0.002902027 |
| 235063_at | 43 | 0.00290418 |
| 236869_at | 43 | 0.002904485 |
| 212944_at | 43 | 0.002916912 |
| 208612_at | 43 | 0.00292237 |
| 1563458_at | 43 | 0.00293355 |
| 238193_at | 43 | 0.002945978 |
| 238220_at | 43 | 0.002952318 |
| 241750_x_at | 43 | 0.002954968 |
| 217938_s_at | 43 | 0.002956477 |
| 219400_at | 43 | 0.002968997 |
| 225229_at | 43 | 0.00298479 |
| 227371_at | 43 | 0.002986207 |
| 239432_at | 43 | 0.002986316 |
| 241751_at | 43 | 0.002988459 |
| 200784_s_at | 43 | 0.002994847 |
| 216094_at | 43 | 0.002999304 |
| 229443_at | 43 | 0.003010578 |
| 200599_s_at | 43 | 0.003010589 |
| 216765_at | 43 | 0.003032732 |
| 239817_at | 43 | 0.0030407 |
| 237237_at | 43 | 0.003043115 |
| 209884_s_at | 43 | 0.003045073 |
| 203979_at | 43 | 0.003054066 |
| 222358_x_at | 43 | 0.003059534 |
| 242559_at | 43 | 0.003067174 |
| 243055_at | 43 | 0.003067288 |
| 232090_at | 43 | 0.003068213 |
| 224331_s_at | 43 | 0.003075251 |
| 240527_at | 43 | 0.003076633 |
| 212411_at | 43 | 0.003083644 |
| 233271_at | 43 | 0.003085337 |
| 221820_s_at | 43 | 0.003095798 |
| 213684_s_at | 43 | 0.00309736 |
| 225185_at | 43 | 0.003097769 |
| 1560500_at | 43 | 0.003100768 |
| 201613_s_at | 43 | 0.003105091 |
| 228905_at | 43 | 0.003108458 |
| 201683_x_at | 43 | 0.003130228 |
| 230397_at | 43 | 0.00313519 |
| 213880_at | 43 | 0.00314109 |
| 235026_at | 43 | 0.003151348 |
| 202580_x_at | 43 | 0.003178518 |
| 236886_at | 43 | 0.003179825 |
| 227568_at | 43 | 0.003181227 |
| 209893_s_at | 43 | 0.003182688 |
| 213202_at | 43 | 0.003187244 |
| 227143_s_at | 43 | 0.003206164 |
| 203513_at | 43 | 0.003217511 |
| 225653_at | 43 | 0.0032208 |
| 204123_at | 43 | 0.003234458 |
| 204091_at | 43 | 0.003237767 |
| 231987_at | 43 | 0.003240472 |
| 214093_s_at | 43 | 0.003248095 |
| 232480_at | 43 | 0.003249884 |
| 1561654_at | 43 | 0.003250675 |
| 241272_at | 43 | 0.003255536 |
| 242875_at | 43 | 0.003283009 |
| 236114_at | 43 | 0.003291526 |
| 219293_s_at | 43 | 0.003299798 |
| 218196_at | 43 | 0.003312597 |
| 236924_at | 43 | 0.003317181 |
| 212997_s_at | 43 | 0.00332128 |
| 236666_s_at | 43 | 0.00333788 |
| 242074_at | 43 | 0.003350338 |
| 229178_at | 43 | 0.003365467 |
| 210394_x_at | 43 | 0.003365982 |
| 228214_at | 43 | 0.003372297 |
| 227296_at | 43 | 0.003372683 |
| 213910_at | 43 | 0.00337473 |
| 200659_s_at | 43 | 0.003377242 |
| 214073_at | 43 | 0.003395441 |
| 226442_at | 43 | 0.003398594 |
| 239811_at | 43 | 0.003399481 |
| 209708_at | 43 | 0.003400614 |
| 1557707_at | 43 | 0.003414422 |
| 1561886_a_at | 43 | 0.00342105 |
| 242195_x_at | 43 | 0.003432118 |
| 202404_s_at | 43 | 0.003442945 |
| 240373_at | 43 | 0.003447895 |
| 213319_s_at | 43 | 0.003464694 |
| 201031_s_at | 43 | 0.003466322 |
| 1552803_a_at | 43 | 0.00346752 |
| 221042_s_at | 43 | 0.003484682 |
| 202561_at | 43 | 0.003485942 |
| 218841_at | 43 | 0.003493244 |
| 238852_at | 43 | 0.00349511 |
| 203150_at | 43 | 0.003505531 |
| 230843_at | 43 | 0.003527222 |
| 240307_at | 43 | 0.003541753 |
| 211561_x_at | 43 | 0.003545036 |
| 231087_at | 43 | 0.003546674 |
| 221478_at | 43 | 0.003561987 |
| 1557541_at | 43 | 0.003570285 |
| 221094_s_at | 43 | 0.003570907 |
| 200785_s_at | 43 | 0.003571339 |
| 218146_at | 43 | 0.003571563 |
| 239469_at | 43 | 0.003573441 |
| 1561355_at | 43 | 0.003578338 |
| 232544_at | 43 | 0.003579825 |
| 227183_at | 43 | 0.003587571 |
| 223238_s_at | 43 | 0.003595533 |
| 212668_at | 43 | 0.003602767 |
| 200689_x_at | 43 | 0.003619946 |
| 210915_x_at | 43 | 0.003629235 |
| 227842_at | 43 | 0.003629772 |
| 1560172_at | 43 | 0.003632272 |
| 203113_s_at | 43 | 0.003646878 |
| 219919_s_at | 43 | 0.003651025 |
| 204413_at | 43 | 0.003651758 |
| 228024_at | 43 | 0.003654476 |
| 205831_at | 43 | 0.003656931 |
| 208023_at | 43 | 0.003659276 |
| 224656_s_at | 43 | 0.003661527 |
| 235652_at | 43 | 0.003663015 |
| 232525_at | 43 | 0.00366741 |
| 233228_at | 43 | 0.003685105 |
| 227497_at | 43 | 0.0036876 |
| 207046_at | 43 | 0.003692534 |
| 238199_x_at | 43 | 0.00369434 |
| 244858_at | 43 | 0.003699841 |
| 203330_s_at | 43 | 0.003700506 |
| 226146_at | 43 | 0.003706873 |
| 206796_at | 43 | 0.003710696 |
| 234948_at | 43 | 0.003712026 |
| 228090_at | 43 | 0.003712445 |
| 235197_s_at | 43 | 0.003745183 |
| 222282_at | 43 | 0.003747972 |
| 1563455_at | 43 | 0.003759716 |
| 212136_at | 43 | 0.003761193 |
| 225949_at | 43 | 0.003769417 |
| 222236_s_at | 43 | 0.003778511 |
| 202351_at | 43 | 0.003783746 |
| 242405_at | 43 | 0.003783969 |
| 206746_at | 43 | 0.003785601 |
| 217447_at | 43 | 0.003792079 |
| 227899_at | 43 | 0.00381253 |
| 208129_x_at | 43 | 0.003817668 |
| 225338_at | 43 | 0.003817815 |
| 201500_s_at | 43 | 0.003818357 |
| 206179_s_at | 43 | 0.003823478 |
| 227536_at | 43 | 0.003832363 |
| 201687_s_at | 43 | 0.003837246 |
| 1558847_at | 43 | 0.003838883 |
| 202827_s_at | 43 | 0.003864151 |
| 204320_at | 43 | 0.003864379 |
| 1555269_a_at | 43 | 0.003870171 |
| 223662_x_at | 43 | 0.003871572 |
| 218860_at | 43 | 0.003879251 |
| 244292_at | 43 | 0.003882132 |
| 232030_at | 43 | 0.003890329 |
| 209739_s_at | 43 | 0.00389284 |
| 235699_at | 43 | 0.003896227 |
| 1553193_at | 43 | 0.003901363 |
| 223815_at | 43 | 0.003905517 |
| 226240_at | 43 | 0.003927621 |
| 1558116_x_at | 43 | 0.003930113 |
| 215515_at | 43 | 0.00393623 |
| 222814_s_at | 43 | 0.003940144 |
| 241793_at | 43 | 0.003946339 |
| 200851_s_at | 43 | 0.003947063 |
| 231914_at | 43 | 0.003949602 |
| 224448_s_at | 43 | 0.00395416 |
| 221256_s_at | 43 | 0.003956147 |
| 212191_x_at | 43 | 0.003964675 |
| 202034_x_at | 43 | 0.00396474 |
| 212900_at | 43 | 0.003965649 |
| 202766_s_at | 43 | 0.003976771 |
| 222732_at | 43 | 0.004008513 |
| 236072_at | 43 | 0.004014929 |
| 223411_at | 43 | 0.004015931 |
| 1569114_at | 43 | 0.004020966 |
| 228432_at | 43 | 0.0040271 |
| 209161_at | 43 | 0.004028299 |
| 213131_at | 43 | 0.004040857 |
| 203794_at | 43 | 0.004045176 |
| 222525_s_at | 43 | 0.004045224 |
| 223808_s_at | 43 | 0.004046944 |
| 222436_s_at | 43 | 0.004050752 |
| 240467_at | 43 | 0.004051165 |
| 232235_at | 43 | 0.004052375 |
| 241905_at | 43 | 0.00406109 |
| 211425_x_at | 43 | 0.004066763 |
| 242472_x_at | 43 | 0.00407264 |
| 201295_s_at | 43 | 0.004077595 |
| 212982_at | 43 | 0.004078366 |
| 204460_s_at | 43 | 0.004081296 |
| 229431_at | 43 | 0.004087245 |
| 200997_at | 43 | 0.004089019 |
| 1569167_at | 43 | 0.00409005 |
| 202822_at | 43 | 0.00409018 |
| 232890_at | 43 | 0.004090836 |
| 236696_at | 43 | 0.004093975 |
| 212467_at | 43 | 0.004112691 |
| 208669_s_at | 43 | 0.004117947 |
| 213085_s_at | 43 | 0.004130043 |
| 210059_s_at | 43 | 0.004130482 |
| 240108_at | 43 | 0.004131108 |
| 227172_at | 43 | 0.004132086 |
| 228789_at | 43 | 0.004134296 |
| 240410_at | 43 | 0.004137164 |
| 219700_at | 43 | 0.00414216 |
| 236215_at | 43 | 0.004143897 |
| 214297_at | 43 | 0.004146451 |
| 230361_at | 43 | 0.004153507 |
| 227597_at | 43 | 0.004160173 |
| 227767_at | 43 | 0.004166097 |
| 233055_at | 43 | 0.00416986 |
| 215855_s_at | 43 | 0.004169979 |
| 203261_at | 43 | 0.004180606 |
| 244197_x_at | 43 | 0.00418829 |
| 238082_at | 43 | 0.00421052 |
| 208929_x_at | 43 | 0.004217231 |
| 65133_i_at | 43 | 0.004218771 |
| 233109_at | 43 | 0.004222382 |
| 201160_s_at | 43 | 0.004239494 |
| 232455_x_at | 43 | 0.004267128 |
| 233152_x_at | 43 | 0.00427709 |
| 240326_at | 43 | 0.004278177 |
| 214046_at | 43 | 0.004286093 |
| 238890_at | 43 | 0.004287036 |
| 227915_at | 43 | 0.004290795 |
| 215095_at | 43 | 0.004291903 |
| 243690_at | 43 | 0.004292765 |
| 209832_s_at | 43 | 0.004318294 |
| 235664_at | 43 | 0.004321659 |
| 1555068_at | 43 | 0.004331635 |
| 221043_at | 43 | 0.004340556 |
| 228088_at | 43 | 0.004352064 |
| 227333_at | 43 | 0.004352442 |
| 204630_s_at | 43 | 0.004356354 |
| 1562957_at | 43 | 0.0043566 |
| 225760_at | 43 | 0.00435843 |
| 228414_at | 43 | 0.004369234 |
| 238987_at | 43 | 0.004380702 |
| 224336_s_at | 43 | 0.004399024 |
| 234049_at | 43 | 0.004409292 |
| 238908_at | 43 | 0.004416702 |
| 217279_x_at | 43 | 0.004416764 |
| 215310_at | 43 | 0.004416956 |
| 235723_at | 43 | 0.004419982 |
| 226318_at | 43 | 0.004421112 |
| 201459_at | 43 | 0.004433776 |
| 1568617_a_at | 43 | 0.004442614 |
| 205981_s_at | 43 | 0.004445892 |
| 223245_at | 43 | 0.004446934 |
| 216230_x_at | 43 | 0.004451558 |
| 214527_s_at | 43 | 0.004456481 |
| 242506_at | 43 | 0.004467154 |
| 234343_s_at | 43 | 0.004478132 |
| 224700_at | 43 | 0.004485036 |
| 200771_at | 43 | 0.004485195 |
| 202697_at | 43 | 0.004493081 |
| 213773_x_at | 43 | 0.004504494 |
| 215204_at | 43 | 0.004505962 |
| 1557240_a_at | 43 | 0.004508945 |
| 209315_at | 43 | 0.004515409 |
| 224883_at | 43 | 0.004521883 |
| 219420_s_at | 43 | 0.004536594 |
| 213605_s_at | 43 | 0.004537355 |
| 211214_s_at | 43 | 0.004541045 |
| 205621_at | 43 | 0.004554073 |
| 221069_s_at | 43 | 0.004557806 |
| 211891_s_at | 43 | 0.004559454 |
| 232351_at | 43 | 0.004562236 |
| 202645_s_at | 43 | 0.004571163 |
| 226584_s_at | 43 | 0.004575657 |
| 1565639_a_at | 43 | 0.004583261 |
| 213213_at | 43 | 0.004589667 |
| 228980_at | 43 | 0.004590218 |
| 220320_at | 43 | 0.004602965 |
| 219879_s_at | 43 | 0.004612721 |
| 209202_s_at | 43 | 0.004615813 |
| 221920_s_at | 43 | 0.004628971 |
| 222313_at | 43 | 0.004639992 |
| 212084_at | 43 | 0.004640638 |
| 219245_s_at | 43 | 0.004640651 |
| 203458_at | 43 | 0.00464136 |
| 235253_at | 43 | 0.004641434 |
| 202176_at | 43 | 0.004645903 |
| 233333_x_at | 43 | 0.004647332 |
| 1553301_a_at | 43 | 0.004674542 |
| 227639_at | 43 | 0.004677095 |
| 244069_at | 43 | 0.004679132 |
| 221973_at | 43 | 0.004680285 |
| 228315_at | 43 | 0.004688141 |
| 241769_at | 43 | 0.004712495 |
| 226277_at | 43 | 0.004720272 |
| 201560_at | 43 | 0.004723174 |
| 239901_at | 43 | 0.004728161 |
| 218644_at | 43 | 0.004731305 |
| 228846_at | 43 | 0.004732739 |
| 234645_at | 43 | 0.004740334 |
| 201161_s_at | 43 | 0.004751137 |
| 1557675_at | 43 | 0.004751288 |
| 229535_at | 43 | 0.004751401 |
| 205768_s_at | 43 | 0.004751741 |
| 221919_at | 43 | 0.004755878 |
| 208363_s_at | 43 | 0.004760205 |
| 202461_at | 43 | 0.004773174 |
| 225827_at | 43 | 0.004775146 |
| 47571_at | 43 | 0.004775446 |
| 1558448_a_at | 43 | 0.00477863 |
| 229666_s_at | 43 | 0.004782532 |
| 222773_s_at | 43 | 0.004788469 |
| 227940_at | 43 | 0.004796763 |
| 205780_at | 43 | 0.004797632 |
| 1564520_s_at | 43 | 0.004821589 |
| 218907_s_at | 43 | 0.004822509 |
| 200863_s_at | 43 | 0.004827748 |
| 211345_x_at | 43 | 0.004846309 |
| 229761_at | 43 | 0.004851565 |
| 208606_s_at | 43 | 0.004853903 |
| 236148_at | 43 | 0.004855004 |
| 220468_at | 43 | 0.004857353 |
| 215199_at | 43 | 0.004858565 |
| 244425_at | 43 | 0.004884436 |
| 216028_at | 43 | 0.004888763 |
| 1560031_at | 43 | 0.004900381 |
| 223308_s_at | 43 | 0.004914391 |
| 235584_at | 43 | 0.004917093 |
| 202703_at | 43 | 0.004931138 |
| 1559946_s_at | 43 | 0.004931939 |
| 236524_at | 43 | 0.004946623 |
| 214949_at | 43 | 0.004950313 |
| 230958_s_at | 43 | 0.004951007 |
| 215188_at | 43 | 0.004955362 |
| 202264_s_at | 43 | 0.004959527 |
| 213122_at | 43 | 0.004973541 |
| 223580_at | 43 | 0.004973861 |
| 209516_at | 43 | 0.004978683 |
| 210502_s_at | 43 | 0.004980675 |
| 238504_at | 43 | 0.004983982 |
| 218137_s_at | 43 | 0.00498893 |
| 201580_s_at | 43 | 0.004989557 |
| 244503_at | 43 | 0.004998675 |
| 242413_at | 43 | 0.005001232 |
| 232322_x_at | 43 | 0.005011445 |
| 205854_at | 43 | 0.005014419 |
| 200097_s_at | 43 | 0.005016799 |
| 228271_at | 43 | 0.005024014 |
| 1565701_at | 43 | 0.005024034 |
| 212986_s_at | 43 | 0.00502728 |
| 235000_at | 43 | 0.005031073 |
| 218267_at | 43 | 0.005032206 |
| 225523_at | 43 | 0.00503614 |
| 203274_at | 43 | 0.005039836 |
| 213445_at | 43 | 0.005046431 |
| 205836_s_at | 43 | 0.005048785 |
| 229549_at | 43 | 0.005053673 |
| 236957_at | 43 | 0.00506071 |
| 233889_at | 43 | 0.005066303 |
| 215560_x_at | 43 | 0.005067728 |
| 209316_s_at | 43 | 0.005067823 |
| 1562442_a_at | 43 | 0.005074165 |
| 223293_at | 43 | 0.005081525 |
| 226484_at | 43 | 0.005082166 |
| 219660_s_at | 43 | 0.005082446 |
| 203884_s_at | 43 | 0.005085986 |
| 230742_at | 43 | 0.005094015 |
| 202535_at | 43 | 0.005099349 |
| 233411_at | 43 | 0.005103537 |
| 221768_at | 43 | 0.005113704 |
| 205224_at | 43 | 0.005119024 |
| 243286_at | 43 | 0.005119257 |
| 235376_at | 43 | 0.005147581 |
| 222792_s_at | 43 | 0.005152188 |
| 232975_at | 43 | 0.00516156 |
| 213686_at | 43 | 0.005162155 |
| 229226_at | 43 | 0.005186698 |
| 1556339_a_at | 43 | 0.00518765 |
| 235890_at | 43 | 0.005192432 |
| 1564151_at | 43 | 0.005206389 |
| 1553346_a_at | 43 | 0.005210616 |
| 1558467_a_at | 43 | 0.00521763 |
| 242619_x_at | 43 | 0.005226401 |
| 227455_at | 43 | 0.005230759 |
| 218187_s_at | 43 | 0.005238572 |
| 221065_s_at | 43 | 0.00524999 |
| 200827_at | 43 | 0.005259525 |
| 223055_s_at | 43 | 0.005261007 |
| 1555960_at | 43 | 0.005280745 |
| 236974_at | 43 | 0.005283107 |
| 224643_at | 43 | 0.005289232 |
| 235193_at | 43 | 0.005294699 |
| 229424_s_at | 43 | 0.005300538 |
| 1560754_at | 43 | 0.005306123 |
| 209717_at | 43 | 0.005320096 |
| 237306_at | 43 | 0.005326683 |
| 233262_at | 43 | 0.005341986 |
| 215114_at | 43 | 0.005351434 |
| 203608_at | 43 | 0.005353823 |
| 235287_at | 43 | 0.005395153 |
| 239784_at | 43 | 0.005398234 |
| 239777_at | 43 | 0.005403014 |
| 204055_s_at | 43 | 0.005406013 |
| 240400_at | 43 | 0.005411068 |
| 204461_x_at | 43 | 0.005414553 |
| 242609_x_at | 43 | 0.00542712 |
| 216278_at | 43 | 0.00544225 |
| 227456_s_at | 43 | 0.005448067 |
| 223574_x_at | 43 | 0.005454064 |
| 213193_x_at | 43 | 0.005458219 |
| 201046_s_at | 43 | 0.005458781 |
| 225442_at | 43 | 0.005461692 |
| 1558748_at | 43 | 0.005463844 |
| 218989_x_at | 43 | 0.005495609 |
| 218348_s_at | 43 | 0.005499898 |
| 209295_at | 43 | 0.0055026 |
| 242593_at | 43 | 0.005506674 |
| 213005_s_at | 43 | 0.005507072 |
| 241907_at | 43 | 0.005516291 |
| 221257_x_at | 43 | 0.005524257 |
| 240830_at | 43 | 0.005528855 |
| 221754_s_at | 43 | 0.005530536 |
| 242167_at | 43 | 0.005540516 |
| 205769_at | 43 | 0.005546453 |
| 214724_at | 43 | 0.005546513 |
| 223484_at | 43 | 0.005578061 |
| 1557050_at | 43 | 0.00558046 |
| 201673_s_at | 43 | 0.005587544 |
| 52255_s_at | 43 | 0.005588279 |
| 209927_s_at | 43 | 0.005593909 |
| 242191_at | 43 | 0.005603698 |
| 236947_at | 43 | 0.005606793 |
| 217829_s_at | 43 | 0.005607188 |
| 239550_at | 43 | 0.005609971 |
| 211686_s_at | 43 | 0.005627384 |
| 239409_at | 43 | 0.005627938 |
| 201891_s_at | 43 | 0.005630598 |
| 227541_at | 43 | 0.005634931 |
| 1559901_s_at | 43 | 0.005635588 |
| 224100_s_at | 43 | 0.005649066 |
| 201600_at | 43 | 0.005653871 |
| 228010_at | 43 | 0.005656578 |
| 208758_at | 43 | 0.005662094 |
| 226639_at | 43 | 0.005675268 |
| 235871_at | 43 | 0.005695324 |
| 210552_s_at | 43 | 0.005702253 |
| 238878_at | 43 | 0.005710548 |
| 243170_at | 43 | 0.005716687 |
| 244441_at | 43 | 0.00571946 |
| 238966_at | 43 | 0.005733886 |
| 224566_at | 43 | 0.005736239 |
| 235535_x_at | 43 | 0.005741927 |
| 221899_at | 43 | 0.005745314 |
| 203883_s_at | 43 | 0.005765565 |
| 218804_at | 43 | 0.005775151 |
| 222703_s_at | 43 | 0.005777458 |
| 226499_at | 43 | 0.005786177 |
| 211087_x_at | 43 | 0.005791638 |
| 235060_at | 43 | 0.005794949 |
| 210216_x_at | 43 | 0.005829913 |
| 230503_at | 43 | 0.005833397 |
| 222998_at | 43 | 0.00584109 |
| 212734_x_at | 43 | 0.00584528 |
| 209147_s_at | 43 | 0.005845528 |
| 216977_x_at | 43 | 0.005858273 |
| 236862_at | 43 | 0.005866596 |
| 229733_s_at | 43 | 0.005871464 |
| 203094_at | 43 | 0.005881558 |
| 233768_at | 43 | 0.00588478 |
| 242853_at | 43 | 0.005896633 |
| 212877_at | 43 | 0.00591022 |
| 200684_s_at | 43 | 0.005911273 |
| 242233_at | 43 | 0.005921494 |
| 241464_s_at | 43 | 0.005932809 |
| 232528_at | 43 | 0.005941639 |
| 203866_at | 43 | 0.005954023 |
| 204936_at | 43 | 0.005956273 |
| 205599_at | 43 | 0.005957565 |
| 238009_at | 43 | 0.006029191 |
| 217659_at | 43 | 0.006029579 |
| 1558688_at | 43 | 0.006032074 |
| 201827_at | 43 | 0.006033642 |
| 225434_at | 43 | 0.006034581 |
| 238894_at | 43 | 0.006047593 |
| 234762_x_at | 43 | 0.006048555 |
| 218928_s_at | 43 | 0.006051155 |
| 204868_at | 43 | 0.006061485 |
| 232023_at | 43 | 0.00606743 |
| 230728_at | 43 | 0.00607242 |
| 205093_at | 43 | 0.006074603 |
| 227041_at | 43 | 0.006077373 |
| 227561_at | 43 | 0.006087358 |
| 228618_at | 43 | 0.006096899 |
| 229319_at | 43 | 0.006114075 |
| 241865_at | 43 | 0.006114139 |
| 204493_at | 43 | 0.006118029 |
| 236795_at | 43 | 0.006124848 |
| 211113_s_at | 43 | 0.006132453 |
| 233473_x_at | 43 | 0.006136489 |
| 203253_s_at | 43 | 0.006140151 |
| 241713_s_at | 43 | 0.006141461 |
| 233400_at | 43 | 0.006143553 |
| 235879_at | 43 | 0.006144787 |
| 201256_at | 43 | 0.006150908 |
| 206071_s_at | 43 | 0.006167701 |
| 216682_s_at | 43 | 0.006167994 |
| 223779_at | 43 | 0.006182542 |
| 223062_s_at | 43 | 0.006183722 |
| 227761_at | 43 | 0.006189263 |
| 233557_s_at | 43 | 0.006195137 |
| 238683_at | 43 | 0.006196047 |
| 239311_at | 43 | 0.006197816 |
| 225879_at | 43 | 0.006199464 |
| 1560145_at | 43 | 0.006208797 |
| 236962_at | 43 | 0.006208802 |
| 228463_at | 43 | 0.006211096 |
| 205881_at | 43 | 0.006222124 |
| 1554703_at | 43 | 0.006234018 |
| 222094_at | 43 | 0.006257729 |
| 1563010_at | 43 | 0.006264454 |
| 235619_at | 43 | 0.006270339 |
| 1559352_a_at | 43 | 0.006287368 |
| 1559154_at | 43 | 0.006289296 |
| 221833_at | 43 | 0.006293115 |
| 204545_at | 43 | 0.006300811 |
| 238431_at | 43 | 0.006302216 |
| 1557578_at | 43 | 0.006302618 |
| 1569020_at | 43 | 0.006304674 |
| 211796_s_at | 43 | 0.006322102 |
| 227579_at | 43 | 0.006324227 |
| 242982_x_at | 43 | 0.006325461 |
| 238448_at | 43 | 0.006333456 |
| 205168_at | 43 | 0.006338354 |
| 239379_at | 43 | 0.006350187 |
| 220558_x_at | 43 | 0.006352686 |
| 231873_at | 43 | 0.006369668 |
| 1568643_a_at | 43 | 0.006379264 |
| 229235_at | 43 | 0.006379619 |
| 236619_at | 43 | 0.006380561 |
| 229573_at | 43 | 0.006388376 |
| 226968_at | 43 | 0.006398876 |
| 211927_x_at | 43 | 0.006399905 |
| 211936_at | 43 | 0.006428951 |
| 235985_at | 43 | 0.006441161 |
| 233140_s_at | 43 | 0.006453743 |
| 242837_at | 43 | 0.006455486 |
| 232432_s_at | 43 | 0.006465308 |
| 235078_at | 43 | 0.006466482 |
| 211841_s_at | 43 | 0.006467117 |
| 221520_s_at | 43 | 0.006468491 |
| 211987_at | 43 | 0.006476806 |
| 217598_at | 43 | 0.006477408 |
| 228009_x_at | 43 | 0.006479126 |
| 216596_at | 43 | 0.006483983 |
| 1557046_x_at | 43 | 0.006488732 |
| 208685_x_at | 43 | 0.006494512 |
| 209955_s_at | 43 | 0.006496058 |
| 213111_at | 43 | 0.006497239 |
| 241630_at | 43 | 0.006503573 |
| 208670_s_at | 43 | 0.006517059 |
| 227191_at | 43 | 0.006519885 |
| 233857_s_at | 43 | 0.00652354 |
| 214911_s_at | 43 | 0.006528125 |
| 219411_at | 43 | 0.006530323 |
| 236598_at | 43 | 0.006532558 |
| 209430_at | 43 | 0.006546846 |
| 221052_at | 43 | 0.006553881 |
| 212872_s_at | 43 | 0.006555782 |
| 203433_at | 43 | 0.006562281 |
| 201719_s_at | 43 | 0.006564002 |
| 229942_at | 43 | 0.006569687 |
| 232473_at | 43 | 0.006571337 |
| 1569703_a_at | 43 | 0.006574117 |
| 202221_s_at | 43 | 0.006575245 |
| 223682_s_at | 43 | 0.006584885 |
| 224691_at | 43 | 0.006614333 |
| 208128_x_at | 43 | 0.006618808 |
| 223825_at | 43 | 0.006619288 |
| 225253_s_at | 43 | 0.006639651 |
| 223018_at | 43 | 0.006652465 |
| 216449_x_at | 43 | 0.006657839 |
| 216038_x_at | 43 | 0.006671461 |
| 219162_s_at | 43 | 0.006677901 |
| 209162_s_at | 43 | 0.006681929 |
| 236967_at | 43 | 0.006682154 |
| 236288_at | 43 | 0.006684326 |
| 201395_at | 43 | 0.00670726 |
| 201210_at | 43 | 0.006708514 |
| 212690_at | 43 | 0.006720671 |
| 204937_s_at | 43 | 0.006724303 |
| 1569067_at | 43 | 0.006725249 |
| 1556676_a_at | 43 | 0.006725311 |
| 219185_at | 43 | 0.006738928 |
| 206508_at | 43 | 0.006751547 |
| 1555938_x_at | 43 | 0.006753053 |
| 241632_x_at | 43 | 0.006757591 |
| 228045_at | 43 | 0.006763451 |
| 229346_at | 43 | 0.006766056 |
| 1564424_at | 43 | 0.006775251 |
| 227335_at | 43 | 0.006799922 |
| 201298_s_at | 43 | 0.006813648 |
| 204736_s_at | 43 | 0.006819886 |
| 224474_x_at | 43 | 0.006827889 |
| 242627_at | 43 | 0.006843049 |
| 212249_at | 43 | 0.006846574 |
| 202765_s_at | 43 | 0.006860632 |
| 241838_at | 43 | 0.00686865 |
| 1556744_a_at | 43 | 0.006870074 |
| 232307_at | 43 | 0.006883296 |
| 240246_at | 43 | 0.006907754 |
| 222376_at | 43 | 0.006909299 |
| 233254_x_at | 43 | 0.006909925 |
| 200051_at | 43 | 0.006913538 |
| 212701_at | 43 | 0.006917917 |
| 205667_at | 43 | 0.006931665 |
| 244753_at | 43 | 0.006935034 |
| 229577_at | 43 | 0.006940663 |
| 1560680_at | 43 | 0.00694415 |
| 226695_at | 43 | 0.006957455 |
| 210320_s_at | 43 | 0.006981477 |
| 223874_at | 43 | 0.00701749 |
| 201440_at | 43 | 0.007044338 |
| 215155_at | 43 | 0.007057713 |
| 241242_at | 43 | 0.007059467 |
| 218708_at | 43 | 0.007071542 |
| 241797_at | 43 | 0.007091205 |
| 1568853_at | 43 | 0.007092244 |
| 203688_at | 43 | 0.007098323 |
| 215203_at | 43 | 0.007098417 |
| 237768_x_at | 43 | 0.007100607 |
| 200755_s_at | 43 | 0.00711331 |
| 233219_at | 43 | 0.007113501 |
| 239763_at | 43 | 0.0071188 |
| 203103_s_at | 43 | 0.007131642 |
| 229981_at | 43 | 0.007135337 |
| 1564521_x_at | 43 | 0.007151144 |
| 226831_at | 43 | 0.007154741 |
| 234657_at | 43 | 0.007158897 |
| 233874_at | 43 | 0.007160293 |
| 209961_s_at | 43 | 0.007163973 |
| 235678_at | 43 | 0.007165613 |
| 205771_s_at | 43 | 0.007165808 |
| 226166_x_at | 43 | 0.007173033 |
| 224785_at | 43 | 0.007175762 |
| 200046_at | 43 | 0.007177519 |
| 234148_at | 43 | 0.007178172 |
| 214852_x_at | 43 | 0.007186074 |
| 223509_at | 43 | 0.007194348 |
| 232573_at | 43 | 0.007194568 |
| 232889_at | 43 | 0.007197097 |
| 235067_at | 43 | 0.007200443 |
| 215412_x_at | 43 | 0.007211158 |
| 210058_at | 43 | 0.007218178 |
| 205053_at | 43 | 0.007241575 |
| 200846_s_at | 43 | 0.007241725 |
| 213460_x_at | 43 | 0.007242588 |
| 200007_at | 43 | 0.007244827 |
| 209517_s_at | 43 | 0.007247258 |
| 242878_at | 43 | 0.007250098 |
| 201407_s_at | 43 | 0.007277576 |
| 215075_s_at | 43 | 0.007288329 |
| 201375_s_at | 43 | 0.007288657 |
| 202396_at | 43 | 0.007307625 |
| 210822_at | 43 | 0.007321439 |
| 223663_at | 43 | 0.007325042 |
| 228676_at | 43 | 0.007344044 |
| 232180_at | 43 | 0.007351774 |
| 224695_at | 43 | 0.007360183 |
| 223203_at | 43 | 0.007362087 |
| 236566_at | 43 | 0.007376964 |
| 214917_at | 43 | 0.007399896 |
| 218630_at | 43 | 0.007412462 |
| 212378_at | 43 | 0.007444123 |
| 218233_s_at | 43 | 0.007448659 |
| 233976_at | 43 | 0.007450827 |
| 219150_s_at | 43 | 0.007452917 |
| 203942_s_at | 43 | 0.007454421 |
| 203622_s_at | 43 | 0.00746076 |
| 233870_at | 43 | 0.007469607 |
| 220185_at | 43 | 0.007470419 |
| 243736_at | 43 | 0.007477228 |
| 242932_at | 43 | 0.007480098 |
| 202403_s_at | 43 | 0.007480993 |
| 229966_at | 43 | 0.007481402 |
| 205571_at | 43 | 0.007490658 |
| 233440_at | 43 | 0.007500823 |
| 209467_s_at | 43 | 0.00750195 |
| 232007_at | 43 | 0.007538481 |
| 217969_at | 43 | 0.007545807 |
| 1557252_at | 43 | 0.007559949 |
| 224966_s_at | 43 | 0.007561367 |
| 239892_at | 43 | 0.00756575 |
| 216933_x_at | 43 | 0.007567108 |
| 226619_at | 43 | 0.007574767 |
| 206683_at | 43 | 0.007580449 |
| 242222_at | 43 | 0.007584907 |
| 229969_at | 43 | 0.007593315 |
| 212563_at | 43 | 0.007599345 |
| 227498_at | 43 | 0.007603498 |
| 227682_at | 43 | 0.007614163 |
| 208620_at | 43 | 0.007618624 |
| 213962_s_at | 43 | 0.007619354 |
| 241588_at | 43 | 0.007652222 |
| 228471_at | 43 | 0.007661868 |
| 213300_at | 43 | 0.007664415 |
| 35148_at | 43 | 0.007665715 |
| 233321_x_at | 43 | 0.007684043 |
| 218383_at | 43 | 0.007687244 |
| 215828_at | 43 | 0.007687841 |
| 203755_at | 43 | 0.007698639 |
| 239567_at | 43 | 0.007702163 |
| 1570507_at | 43 | 0.007708484 |
| 233442_at | 43 | 0.007710726 |
| 227156_at | 43 | 0.007710884 |
| 227008_at | 43 | 0.00771134 |
| 1556543_at | 43 | 0.007713789 |
| 224952_at | 43 | 0.007721491 |
| 236462_at | 43 | 0.00772357 |
| 234005_x_at | 43 | 0.007725705 |
| 212454_x_at | 43 | 0.007742406 |
| 235103_at | 43 | 0.007758372 |
| 210963_s_at | 43 | 0.007763216 |
| 225219_at | 43 | 0.007772453 |
| 221821_s_at | 43 | 0.007781922 |
| 210789_x_at | 43 | 0.007796096 |
| 235940_at | 43 | 0.007798767 |
| 1554938_a_at | 43 | 0.00780428 |
| 221598_s_at | 43 | 0.007815793 |
| 207281_x_at | 43 | 0.007827708 |
| 216170_at | 43 | 0.007831579 |
| 241337_at | 43 | 0.007835986 |
| 205547_s_at | 43 | 0.007842158 |
| 205205_at | 43 | 0.007850798 |
| 215470_at | 43 | 0.007850982 |
| 229104_s_at | 43 | 0.007853386 |
| 232784_at | 43 | 0.007858171 |
| 1552286_at | 43 | 0.007866813 |
| 225070_at | 43 | 0.007866823 |
| 228479_at | 43 | 0.007869948 |
| 243966_at | 43 | 0.007878485 |
| 224882_at | 43 | 0.007884966 |
| 222220_s_at | 43 | 0.007886304 |
| 235158_at | 43 | 0.007896895 |
| 228057_at | 43 | 0.007897573 |
| 229291_at | 43 | 0.007918778 |
| 206268_at | 43 | 0.007925193 |
| 205407_at | 43 | 0.007925291 |
| 1569377_at | 43 | 0.007929659 |
| 228462_at | 43 | 0.007934227 |
| 36865_at | 43 | 0.007945764 |
| 232923_at | 43 | 0.007947487 |
| 212685_s_at | 43 | 0.00795627 |
| 1565149_at | 43 | 0.007962339 |
| 200019_s_at | 43 | 0.007970122 |
| 223156_at | 43 | 0.007975871 |
| 223631_s_at | 43 | 0.007976137 |
| 204294_at | 43 | 0.007990322 |
| 215305_at | 43 | 0.008004992 |
| 209496_at | 43 | 0.008006217 |
| 208848_at | 43 | 0.008016437 |
| 212204_at | 43 | 0.008033211 |
| 217408_at | 43 | 0.008038178 |
| 213618_at | 43 | 0.008042608 |
| 1556769_a_at | 43 | 0.008044038 |
| 233296_x_at | 43 | 0.008044303 |
| 219378_at | 43 | 0.008061799 |
| 231806_s_at | 43 | 0.008064323 |
| 215898_at | 43 | 0.00806444 |
| 205259_at | 43 | 0.008091183 |
| 205037_at | 43 | 0.008101629 |
| 240324_at | 43 | 0.008108946 |
| 244579_at | 43 | 0.008109266 |
| 230353_at | 43 | 0.008113566 |
| 232458_at | 43 | 0.008115827 |
| 202275_at | 43 | 0.008140317 |
| 236859_at | 43 | 0.008146683 |
| 1566231_at | 43 | 0.008148562 |
| 238303_at | 43 | 0.008153719 |
| 209936_at | 43 | 0.008168783 |
| 243498_at | 43 | 0.008187472 |
| 242611_at | 43 | 0.008206651 |
| 223506_at | 43 | 0.008225761 |
| 202161_at | 43 | 0.008227118 |
| 213913_s_at | 43 | 0.008235907 |
| 210165_at | 43 | 0.008246309 |
| 242607_at | 43 | 0.008248549 |
| 214048_at | 43 | 0.008255864 |
| 225814_at | 43 | 0.008257352 |
| 209785_s_at | 43 | 0.008260342 |
| 201400_at | 43 | 0.008274005 |
| 235585_at | 43 | 0.008279009 |
| 229694_at | 43 | 0.008279243 |
| 233690_at | 43 | 0.008283543 |
| 213136_at | 43 | 0.008295346 |
| 219103_at | 43 | 0.00830002 |
| 236012_at | 43 | 0.008308755 |
| 206056_x_at | 43 | 0.008317453 |
| 229933_at | 43 | 0.008318735 |
| 241152_at | 43 | 0.008319539 |
| 202923_s_at | 43 | 0.008362153 |
| 232333_at | 43 | 0.008374129 |
| 1560275_at | 43 | 0.008384716 |
| 233995_at | 43 | 0.008411746 |
| 200816_s_at | 43 | 0.008411973 |
| 239005_at | 43 | 0.00841509 |
| 1559140_at | 43 | 0.008420022 |
| 244356_at | 43 | 0.008437117 |
| 242440_at | 43 | 0.008442907 |
| 1557505_a_at | 43 | 0.008447715 |
| 235373_at | 43 | 0.00844953 |
| 204465_s_at | 43 | 0.008457195 |
| 237362_at | 43 | 0.008460999 |
| 1555724_s_at | 43 | 0.008461604 |
| 33850_at | 43 | 0.008466435 |
| 244648_at | 43 | 0.008484721 |
| 229901_at | 43 | 0.008492719 |
| 226479_at | 43 | 0.008495457 |
| 219203_at | 43 | 0.008498145 |
| 1557580_at | 43 | 0.008499799 |
| 225315_at | 43 | 0.008505842 |
| 203525_s_at | 43 | 0.008507487 |
| 224783_at | 43 | 0.00850917 |
| 214989_x_at | 43 | 0.008523781 |
| 208082_x_at | 43 | 0.008526243 |
| 239373_at | 43 | 0.008545163 |
| 232778_at | 43 | 0.00855251 |
| 1557812_a_at | 43 | 0.008560579 |
| 209008_x_at | 43 | 0.008562692 |
| 239561_at | 43 | 0.008587024 |
| 222167_at | 43 | 0.008591656 |
| 51192_at | 43 | 0.008592752 |
| 242422_at | 43 | 0.008628695 |
| 229156_s_at | 43 | 0.008629098 |
| 225406_at | 43 | 0.008633219 |
| 201581_at | 43 | 0.008638602 |
| 243856_at | 43 | 0.008645292 |
| 242645_at | 43 | 0.008648963 |
| 200996_at | 43 | 0.008656316 |
| 236887_at | 43 | 0.008670965 |
| 204732_s_at | 43 | 0.008673403 |
| 204807_at | 43 | 0.008685198 |
| 205316_at | 43 | 0.008687064 |
| 236953_s_at | 43 | 0.008696678 |
| 223057_s_at | 43 | 0.008697954 |
| 1557852_at | 43 | 0.00870757 |
| 222538_s_at | 43 | 0.008708133 |
| 1558281_a_at | 43 | 0.008710351 |
| 200674_s_at | 43 | 0.008724484 |
| 239496_at | 43 | 0.008728771 |
| 224129_s_at | 43 | 0.008753507 |
| 214128_at | 43 | 0.008773002 |
| 235530_at | 43 | 0.00880806 |
| 204514_at | 43 | 0.008810146 |
| 233490_at | 43 | 0.008819732 |
| 215281_x_at | 43 | 0.00882645 |
| 1563497_at | 43 | 0.008827381 |
| 236404_at | 43 | 0.008828929 |
| 214697_s_at | 43 | 0.008830543 |
| 205645_at | 43 | 0.008834493 |
| 200664_s_at | 43 | 0.008843058 |
| 1555820_a_at | 43 | 0.008854502 |
| 1566003_x_at | 43 | 0.008870792 |
| 1558078_at | 43 | 0.008881897 |
| 223649_s_at | 43 | 0.00889929 |
| 222826_at | 43 | 0.008900678 |
| 1555568_at | 43 | 0.008906534 |
| 215385_at | 43 | 0.008913412 |
| 235811_at | 43 | 0.0089207 |
| 226024_at | 43 | 0.008928261 |
| 219957_at | 43 | 0.008931964 |
| 230795_at | 43 | 0.008934924 |
| 241790_at | 43 | 0.008938245 |
| 202145_at | 43 | 0.008945134 |
| 238666_at | 43 | 0.008946116 |
| 239383_at | 43 | 0.008971201 |
| 208227_x_at | 43 | 0.008971562 |
| 218236_s_at | 43 | 0.008979421 |
| 1568858_at | 43 | 0.008986275 |
| 226538_at | 43 | 0.009001207 |
| 242673_at | 43 | 0.009005341 |
| 1556014_at | 43 | 0.009017968 |
| 227292_at | 43 | 0.009022998 |
| 204803_s_at | 43 | 0.009025261 |
| 232061_at | 43 | 0.00903107 |
| 220762_s_at | 43 | 0.009032968 |
| 217968_at | 43 | 0.009035457 |
| 1560271_at | 43 | 0.009046833 |
| 222093_s_at | 43 | 0.009051329 |
| 222591_at | 43 | 0.009062086 |
| 229599_at | 43 | 0.009067384 |
| 219155_at | 43 | 0.009069477 |
| 223157_at | 43 | 0.009072978 |
| 215587_x_at | 43 | 0.009090049 |
| 242362_at | 43 | 0.00909253 |
| 223103_at | 43 | 0.009093796 |
| 221238_at | 43 | 0.009104637 |
| 223244_s_at | 43 | 0.009108425 |
| 232099_at | 43 | 0.009111139 |
| 220934_s_at | 43 | 0.009112428 |
| 204370_at | 43 | 0.009126536 |
| 221215_s_at | 43 | 0.009128139 |
| 233036_at | 43 | 0.009133727 |
| 243750_x_at | 43 | 0.009137252 |
| 220786_s_at | 43 | 0.009137394 |
| 218975_at | 43 | 0.009152355 |
| 1559141_s_at | 43 | 0.009157488 |
| 220658_s_at | 43 | 0.009160429 |
| 221745_at | 43 | 0.009160677 |
| 239585_at | 43 | 0.009169406 |
| 220260_at | 43 | 0.009174735 |
| 201577_at | 43 | 0.009177771 |
| 219945_at | 43 | 0.009184172 |
| 1557176_a_at | 43 | 0.009197983 |
| 235891_at | 43 | 0.009203676 |
| 241837_at | 43 | 0.009215945 |
| 212898_at | 43 | 0.00921977 |
| 1560128_x_at | 43 | 0.009222218 |
| 244845_at | 43 | 0.009224076 |
| 239086_at | 43 | 0.009229606 |
| 244801_at | 43 | 0.009236829 |
| 200757_s_at | 43 | 0.009237599 |
| 222372_at | 43 | 0.009253534 |
| 229871_at | 43 | 0.009271185 |
| 232192_at | 43 | 0.009271556 |
| 239241_at | 43 | 0.009286312 |
| 232064_at | 43 | 0.009288897 |
| 231198_at | 43 | 0.00929366 |
| 232637_at | 43 | 0.009294075 |
| 223586_at | 43 | 0.009298392 |
| 227291_s_at | 43 | 0.009301975 |
| 207769_s_at | 43 | 0.009302524 |
| 205405_at | 43 | 0.009312182 |
| 1555734_x_at | 43 | 0.009317318 |
| 222640_at | 43 | 0.00934223 |
| 217518_at | 43 | 0.009342341 |
| 232213_at | 43 | 0.009349652 |
| 218673_s_at | 43 | 0.00935227 |
| 205109_s_at | 43 | 0.00935746 |
| 204324_s_at | 43 | 0.009361976 |
| 226509_at | 43 | 0.009365981 |
| 1560137_at | 43 | 0.009366487 |
| 218967_s_at | 43 | 0.009371973 |
| 243830_at | 43 | 0.009374639 |
| 233289_at | 43 | 0.009379221 |
| 221629_x_at | 43 | 0.00939403 |
| 222944_s_at | 43 | 0.009394128 |
| 200762_at | 43 | 0.009405667 |
| 218596_at | 43 | 0.009410422 |
| 1557948_at | 43 | 0.009418548 |
| 1563781_at | 43 | 0.009419059 |
| 241954_at | 43 | 0.009422932 |
| 217965_s_at | 43 | 0.009434654 |
| 230681_at | 43 | 0.009435933 |
| 228523_at | 43 | 0.009446946 |
| 243293_at | 43 | 0.009457277 |
| 222896_at | 43 | 0.009461652 |
| 215156_at | 43 | 0.009462199 |
| 200869_at | 43 | 0.009494008 |
| 203028_s_at | 43 | 0.009502304 |
| 218094_s_at | 43 | 0.00950629 |
| 215212_at | 43 | 0.009506546 |
| 1053_at | 43 | 0.009509621 |
| 239285_at | 43 | 0.009517479 |
| 33814_at | 43 | 0.009541979 |
| 205516_x_at | 43 | 0.009542346 |
| 225241_at | 43 | 0.009554398 |
| 243178_at | 43 | 0.009558951 |
| 231741_at | 43 | 0.009570053 |
| 228141_at | 43 | 0.009576544 |
| 201936_s_at | 43 | 0.009578736 |
| 232453_at | 43 | 0.009581125 |
| 225742_at | 43 | 0.009590437 |
| 201774_s_at | 43 | 0.00959658 |
| 209175_at | 43 | 0.009628649 |
| 205806_at | 43 | 0.009633765 |
| 218281_at | 43 | 0.009638984 |
| 239694_at | 43 | 0.009640086 |
| 239283_at | 43 | 0.009643197 |
| 220467_at | 43 | 0.009657682 |
| 208343_s_at | 43 | 0.009657919 |
| 214427_at | 43 | 0.009663336 |
| 204540_at | 43 | 0.009675264 |
| 242235_x_at | 43 | 0.009677862 |
| 202603_at | 43 | 0.009679762 |
| 220255_at | 43 | 0.009682102 |
| 203696_s_at | 43 | 0.009700267 |
| 231985_at | 43 | 0.009706587 |
| 209965_s_at | 43 | 0.009724192 |
| 236704_at | 43 | 0.009736472 |
| 229112_at | 43 | 0.009736779 |
| 213166_x_at | 43 | 0.009743241 |
| 234578_at | 43 | 0.009749075 |
| 218278_at | 43 | 0.009753562 |
| 231854_at | 43 | 0.009766447 |
| 225378_at | 43 | 0.009777137 |
| 225947_at | 43 | 0.0097852 |
| 218353_at | 43 | 0.00979815 |
| 233632_s_at | 43 | 0.009815011 |
| 201391_at | 43 | 0.009826677 |
| 218385_at | 43 | 0.00982716 |
| 225053_at | 43 | 0.00982759 |
| 1557197_a_at | 43 | 0.009830365 |
| 224737_x_at | 43 | 0.00984299 |
| 226295_at | 43 | 0.009843188 |
| 239963_at | 43 | 0.00984801 |
| 219187_at | 43 | 0.009854181 |
| 212735_at | 43 | 0.009861965 |
| 204952_at | 43 | 0.009867477 |
| 205239_at | 43 | 0.009878081 |
| 209359_x_at | 43 | 0.009894407 |
| 206548_at | 43 | 0.009894746 |
| 226290_at | 43 | 0.009904834 |
| 239393_at | 43 | 0.009917622 |
| 213796_at | 43 | 0.009921602 |
| 228990_at | 43 | 0.009943502 |
| 218245_at | 43 | 0.009944825 |
| 65718_at | 43 | 0.009948445 |
| 213412_at | 43 | 0.00994965 |
| 212402_at | 43 | 0.009970354 |
| 211005_at | 43 | 0.009988499 |
| 233912_x_at | 43 | 0.009999351 |

TABLE 2
Probeset selected based on linear regression versus IC70 values

| Probeset (x value) | Count | PValue |
|---|---|---|
| 200894_s_at | 43 | 1.42E−06 |
| 201617_x_at | 43 | 1.5E−06 |
| 200895_s_at | 43 | 4.46E−06 |
| 228647_at | 43 | 7.25E−06 |
| 212450_at | 43 | 9.56E−06 |
| 218836_at | 43 | 1.51E−05 |
| 241627_x_at | 43 | 1.62E−05 |
| 215983_s_at | 43 | 2.17E−05 |
| 225504_at | 43 | 2.54E−05 |
| 201616_s_at | 43 | 3.25E−05 |
| 219429_at | 43 | 3.53E−05 |
| 224753_at | 43 | 3.55E−05 |
| 230118_at | 43 | 4.41E−05 |
| 212077_at | 43 | 5.72E−05 |
| 203867_s_at | 43 | 6.94E−05 |
| 220525_s_at | 43 | 7.08E−05 |
| 227685_at | 43 | 7.35E−05 |
| 208907_s_at | 43 | 9.03E−05 |
| 201307_at | 43 | 9.24E−05 |
| 217667_at | 43 | 9.52E−05 |
| 219050_s_at | 43 | 9.53E−05 |
| 204828_at | 43 | 9.68E−05 |
| 212778_at | 43 | 9.71E−05 |
| 219648_at | 43 | 0.000105 |
| 212239_at | 43 | 0.000125 |
| 213308_at | 43 | 0.000127 |
| 223641_at | 43 | 0.000135 |
| 209165_at | 43 | 0.000141 |
| 205613_at | 43 | 0.000144 |
| 226848_at | 43 | 0.000148 |
| 200809_x_at | 43 | 0.000149 |
| 211698_at | 43 | 0.000152 |
| 201342_at | 43 | 0.000158 |
| 224892_at | 43 | 0.000167 |
| 201312_s_at | 43 | 0.000183 |
| 221998_s_at | 43 | 0.000184 |
| 221729_at | 43 | 0.000185 |
| 209549_s_at | 43 | 0.000188 |
| 201483_s_at | 43 | 0.000188 |
| 1554063_at | 43 | 0.000191 |
| 225460_at | 43 | 0.000197 |
| 239253_at | 43 | 0.000199 |
| 221730_at | 43 | 0.0002 |
| 228189_at | 43 | 0.000202 |
| 230370_x_at | 43 | 0.000202 |
| 202594_at | 43 | 0.00021 |
| 1552330_at | 43 | 0.000215 |
| 201907_x_at | 43 | 0.000219 |
| 218050_at | 43 | 0.000223 |
| 201268_at | 43 | 0.000224 |
| 221256_s_at | 43 | 0.000231 |
| 224467_s_at | 43 | 0.000238 |
| 201615_x_at | 43 | 0.000251 |
| 228185_at | 43 | 0.00026 |
| 230606_at | 43 | 0.000262 |
| 224619_at | 43 | 0.000271 |
| 212116_at | 43 | 0.000277 |
| 235026_at | 43 | 0.00028 |
| 229205_at | 43 | 0.000292 |
| 209911_x_at | 43 | 0.000292 |
| 221820_s_at | 43 | 0.000294 |
| 204837_at | 43 | 0.000302 |
| 200095_x_at | 43 | 0.00031 |
| 201311_s_at | 43 | 0.000318 |
| 218147_s_at | 43 | 0.000318 |
| 227103_s_at | 43 | 0.000323 |
| 218321_x_at | 43 | 0.000323 |
| 200936_at | 43 | 0.000325 |
| 219071_x_at | 43 | 0.000327 |
| 229665_at | 43 | 0.000328 |
| 235114_x_at | 43 | 0.000331 |
| 230487_at | 43 | 0.000356 |
| 224875_at | 43 | 0.000357 |
| 202518_at | 43 | 0.000366 |
| 226007_at | 43 | 0.000368 |
| 1559776_at | 43 | 0.000373 |
| 214678_x_at | 43 | 0.000381 |
| 200658_s_at | 43 | 0.000383 |
| 209219_at | 43 | 0.000398 |
| 218567_x_at | 43 | 0.0004 |
| 211542_x_at | 43 | 0.000408 |
| 224479_s_at | 43 | 0.000413 |
| 201763_s_at | 43 | 0.000414 |
| 213077_at | 43 | 0.000417 |
| 227806_at | 43 | 0.000419 |
| 230702_at | 43 | 0.000454 |
| 207180_s_at | 43 | 0.00046 |
| 236165_at | 43 | 0.000465 |
| 218577_at | 43 | 0.000471 |
| 223728_at | 43 | 0.000495 |
| 34406_at | 43 | 0.000496 |
| 221606_s_at | 43 | 0.000504 |
| 203612_at | 43 | 0.000519 |
| 238002_at | 43 | 0.00052 |
| 228789_at | 43 | 0.000521 |
| 204808_s_at | 43 | 0.00053 |
| 233019_at | 43 | 0.000536 |
| 225223_at | 43 | 0.000543 |
| 233982_x_at | 43 | 0.000554 |
| 205428_s_at | 43 | 0.000554 |
| 224359_s_at | 43 | 0.00056 |
| 218437_s_at | 43 | 0.000597 |
| 227371_at | 43 | 0.000604 |
| 222551_s_at | 43 | 0.00061 |
| 224755_at | 43 | 0.000613 |
| 219148_at | 43 | 0.000615 |
| 209448_at | 43 | 0.000617 |
| 229289_at | 43 | 0.000622 |
| 221543_s_at | 43 | 0.000635 |
| 201901_s_at | 43 | 0.000644 |
| 204123_at | 43 | 0.000665 |
| 218405_at | 43 | 0.00067 |
| 232353_s_at | 43 | 0.000673 |
| 214880_x_at | 43 | 0.000674 |
| 218885_s_at | 43 | 0.000676 |
| 224876_at | 43 | 0.00068 |
| 204281_at | 43 | 0.000685 |
| 232510_s_at | 43 | 0.000686 |
| 226317_at | 43 | 0.000694 |
| 204630_s_at | 43 | 0.000709 |
| 200022_at | 43 | 0.000717 |
| 205525_at | 43 | 0.00073 |
| 225728_at | 43 | 0.00073 |
| 233263_at | 43 | 0.000731 |
| 221542_s_at | 43 | 0.000736 |
| 203816_at | 43 | 0.000752 |
| 235198_at | 43 | 0.000769 |
| 41037_at | 43 | 0.000777 |
| 225827_at | 43 | 0.000791 |
| 228587_at | 43 | 0.000796 |
| 226713_at | 43 | 0.000813 |
| 229287_at | 43 | 0.000814 |
| 221251_x_at | 43 | 0.000821 |
| 218136_s_at | 43 | 0.000834 |
| 203606_at | 43 | 0.000851 |
| 205998_x_at | 43 | 0.000851 |
| 235834_at | 43 | 0.000857 |
| 208009_s_at | 43 | 0.000859 |
| 219241_x_at | 43 | 0.000878 |
| 213099_at | 43 | 0.000887 |
| 211992_at | 43 | 0.000895 |
| 235786_at | 43 | 0.0009 |
| 214394_x_at | 43 | 0.0009 |
| 226661_at | 43 | 0.000908 |
| 214083_at | 43 | 0.000916 |
| 226785_at | 43 | 0.000918 |
| 225885_at | 43 | 0.00092 |
| 235817_at | 43 | 0.000924 |
| 200847_s_at | 43 | 0.000929 |
| 221081_s_at | 43 | 0.00093 |
| 242191_at | 43 | 0.000934 |
| 203911_at | 43 | 0.000934 |
| 213102_at | 43 | 0.000934 |
| 208669_s_at | 43 | 0.000937 |
| 227296_at | 43 | 0.00094 |
| 227221_at | 43 | 0.000941 |
| 238119_at | 43 | 0.000945 |
| 229053_at | 43 | 0.000949 |
| 210236_at | 43 | 0.000956 |
| 202494_at | 43 | 0.000957 |
| 238728_at | 43 | 0.000962 |
| 212630_at | 43 | 0.00097 |
| 90265_at | 43 | 0.000974 |
| 202777_at | 43 | 0.000978 |
| 242053_at | 43 | 0.000979 |
| 225217_s_at | 43 | 0.00098 |
| 229031_at | 43 | 0.000983 |
| 202380_s_at | 43 | 0.000993 |
| 201683_x_at | 43 | 0.000996 |
| 218860_at | 43 | 0.000998 |
| 218762_at | 43 | 0.001001 |
| 213155_at | 43 | 0.001021 |
| 226241_s_at | 43 | 0.001034 |
| 222846_at | 43 | 0.00104 |
| 228693_at | 43 | 0.001059 |
| 228603_at | 43 | 0.001066 |
| 222093_s_at | 43 | 0.001082 |
| 218803_at | 43 | 0.001086 |
| 232682_at | 43 | 0.001087 |
| 212229_s_at | 43 | 0.001093 |
| 213445_at | 43 | 0.001098 |
| 200088_x_at | 43 | 0.001134 |
| 210541_s_at | 43 | 0.001136 |
| 223574_x_at | 43 | 0.001142 |
| 200817_x_at | 43 | 0.001163 |
| 243259_at | 43 | 0.001165 |
| 239999_at | 43 | 0.001172 |
| 218187_s_at | 43 | 0.001177 |
| 211725_s_at | 43 | 0.001178 |
| 215545_at | 43 | 0.001178 |
| 224700_at | 43 | 0.001179 |
| 213227_at | 43 | 0.001188 |
| 219469_at | 43 | 0.001195 |
| 230127_at | 43 | 0.001196 |
| 227767_at | 43 | 0.001216 |
| 225743_at | 43 | 0.001221 |
| 220094_s_at | 43 | 0.001222 |
| 223261_at | 43 | 0.00123 |
| 212301_at | 43 | 0.001239 |
| 218146_at | 43 | 0.001242 |
| 214862_x_at | 43 | 0.001255 |
| 225948_at | 43 | 0.001258 |
| 200812_at | 43 | 0.001262 |
| 239476_at | 43 | 0.001264 |
| 228646_at | 43 | 0.001264 |
| 201934_at | 43 | 0.001267 |
| 213788_s_at | 43 | 0.001269 |
| 1562434_at | 43 | 0.001281 |
| 225837_at | 43 | 0.001287 |
| 213460_x_at | 43 | 0.001288 |
| 239238_at | 43 | 0.001293 |
| 243591_at | 43 | 0.001296 |
| 221069_s_at | 43 | 0.001297 |
| 203955_at | 43 | 0.001304 |
| 213174_at | 43 | 0.00132 |
| 207808_s_at | 43 | 0.001325 |
| 206506_s_at | 43 | 0.001338 |
| 235796_at | 43 | 0.00134 |
| 219350_s_at | 43 | 0.001364 |
| 202339_at | 43 | 0.001367 |
| 233588_x_at | 43 | 0.001368 |
| 214911_s_at | 43 | 0.001371 |
| 221712_s_at | 43 | 0.001371 |
| 206074_s_at | 43 | 0.001377 |
| 203884_s_at | 43 | 0.001387 |
| 207223_s_at | 43 | 0.00139 |
| 232527_at | 43 | 0.001392 |
| 201500_s_at | 43 | 0.001421 |
| 227456_s_at | 43 | 0.001428 |
| 209965_s_at | 43 | 0.001432 |
| 212700_x_at | 43 | 0.001444 |
| 224448_s_at | 43 | 0.001444 |
| 205333_s_at | 43 | 0.001447 |
| 1557238_s_at | 43 | 0.00145 |
| 201827_at | 43 | 0.001454 |
| 243088_at | 43 | 0.00146 |
| 219662_at | 43 | 0.001461 |
| 225384_at | 43 | 0.00147 |
| 205780_at | 43 | 0.00147 |
| 1558501_at | 43 | 0.001477 |
| 208685_x_at | 43 | 0.001481 |
| 202564_x_at | 43 | 0.001486 |
| 205103_at | 43 | 0.00149 |
| 226392_at | 43 | 0.001514 |
| 1556180_at | 43 | 0.001522 |
| 32541_at | 43 | 0.001525 |
| 225086_at | 43 | 0.001541 |
| 1555751_a_at | 43 | 0.001553 |
| 204076_at | 43 | 0.001569 |
| 242007_at | 43 | 0.001579 |
| 205768_s_at | 43 | 0.001582 |
| 221042_s_at | 43 | 0.001592 |
| 209428_s_at | 43 | 0.001594 |
| 221505_at | 43 | 0.001596 |
| 240038_at | 43 | 0.001598 |
| 205836_s_at | 43 | 0.001603 |
| 223056_s_at | 43 | 0.001606 |
| 47571_at | 43 | 0.001612 |
| 227033_at | 43 | 0.001646 |
| 218823_s_at | 43 | 0.001646 |
| 203458_at | 43 | 0.001649 |
| 227126_at | 43 | 0.001651 |
| 223649_s_at | 43 | 0.001657 |
| 226179_at | 43 | 0.00167 |
| 237065_s_at | 43 | 0.001671 |
| 203114_at | 43 | 0.001675 |
| 226601_at | 43 | 0.001689 |
| 219479_at | 43 | 0.0017 |
| 230991_at | 43 | 0.001702 |
| 202831_at | 43 | 0.001708 |
| 213070_at | 43 | 0.001708 |
| 217781_s_at | 43 | 0.001719 |
| 239519_at | 43 | 0.001725 |
| 242808_at | 43 | 0.001753 |
| 229073_at | 43 | 0.001767 |
| 210235_s_at | 43 | 0.001783 |
| 214527_s_at | 43 | 0.001816 |
| 226639_at | 43 | 0.001833 |
| 213695_at | 43 | 0.001842 |
| 241427_x_at | 43 | 0.001859 |
| 222998_at | 43 | 0.00186 |
| 228010_at | 43 | 0.001861 |
| 65133_i_at | 43 | 0.001865 |
| 227042_at | 43 | 0.001868 |
| 202029_x_at | 43 | 0.00187 |
| 1555878_at | 43 | 0.001872 |
| 212721_at | 43 | 0.001878 |
| 236192_at | 43 | 0.001886 |
| 214785_at | 43 | 0.001893 |
| 207143_at | 43 | 0.001896 |
| 212411_at | 43 | 0.001921 |
| 212997_s_at | 43 | 0.001926 |
| 231914_at | 43 | 0.001934 |
| 205037_at | 43 | 0.001967 |
| 225253_s_at | 43 | 0.001976 |
| 218978_s_at | 43 | 0.001979 |
| 204413_at | 43 | 0.001982 |
| 200686_s_at | 43 | 0.001995 |
| 203095_at | 43 | 0.001996 |
| 201684_s_at | 43 | 0.001997 |
| 200666_s_at | 43 | 0.002005 |
| 1558281_a_at | 43 | 0.002006 |
| 201045_s_at | 43 | 0.002026 |
| 225725_at | 43 | 0.002028 |
| 213131_at | 43 | 0.002039 |
| 226742_at | 43 | 0.002044 |
| 213278_at | 43 | 0.002049 |
| 212465_at | 43 | 0.002065 |
| 200785_s_at | 43 | 0.002083 |
| 228523_at | 43 | 0.002089 |
| 1557228_at | 43 | 0.002098 |
| 213085_s_at | 43 | 0.002105 |
| 214948_s_at | 43 | 0.002116 |
| 230071_at | 43 | 0.002126 |
| 225434_at | 43 | 0.002128 |
| 235792_x_at | 43 | 0.002128 |
| 233759_s_at | 43 | 0.002145 |
| 1556820_a_at | 43 | 0.002155 |
| 209717_at | 43 | 0.002161 |
| 233480_at | 43 | 0.002175 |
| 228803_at | 43 | 0.002185 |
| 201701_s_at | 43 | 0.002186 |
| 231727_s_at | 43 | 0.002193 |
| 206940_s_at | 43 | 0.002194 |
| 222475_at | 43 | 0.002207 |
| 208620_at | 43 | 0.002225 |
| 232522_at | 43 | 0.002232 |
| 222821_s_at | 43 | 0.002235 |
| 219531_at | 43 | 0.002253 |
| 243361_at | 43 | 0.002255 |
| 202827_s_at | 43 | 0.002258 |
| 1558401_at | 43 | 0.002266 |
| 228359_at | 43 | 0.002268 |
| 214814_at | 43 | 0.002268 |
| 205769_at | 43 | 0.002286 |
| 227191_at | 43 | 0.002304 |
| 209656_s_at | 43 | 0.002306 |
| 32402_s_at | 43 | 0.002309 |
| 225144_at | 43 | 0.002313 |
| 227561_at | 43 | 0.002319 |
| 203927_at | 43 | 0.002323 |
| 223060_at | 43 | 0.002333 |
| 227693_at | 43 | 0.002334 |
| 213773_x_at | 43 | 0.002334 |
| 235437_at | 43 | 0.002349 |
| 213145_at | 43 | 0.002349 |
| 233101_at | 43 | 0.002352 |
| 1555943_at | 43 | 0.002361 |
| 232322_x_at | 43 | 0.002367 |
| 230905_at | 43 | 0.002375 |
| 203131_at | 43 | 0.002388 |
| 219420_s_at | 43 | 0.0024 |
| 213414_s_at | 43 | 0.002417 |
| 218196_at | 43 | 0.002426 |
| 202080_s_at | 43 | 0.002434 |
| 203608_at | 43 | 0.002438 |
| 223580_at | 43 | 0.002458 |
| 227628_at | 43 | 0.002464 |
| 227455_at | 43 | 0.002467 |
| 213122_at | 43 | 0.002469 |
| 227639_at | 43 | 0.002472 |
| 231106_at | 43 | 0.002479 |
| 227536_at | 43 | 0.002484 |
| 235220_at | 43 | 0.002487 |
| 207396_s_at | 43 | 0.002496 |
| 233274_at | 43 | 0.002511 |
| 231852_at | 43 | 0.002521 |
| 226240_at | 43 | 0.002532 |
| 224743_at | 43 | 0.002544 |
| 214271_x_at | 43 | 0.002548 |
| 231146_at | 43 | 0.002565 |
| 208600_s_at | 43 | 0.00258 |
| 238504_at | 43 | 0.002581 |
| 229235_at | 43 | 0.002583 |
| 201560_at | 43 | 0.002595 |
| 1565804_at | 43 | 0.002596 |
| 216995_x_at | 43 | 0.002603 |
| 202923_s_at | 43 | 0.002604 |
| 235566_at | 43 | 0.002616 |
| 1560926_at | 43 | 0.002623 |
| 212202_s_at | 43 | 0.002625 |
| 201639_s_at | 43 | 0.002625 |
| 225074_at | 43 | 0.002634 |
| 224737_x_at | 43 | 0.002648 |
| 200997_at | 43 | 0.00265 |
| 222773_s_at | 43 | 0.002656 |
| 229535_at | 43 | 0.002656 |
| 203156_at | 43 | 0.002664 |
| 203866_at | 43 | 0.002677 |
| 212440_at | 43 | 0.002688 |
| 219187_at | 43 | 0.00269 |
| 209115_at | 43 | 0.002693 |
| 201613_s_at | 43 | 0.002698 |
| 225879_at | 43 | 0.002699 |
| 224883_at | 43 | 0.002701 |
| 225229_at | 43 | 0.002706 |
| 225334_at | 43 | 0.002711 |
| 213527_s_at | 43 | 0.002713 |
| 203253_s_at | 43 | 0.002722 |
| 205224_at | 43 | 0.002727 |
| 226395_at | 43 | 0.002734 |
| 217969_at | 43 | 0.00274 |
| 223619_x_at | 43 | 0.002748 |
| 1555938_x_at | 43 | 0.002755 |
| 213703_at | 43 | 0.002763 |
| 240008_at | 43 | 0.002773 |
| 241970_at | 43 | 0.002775 |
| 242208_at | 43 | 0.002788 |
| 204441_s_at | 43 | 0.002792 |
| 244433_at | 43 | 0.002796 |
| 200800_s_at | 43 | 0.002802 |
| 212626_x_at | 43 | 0.002807 |
| 1569114_at | 43 | 0.002808 |
| 213189_at | 43 | 0.002816 |
| 214046_at | 43 | 0.002818 |
| 212900_at | 43 | 0.002821 |
| 218871_x_at | 43 | 0.002826 |
| 219628_at | 43 | 0.00283 |
| 223245_at | 43 | 0.002833 |
| 213166_x_at | 43 | 0.002834 |
| 235585_at | 43 | 0.00284 |
| 229666_s_at | 43 | 0.002858 |
| 235071_at | 43 | 0.002861 |
| 238299_at | 43 | 0.002873 |
| 241906_at | 43 | 0.002876 |
| 225442_at | 43 | 0.002889 |
| 223931_s_at | 43 | 0.002901 |
| 240991_at | 43 | 0.002902 |
| 1559332_at | 43 | 0.002908 |
| 225395_s_at | 43 | 0.002932 |
| 1553193_at | 43 | 0.002932 |
| 236619_at | 43 | 0.002932 |
| 228462_at | 43 | 0.002952 |
| 202034_x_at | 43 | 0.002958 |
| 202603_at | 43 | 0.002968 |
| 204465_s_at | 43 | 0.002969 |
| 222884_at | 43 | 0.002971 |
| 233364_s_at | 43 | 0.002993 |
| 201687_s_at | 43 | 0.002999 |
| 209884_s_at | 43 | 0.00302 |
| 235103_at | 43 | 0.003034 |
| 214132_at | 43 | 0.003036 |
| 229022_at | 43 | 0.00304 |
| 200051_at | 43 | 0.003045 |
| 201346_at | 43 | 0.003056 |
| 217910_x_at | 43 | 0.003064 |
| 215412_x_at | 43 | 0.003079 |
| 219906_at | 43 | 0.00308 |
| 225878_at | 43 | 0.003083 |
| 201533_at | 43 | 0.003085 |
| 229789_at | 43 | 0.003095 |
| 223808_s_at | 43 | 0.003095 |
| 231854_at | 43 | 0.003103 |
| 228479_at | 43 | 0.003111 |
| 219919_s_at | 43 | 0.00312 |
| 230958_s_at | 43 | 0.003134 |
| 206128_at | 43 | 0.003142 |
| 202645_s_at | 43 | 0.003143 |
| 222029_x_at | 43 | 0.003143 |
| 226338_at | 43 | 0.003151 |
| 212515_s_at | 43 | 0.003153 |
| 206683_at | 43 | 0.003153 |
| 235280_at | 43 | 0.003162 |
| 239258_at | 43 | 0.003172 |
| 227568_at | 43 | 0.003183 |
| 235031_at | 43 | 0.003186 |
| 218732_at | 43 | 0.003186 |
| 209832_s_at | 43 | 0.00319 |
| 201852_x_at | 43 | 0.00319 |
| 1556818_at | 43 | 0.003195 |
| 226502_at | 43 | 0.003203 |
| 223305_at | 43 | 0.003208 |
| 214073_at | 43 | 0.003235 |
| 236967_at | 43 | 0.003253 |
| 1568815_a_at | 43 | 0.003254 |
| 202178_at | 43 | 0.003255 |
| 221935_s_at | 43 | 0.003261 |
| 201484_at | 43 | 0.003265 |
| 205881_at | 43 | 0.003274 |
| 226921_at | 43 | 0.003276 |
| 219526_at | 43 | 0.003279 |
| 212887_at | 43 | 0.003321 |
| 222792_s_at | 43 | 0.003325 |
| 217786_at | 43 | 0.003326 |
| 1563467_at | 43 | 0.003329 |
| 222267_at | 43 | 0.003333 |
| 200689_x_at | 43 | 0.003341 |
| 218708_at | 43 | 0.003346 |
| 229692_at | 43 | 0.00335 |
| 238037_at | 43 | 0.003353 |
| 232057_at | 43 | 0.003353 |
| 227804_at | 43 | 0.003365 |
| 219168_s_at | 43 | 0.003379 |
| 33132_at | 43 | 0.003383 |
| 228407_at | 43 | 0.003384 |
| 238606_at | 43 | 0.003399 |
| 1558802_at | 43 | 0.003403 |
| 208758_at | 43 | 0.003417 |
| 227143_s_at | 43 | 0.003423 |
| 209008_x_at | 43 | 0.003429 |
| 216439_at | 43 | 0.003443 |
| 226633_at | 43 | 0.003445 |
| 200046_at | 43 | 0.003448 |
| 224744_at | 43 | 0.003482 |
| 213893_x_at | 43 | 0.003494 |
| 225901_at | 43 | 0.003505 |
| 51192_at | 43 | 0.003506 |
| 221754_s_at | 43 | 0.003517 |
| 226416_at | 43 | 0.003518 |
| 224704_at | 43 | 0.00354 |
| 217938_s_at | 43 | 0.003541 |
| 223103_at | 43 | 0.003551 |
| 229773_at | 43 | 0.003577 |
| 213175_s_at | 43 | 0.003586 |
| 203113_s_at | 43 | 0.003586 |
| 238894_at | 43 | 0.003589 |
| 1569167_at | 43 | 0.003624 |
| 224462_s_at | 43 | 0.003633 |
| 223411_at | 43 | 0.003635 |
| 212110_at | 43 | 0.003651 |
| 228090_at | 43 | 0.003653 |
| 238524_at | 43 | 0.003654 |
| 219245_s_at | 43 | 0.003655 |
| 201440_at | 43 | 0.003657 |
| 211345_x_at | 43 | 0.003664 |
| 218630_at | 43 | 0.003669 |
| 225523_at | 43 | 0.003677 |
| 50374_at | 43 | 0.003681 |
| 218069_at | 43 | 0.003688 |
| 212114_at | 43 | 0.003692 |
| 236080_at | 43 | 0.003729 |
| 48106_at | 43 | 0.003739 |
| 203802_x_at | 43 | 0.003745 |
| 232455_x_at | 43 | 0.003762 |
| 218841_at | 43 | 0.003768 |
| 200932_s_at | 43 | 0.003771 |
| 223293_at | 43 | 0.003818 |
| 229431_at | 43 | 0.003818 |
| 209537_at | 43 | 0.003827 |
| 218348_s_at | 43 | 0.003836 |
| 214100_x_at | 43 | 0.00384 |
| 205759_s_at | 43 | 0.003847 |
| 216449_x_at | 43 | 0.003862 |
| 229901_at | 43 | 0.003866 |
| 201161_s_at | 43 | 0.003868 |
| 228946_at | 43 | 0.003876 |
| 213157_s_at | 43 | 0.00388 |
| 1559023_a_at | 43 | 0.003885 |
| 205093_at | 43 | 0.00389 |
| 39817_s_at | 43 | 0.003912 |
| 244414_at | 43 | 0.003918 |
| 213529_at | 43 | 0.003922 |
| 217713_x_at | 43 | 0.00393 |
| 1554474_a_at | 43 | 0.003934 |
| 207046_at | 43 | 0.003938 |
| 226873_at | 43 | 0.003942 |
| 235373_at | 43 | 0.003943 |
| 209516_at | 43 | 0.003966 |
| 1570338_at | 43 | 0.003986 |
| 242669_at | 43 | 0.003995 |
| 225318_at | 43 | 0.004001 |
| 204936_at | 43 | 0.004006 |
| 225947_at | 43 | 0.004008 |
| 210059_s_at | 43 | 0.004024 |
| 1554678_s_at | 43 | 0.004025 |
| 214372_x_at | 43 | 0.004031 |
| 232613_at | 43 | 0.004057 |
| 226565_at | 43 | 0.004057 |
| 212163_at | 43 | 0.004062 |
| 205854_at | 43 | 0.004066 |
| 232371_at | 43 | 0.004068 |
| 235318_at | 43 | 0.004075 |
| 224695_at | 43 | 0.004078 |
| 236375_at | 43 | 0.004078 |
| 244659_at | 43 | 0.00408 |
| 235646_at | 43 | 0.004086 |
| 225219_at | 43 | 0.004087 |
| 225696_at | 43 | 0.004087 |
| 227687_at | 43 | 0.004112 |
| 212563_at | 43 | 0.004113 |
| 243253_at | 43 | 0.004139 |
| 205704_s_at | 43 | 0.004141 |
| 209467_s_at | 43 | 0.004153 |
| 215631_s_at | 43 | 0.004159 |
| 242558_at | 43 | 0.004165 |
| 208848_at | 43 | 0.004167 |
| 217939_s_at | 43 | 0.004167 |
| 218145_at | 43 | 0.004169 |
| 212172_at | 43 | 0.004177 |
| 209431_s_at | 43 | 0.004181 |
| 218596_at | 43 | 0.004193 |
| 22653 8_at | 43 | 0.004193 |
| 209341_s_at | 43 | 0.004198 |
| 235396_at | 43 | 0.004212 |
| 212981_s_at | 43 | 0.004217 |
| 218816_at | 43 | 0.004227 |
| 221920_s_at | 43 | 0.004231 |
| 210627_s_at | 43 | 0.004235 |
| 215855_s_at | 43 | 0.004251 |
| 227579_at | 43 | 0.004264 |
| 203755_at | 43 | 0.004264 |
| 226146_at | 43 | 0.004269 |
| 222111_at | 43 | 0.004281 |
| 1559352_a_at | 43 | 0.004292 |
| 219988_s_at | 43 | 0.004302 |
| 203004_s_at | 43 | 0.004305 |
| 213708_s_at | 43 | 0.004307 |
| 210848_at | 43 | 0.004358 |
| 240815_at | 43 | 0.004358 |
| 226129_at | 43 | 0.004361 |
| 244610_x_at | 43 | 0.004374 |
| 237561_x_at | 43 | 0.004387 |
| 1555960_at | 43 | 0.004393 |
| 218481_at | 43 | 0.004402 |
| 236454_at | 43 | 0.004416 |
| 230388_s_at | 43 | 0.004424 |
| 223157_at | 43 | 0.004432 |
| 215383_x_at | 43 | 0.00444 |
| 226584_s_at | 43 | 0.004442 |
| 221797_at | 43 | 0.00445 |
| 233678_at | 43 | 0.004467 |
| 211161_s_at | 43 | 0.004469 |
| 218914_at | 43 | 0.00447 |
| 223382_s_at | 43 | 0.004478 |
| 230177_at | 43 | 0.004488 |
| 239694_at | 43 | 0.004489 |
| 213621_s_at | 43 | 0.004493 |
| 222703_s_at | 43 | 0.004493 |
| 227262_at | 43 | 0.004494 |
| 201031_s_at | 43 | 0.004507 |
| 227102_at | 43 | 0.004511 |
| 213520_at | 43 | 0.004528 |
| 203261_at | 43 | 0.004536 |
| 218125_s_at | 43 | 0.004539 |
| 201409_s_at | 43 | 0.004541 |
| 240261_at | 43 | 0.004566 |
| 239571_at | 43 | 0.004577 |
| 202766_s_at | 43 | 0.004577 |
| 204514_at | 43 | 0.004584 |
| 232235_at | 43 | 0.004591 |
| 218316_at | 43 | 0.004596 |
| 214731_at | 43 | 0.004601 |
| 226509_at | 43 | 0.004602 |
| 212872_s_at | 43 | 0.004609 |
| 214093_s_at | 43 | 0.004623 |
| 201407_s_at | 43 | 0.004623 |
| 202649_x_at | 43 | 0.004624 |
| 223815_at | 43 | 0.004625 |
| 229758_at | 43 | 0.004635 |
| 227445_at | 43 | 0.004639 |
| 1569999_at | 43 | 0.004641 |
| 223238_s_at | 43 | 0.004645 |
| 207605_x_at | 43 | 0.004649 |
| 217810_x_at | 43 | 0.004657 |
| 217408_at | 43 | 0.004673 |
| 220468_at | 43 | 0.004685 |
| 219203_at | 43 | 0.004696 |
| 220934_s_at | 43 | 0.004704 |
| 228141_at | 43 | 0.004707 |
| 222746_s_at | 43 | 0.00471 |
| 235756_at | 43 | 0.004714 |
| 218804_at | 43 | 0.004717 |
| 205205_at | 43 | 0.004721 |
| 212351_at | 43 | 0.00474 |
| 214241_at | 43 | 0.00475 |
| 205516_x_at | 43 | 0.004757 |
| 1558329_at | 43 | 0.004778 |
| 216074_x_at | 43 | 0.004796 |
| 206357_at | 43 | 0.004804 |
| 223048_at | 43 | 0.004823 |
| 225950_at | 43 | 0.004826 |
| 215075_s_at | 43 | 0.00483 |
| 215980_s_at | 43 | 0.004849 |
| 227292_at | 43 | 0.004851 |
| 213670_x_at | 43 | 0.004862 |
| 204868_at | 43 | 0.004862 |
| 238735_at | 43 | 0.004866 |
| 205168_at | 43 | 0.004868 |
| 203286_at | 43 | 0.004913 |
| 208109_s_at | 43 | 0.004935 |
| 201400_at | 43 | 0.004951 |
| 222344_at | 43 | 0.004953 |
| 227940_at | 43 | 0.004977 |
| 221215_s_at | 43 | 0.005008 |
| 213913_s_at | 43 | 0.005009 |
| 242871_at | 43 | 0.005031 |
| 222376_at | 43 | 0.005031 |
| 1557737_s_at | 43 | 0.005046 |
| 242422_at | 43 | 0.005052 |
| 239432_at | 43 | 0.005061 |
| 1555820_a_at | 43 | 0.005067 |
| 200599_s_at | 43 | 0.00507 |
| 203791_at | 43 | 0.00507 |
| 213089_at | 43 | 0.005081 |
| 224331_s_at | 43 | 0.005086 |
| 225476_at | 43 | 0.005097 |
| 224336_s_at | 43 | 0.005099 |
| 232067_at | 43 | 0.005102 |
| 230443_at | 43 | 0.005125 |
| 213402_at | 43 | 0.005136 |
| 225406_at | 43 | 0.00515 |
| 235545_at | 43 | 0.005153 |
| 219162_s_at | 43 | 0.00516 |
| 227008_at | 43 | 0.005161 |
| 223156_at | 43 | 0.005178 |
| 242222_at | 43 | 0.005179 |
| 209739_s_at | 43 | 0.005181 |
| 221836_s_at | 43 | 0.005197 |
| 207769_s_at | 43 | 0.00521 |
| 208612_at | 43 | 0.005219 |
| 203433_at | 43 | 0.005235 |
| 218670_at | 43 | 0.005237 |
| 226499_at | 43 | 0.005267 |
| 213842_x_at | 43 | 0.00528 |
| 200036_s_at | 43 | 0.005304 |
| 226286_at | 43 | 0.005304 |
| 1556126_s_at | 43 | 0.005304 |
| 219411_at | 43 | 0.005307 |
| 225475_at | 43 | 0.005308 |
| 202176_at | 43 | 0.005331 |
| 207829_s_at | 43 | 0.005332 |
| 235197_s_at | 43 | 0.005334 |
| 202396_at | 43 | 0.005355 |
| 236154_at | 43 | 0.005356 |
| 222372_s_at | 43 | 0.005359 |
| 212685_s_at | 43 | 0.005367 |
| 1559154_at | 43 | 0.005375 |
| 226318_at | 43 | 0.005378 |
| 205667_at | 43 | 0.005385 |
| 1556151_at | 43 | 0.005399 |
| 235871_at | 43 | 0.0054 |
| 207000_s_at | 43 | 0.00541 |
| 222310_at | 43 | 0.005419 |
| 200651_at | 43 | 0.005426 |
| 1552286_at | 43 | 0.00543 |
| 228338_at | 43 | 0.005431 |
| 235359_at | 43 | 0.005432 |
| 222732_at | 43 | 0.005436 |
| 215560_x_at | 43 | 0.005442 |
| 209893_s_at | 43 | 0.005456 |
| 38710_at | 43 | 0.005473 |
| 244026_at | 43 | 0.005496 |
| 201391_at | 43 | 0.005499 |
| 205991_s_at | 43 | 0.005521 |
| 1568987_at | 43 | 0.005533 |
| 217912_at | 43 | 0.005555 |
| 236769_at | 43 | 0.005563 |
| 1552364_s_at | 43 | 0.005564 |
| 224903_at | 43 | 0.005573 |
| 232175_at | 43 | 0.005584 |
| 228980_at | 43 | 0.005596 |
| 232865_at | 43 | 0.005602 |
| 203342_at | 43 | 0.005607 |
| 243681_at | 43 | 0.00561 |
| 200019_s_at | 43 | 0.005614 |
| 218236_s_at | 43 | 0.005636 |
| 210502_s_at | 43 | 0.005639 |
| 209430_at | 43 | 0.005664 |
| 208670_s_at | 43 | 0.005666 |
| 242283_at | 43 | 0.005689 |
| 219378_at | 43 | 0.005692 |
| 213111_at | 43 | 0.005705 |
| 214949_at | 43 | 0.005706 |
| 219150_s_at | 43 | 0.005717 |
| 221973_at | 43 | 0.005739 |
| 1557242_at | 43 | 0.005747 |
| 218245_at | 43 | 0.005753 |
| 215076_s_at | 43 | 0.005763 |
| 211936_at | 43 | 0.005767 |
| 243046_at | 43 | 0.005773 |
| 224129_s_at | 43 | 0.005781 |
| 212400_at | 43 | 0.005784 |
| 221478_at | 43 | 0.005789 |
| 202375_at | 43 | 0.005799 |
| 238146_at | 43 | 0.005802 |
| 227595_at | 43 | 0.00582 |
| 233173_x_at | 43 | 0.005823 |
| 236531_at | 43 | 0.005832 |
| 211214_s_at | 43 | 0.005836 |
| 213812_s_at | 43 | 0.005844 |
| 212847_at | 43 | 0.005851 |
| 244791_at | 43 | 0.005855 |
| 230035_at | 43 | 0.005869 |
| 236367_at | 43 | 0.005886 |
| 232975_at | 43 | 0.005895 |
| 212544_at | 43 | 0.005902 |
| 202394_s_at | 43 | 0.005904 |
| 230361_at | 43 | 0.005908 |
| 241907_at | 43 | 0.005919 |
| 209655_s_at | 43 | 0.005921 |
| 220762_s_at | 43 | 0.005924 |
| 235647_at | 43 | 0.005924 |
| 209873_s_at | 43 | 0.005966 |
| 201459_at | 43 | 0.00598 |
| 220160_s_at | 43 | 0.005983 |
| 220355_s_at | 43 | 0.005985 |
| 201719_s_at | 43 | 0.005986 |
| 224656_s_at | 43 | 0.005987 |
| 224966_s_at | 43 | 0.005992 |
| 222141_at | 43 | 0.006056 |
| 243801_x_at | 43 | 0.006061 |
| 201584_s_at | 43 | 0.006069 |
| 241630_at | 43 | 0.00608 |
| 242514_at | 43 | 0.006082 |
| 204540_at | 43 | 0.006092 |
| 1556657_at | 43 | 0.006095 |
| 238342_at | 43 | 0.0061 |
| 200659_s_at | 43 | 0.006115 |
| 226619_at | 43 | 0.006115 |
| 229218_at | 43 | 0.006119 |
| 236649_at | 43 | 0.006137 |
| 1564796_at | 43 | 0.00614 |
| 222826_at | 43 | 0.006152 |
| 224565_at | 43 | 0.006154 |
| 210946_at | 43 | 0.006169 |
| 212115_at | 43 | 0.00617 |
| 218165_at | 43 | 0.006182 |
| 1555068_at | 43 | 0.006182 |
| 238550_at | 43 | 0.006188 |
| 203274_at | 43 | 0.006194 |
| 235063_at | 43 | 0.0062 |
| 241808_at | 43 | 0.006209 |
| 225185_at | 43 | 0.006218 |
| 212877_at | 43 | 0.00624 |
| 204148_s_at | 43 | 0.006246 |
| 209517_s_at | 43 | 0.006246 |
| 224691_at | 43 | 0.006247 |
| 222235_s_at | 43 | 0.006259 |
| 210213_s_at | 43 | 0.006274 |
| 228024_at | 43 | 0.00628 |
| 1568853_at | 43 | 0.006286 |
| 225975_at | 43 | 0.006287 |
| 233440_at | 43 | 0.006288 |
| 235890_at | 43 | 0.00632 |
| 213125_at | 43 | 0.006331 |
| 226831_at | 43 | 0.006335 |
| 211937_at | 43 | 0.006347 |
| 235089_at | 43 | 0.006356 |
| 221732_at | 43 | 0.006362 |
| 203244_at | 43 | 0.006373 |
| 214756_x_at | 43 | 0.006374 |
| 203525_s_at | 43 | 0.006381 |
| 200816_s_at | 43 | 0.006384 |
| 222470_s_at | 43 | 0.006405 |
| 218928_s_at | 43 | 0.00641 |
| 218016_s_at | 43 | 0.006427 |
| 218214_at | 43 | 0.006443 |
| 204493_at | 43 | 0.006453 |
| 230452_at | 43 | 0.006456 |
| 1554411_at | 43 | 0.006464 |
| 200007_at | 43 | 0.006479 |
| 226105_at | 43 | 0.006484 |
| 204565_at | 43 | 0.0065 |
| 205538_at | 43 | 0.006516 |
| 227761_at | 43 | 0.006519 |
| 244441_at | 43 | 0.006536 |
| 217610_at | 43 | 0.006549 |
| 214937_x_at | 43 | 0.006573 |
| 217935_s_at | 43 | 0.006578 |
| 209161_at | 43 | 0.006583 |
| 214293_at | 43 | 0.006585 |
| 226217_at | 43 | 0.006588 |
| 210449_x_at | 43 | 0.006588 |
| 221834_at | 43 | 0.00659 |
| 217925_s_at | 43 | 0.006593 |
| 223400_s_at | 43 | 0.006598 |
| 210123_s_at | 43 | 0.006606 |
| 210242_x_at | 43 | 0.006608 |
| 236288_at | 43 | 0.006611 |
| 211927_x_at | 43 | 0.006629 |
| 203024_s_at | 43 | 0.006637 |
| 202580_x_at | 43 | 0.006645 |
| 212467_at | 43 | 0.006649 |
| 210822_at | 43 | 0.006662 |
| 224882_at | 43 | 0.006662 |
| 203150_at | 43 | 0.006668 |
| 203573_s_at | 43 | 0.006698 |
| 200877_at | 43 | 0.006711 |
| 233870_at | 43 | 0.006721 |
| 219293_s_at | 43 | 0.006722 |
| 234074_at | 43 | 0.006725 |
| 228159_at | 43 | 0.006743 |
| 210205_at | 43 | 0.006758 |
| 201046_s_at | 43 | 0.006783 |
| 202145_at | 43 | 0.006795 |
| 239672_at | 43 | 0.006795 |
| 235158_at | 43 | 0.006799 |
| 209175_at | 43 | 0.006808 |
| 232641_at | 43 | 0.006813 |
| 200996_at | 43 | 0.006819 |
| 241965_at | 43 | 0.006837 |
| 1552803_a_at | 43 | 0.006842 |
| 200863_s_at | 43 | 0.006845 |
| 202174_s_at | 43 | 0.006869 |
| 218907_s_at | 43 | 0.006893 |
| 232218_at | 43 | 0.006898 |
| 1563455_at | 43 | 0.006933 |
| 226938_at | 43 | 0.006942 |
| 200755_s_at | 43 | 0.006951 |
| 226515_at | 43 | 0.00697 |
| 218383_at | 43 | 0.006974 |
| 1558620_at | 43 | 0.006978 |
| 203628_at | 43 | 0.006986 |
| 243673_at | 43 | 0.00699 |
| 201393_s_at | 43 | 0.006998 |
| 219406_at | 43 | 0.007028 |
| 203688_at | 43 | 0.007028 |
| 238712_at | 43 | 0.007033 |
| 210908_s_at | 43 | 0.007039 |
| 217448_s_at | 43 | 0.007044 |
| 213684_s_at | 43 | 0.007048 |
| 205591_at | 43 | 0.007048 |
| 224248_x_at | 43 | 0.007049 |
| 226935_s_at | 43 | 0.007053 |
| 236072_at | 43 | 0.00706 |
| 225653_at | 43 | 0.007062 |
| 236435_at | 43 | 0.007078 |
| 225984_at | 43 | 0.007087 |
| 219455_at | 43 | 0.007099 |
| 216147_at | 43 | 0.007102 |
| 210652_s_at | 43 | 0.007104 |
| 202326_at | 43 | 0.007136 |
| 224865_at | 43 | 0.007145 |
| 229443_at | 43 | 0.007151 |
| 202264_s_at | 43 | 0.007164 |
| 212986_s_at | 43 | 0.007171 |
| 238852_at | 43 | 0.007172 |
| 212113_at | 43 | 0.007174 |
| 201395_at | 43 | 0.007175 |
| 223406_x_at | 43 | 0.007176 |
| 225365_at | 43 | 0.007177 |
| 230656_s_at | 43 | 0.007186 |
| 205771_s_at | 43 | 0.007192 |
| 200935_at | 43 | 0.007206 |
| 213880_at | 43 | 0.007207 |
| 229590_at | 43 | 0.007209 |
| 1564238_a_at | 43 | 0.007234 |
| 224352_s_at | 43 | 0.007253 |
| 1558116_x_at | 43 | 0.007266 |
| 241838_at | 43 | 0.007274 |
| 209308_s_at | 43 | 0.007297 |
| 208503_s_at | 43 | 0.0073 |
| 218385_at | 43 | 0.007332 |
| 204403_x_at | 43 | 0.007343 |
| 209162_s_at | 43 | 0.007348 |
| 1560579_s_at | 43 | 0.007351 |
| 218897_at | 43 | 0.007365 |
| 203883_s_at | 43 | 0.007383 |
| 204238_s_at | 43 | 0.007401 |
| 235530_at | 43 | 0.007405 |
| 222814_s_at | 43 | 0.007413 |
| 202404_s_at | 43 | 0.007414 |
| 211341_at | 43 | 0.007422 |
| 219548_at | 43 | 0.007436 |
| 201913_s_at | 43 | 0.007438 |
| 232113_at | 43 | 0.007441 |
| 201256_at | 43 | 0.007442 |
| 204572_s_at | 43 | 0.007444 |
| 204366_s_at | 43 | 0.007456 |
| 220926_s_at | 43 | 0.007458 |
| 219201_s_at | 43 | 0.007458 |
| 1556769_a_at | 43 | 0.007466 |
| 218674_at | 43 | 0.007472 |
| 37226_at | 43 | 0.007473 |
| 205621_at | 43 | 0.007475 |
| 212982_at | 43 | 0.007484 |
| 202351_at | 43 | 0.007491 |
| 36865_at | 43 | 0.007504 |
| 228009_x_at | 43 | 0.00751 |
| 231437_at | 43 | 0.00754 |
| 228320_x_at | 43 | 0.007544 |
| 218278_at | 43 | 0.007565 |
| 229215_at | 43 | 0.007579 |
| 226531_at | 43 | 0.007587 |
| 225044_at | 43 | 0.007597 |
| 202811_at | 43 | 0.0076 |
| 203644_s_at | 43 | 0.007616 |
| 204091_at | 43 | 0.007634 |
| 227395_at | 43 | 0.007644 |
| 223020_at | 43 | 0.007662 |
| 1564520_s_at | 43 | 0.007665 |
| 217975_at | 43 | 0.00767 |
| 216524_x_at | 43 | 0.007672 |
| 207971_s_at | 43 | 0.007673 |
| 240098_at | 43 | 0.007674 |
| 234343_s_at | 43 | 0.007681 |
| 217279_x_at | 43 | 0.007695 |
| 221899_at | 43 | 0.007704 |
| 217598_at | 43 | 0.007721 |
| 225841_at | 43 | 0.007721 |
| 232459_at | 43 | 0.00773 |
| 200846_s_at | 43 | 0.007734 |
| 210792_x_at | 43 | 0.007735 |
| 35148_at | 43 | 0.007744 |
| 226293_at | 43 | 0.00775 |
| 222236_s_at | 43 | 0.007756 |
| 243750_x_at | 43 | 0.007757 |
| 1559946_s_at | 43 | 0.007787 |
| 225301_s_at | 43 | 0.007788 |
| 228558_at | 43 | 0.007806 |
| 228432_at | 43 | 0.007809 |
| 218285_s_at | 43 | 0.007809 |
| 201580_s_at | 43 | 0.007814 |
| 208723_at | 43 | 0.007829 |
| 236201_at | 43 | 0.007864 |
| 229483_at | 43 | 0.007866 |
| 232403_at | 43 | 0.007872 |
| 230728_at | 43 | 0.007875 |
| 212925_at | 43 | 0.007877 |
| 241993_x_at | 43 | 0.007879 |
| 242619_x_at | 43 | 0.007885 |
| 205407_at | 43 | 0.007892 |
| 205775_at | 43 | 0.007895 |
| 242688_at | 43 | 0.007904 |
| 218267_at | 43 | 0.007914 |
| 214845_s_at | 43 | 0.007917 |
| 228570_at | 43 | 0.007923 |
| 203799_at | 43 | 0.007931 |
| 212454_x_at | 43 | 0.007961 |
| 201353_s_at | 43 | 0.007962 |
| 223062_s_at | 43 | 0.007978 |
| 232058_at | 43 | 0.007987 |
| 229573_at | 43 | 0.007993 |
| 206921_at | 43 | 0.007997 |
| 226121_at | 43 | 0.008054 |
| 235588_at | 43 | 0.008071 |
| 223631_s_at | 43 | 0.008072 |
| 229549_at | 43 | 0.008092 |
| 213172_at | 43 | 0.008099 |
| 203407_at | 43 | 0.008099 |
| 224671_at | 43 | 0.008119 |
| 239350_at | 43 | 0.008121 |
| 207856_s_at | 43 | 0.008123 |
| 223825_at | 43 | 0.008145 |
| 221704_s_at | 43 | 0.00815 |
| 1558216_at | 43 | 0.008172 |
| 228555_at | 43 | 0.008178 |
| 228396_at | 43 | 0.008188 |
| 241897_at | 43 | 0.008196 |
| 226488_at | 43 | 0.008201 |
| 215418_at | 43 | 0.008202 |
| 210910_s_at | 43 | 0.008216 |
| 210136_at | 43 | 0.008226 |
| 201791_s_at | 43 | 0.008236 |
| 219879_s_at | 43 | 0.00825 |
| 210553_x_at | 43 | 0.008271 |
| 218916_at | 43 | 0.008275 |
| 226132_s_at | 43 | 0.008278 |
| 210058_at | 43 | 0.008279 |
| 233599_at | 43 | 0.008282 |
| 1558275_at | 43 | 0.00829 |
| 212541_at | 43 | 0.008297 |
| 237563_s_at | 43 | 0.0083 |
| 218797_s_at | 43 | 0.008304 |
| 210707_x_at | 43 | 0.008311 |
| 201744_s_at | 43 | 0.00833 |
| 203420_at | 43 | 0.008348 |
| 212944_at | 43 | 0.008355 |
| 236122_at | 43 | 0.008419 |
| 226727_at | 43 | 0.008422 |
| 1569150_x_at | 43 | 0.008423 |
| 204952_at | 43 | 0.008431 |
| 227597_at | 43 | 0.008436 |
| 201936_s_at | 43 | 0.008445 |
| 209494_s_at | 43 | 0.008481 |
| 242593_at | 43 | 0.008496 |
| 236924_at | 43 | 0.008496 |
| 223811_s_at | 43 | 0.008516 |
| 231057_at | 43 | 0.008531 |
| 221919_at | 43 | 0.008532 |
| 218300_at | 43 | 0.008545 |
| 202275_at | 43 | 0.00855 |
| 223506_at | 43 | 0.008567 |
| 224952_at | 43 | 0.008572 |
| 221664_s_at | 43 | 0.008584 |
| 213322_at | 43 | 0.008588 |
| 1568617_a_at | 43 | 0.008589 |
| 216347_s_at | 43 | 0.008595 |
| 219185_at | 43 | 0.008603 |
| 226314_at | 43 | 0.008609 |
| 238448_at | 43 | 0.008613 |
| 219400_at | 43 | 0.008616 |
| 203489_at | 43 | 0.008623 |
| 229156_s_at | 43 | 0.008642 |
| 206746_at | 43 | 0.00865 |
| 203513_at | 43 | 0.008681 |
| 214152_at | 43 | 0.008683 |
| 225070_at | 43 | 0.008695 |
| 1569149_at | 43 | 0.008698 |
| 223222_at | 43 | 0.00871 |
| 216883_x_at | 43 | 0.008712 |
| 222096_x_at | 43 | 0.008723 |
| 222436_s_at | 43 | 0.008729 |
| 201600_at | 43 | 0.008741 |
| 206687_s_at | 43 | 0.00875 |
| 223383_at | 43 | 0.008762 |
| 1555788_a_at | 43 | 0.008764 |
| 48117_at | 43 | 0.008773 |
| 219027_s_at | 43 | 0.008775 |
| 208696_at | 43 | 0.008778 |
| 200757_s_at | 43 | 0.008781 |
| 218792_s_at | 43 | 0.008784 |
| 1552740_at | 43 | 0.00879 |
| 1553015_a_at | 43 | 0.008792 |
| 242576_x_at | 43 | 0.008796 |
| 235775_at | 43 | 0.008812 |
| 224935_at | 43 | 0.008816 |
| 227348_at | 43 | 0.008819 |
| 235535_x_at | 43 | 0.008821 |
| 229563_s_at | 43 | 0.008826 |
| 239642_at | 43 | 0.008845 |
| 1556338_at | 43 | 0.008849 |
| 230843_at | 43 | 0.008864 |
| 218137_s_at | 43 | 0.008866 |
| 225241_at | 43 | 0.008871 |
| 238871_at | 43 | 0.008885 |
| 206561_s_at | 43 | 0.008901 |
| 239067_s_at | 43 | 0.008911 |
| 56829_at | 43 | 0.008933 |
| 225427_s_at | 43 | 0.008943 |
| 221689_s_at | 43 | 0.008947 |
| 40465_at | 43 | 0.008954 |
| 214526_x_at | 43 | 0.008959 |
| 232478_at | 43 | 0.008965 |
| 229933_at | 43 | 0.008968 |
| 201210_at | 43 | 0.008972 |
| 213946_s_at | 43 | 0.008981 |
| 222640_at | 43 | 0.008986 |
| 232646_at | 43 | 0.008995 |
| 206767_at | 43 | 0.008997 |
| 214988_s_at | 43 | 0.009 |
| 225997_at | 43 | 0.009032 |
| 209067_s_at | 43 | 0.009037 |
| 202161_at | 43 | 0.009041 |
| 202765_s_at | 43 | 0.009046 |
| 216568_x_at | 43 | 0.009055 |
| 227754_at | 43 | 0.009072 |
| 242549_at | 43 | 0.009075 |
| 218793_s_at | 43 | 0.009088 |
| 1555269_a_at | 43 | 0.009102 |
| 201581_at | 43 | 0.009105 |
| 205146_x_at | 43 | 0.009106 |
| 241938_at | 43 | 0.009137 |
| 204545_at | 43 | 0.009144 |
| 226484_at | 43 | 0.009149 |
| 211113_s_at | 43 | 0.009154 |
| 226479_at | 43 | 0.009162 |
| 236704_at | 43 | 0.009169 |
| 242559_at | 43 | 0.009172 |
| 228170_at | 43 | 0.009183 |
| 203482_at | 43 | 0.00919 |
| 208986_at | 43 | 0.009199 |
| 204198_s_at | 43 | 0.009199 |
| 200851_s_at | 43 | 0.009202 |
| 208929_x_at | 43 | 0.009207 |
| 239283_at | 43 | 0.009212 |
| 219801_at | 43 | 0.009215 |
| 222094_at | 43 | 0.00922 |
| 236487_at | 43 | 0.009233 |
| 234788_x_at | 43 | 0.009261 |
| 225642_at | 43 | 0.009261 |
| 242297_at | 43 | 0.009265 |
| 203102_s_at | 43 | 0.009311 |
| 1564653_s_at | 43 | 0.009321 |
| 221616_s_at | 43 | 0.009322 |
| 219475_at | 43 | 0.009328 |
| 1569322_at | 43 | 0.00933 |
| 201394_s_at | 43 | 0.009338 |
| 216028_at | 43 | 0.009363 |
| 235000_at | 43 | 0.009377 |
| 221814_at | 43 | 0.009389 |
| 222460_s_at | 43 | 0.009398 |
| 241713_s_at | 43 | 0.0094 |
| 236974_at | 43 | 0.009404 |
| 233321_x_at | 43 | 0.009463 |
| 221858_at | 43 | 0.009472 |
| 203777_s_at | 43 | 0.009499 |
| 214473_x_at | 43 | 0.00952 |
| 1554677_s_at | 43 | 0.009522 |
| 216765_at | 43 | 0.009534 |
| 204344_s_at | 43 | 0.009536 |
| 235275_at | 43 | 0.009549 |
| 216038_x_at | 43 | 0.009555 |
| 212946_at | 43 | 0.009565 |
| 1559067_a_at | 43 | 0.009577 |
| 240326_at | 43 | 0.00958 |
| 227448_at | 43 | 0.00958 |
| 201559_s_at | 43 | 0.009594 |
| 218445_at | 43 | 0.009621 |
| 210320_s_at | 43 | 0.009643 |
| 221629_x_at | 43 | 0.009649 |
| 204732_s_at | 43 | 0.009664 |
| 212618_at | 43 | 0.00967 |
| 229733_s_at | 43 | 0.009674 |
| 229104_s_at | 43 | 0.009674 |
| 218001_at | 43 | 0.009682 |
| 235131_at | 43 | 0.009692 |
| 204461_x_at | 43 | 0.009693 |
| 224605_at | 43 | 0.009701 |
| 46323_at | 43 | 0.009706 |
| 206416_at | 43 | 0.009721 |
| 204675_at | 43 | 0.009724 |
| 226616_s_at | 43 | 0.009731 |
| 226203_at | 43 | 0.009732 |
| 218780_at | 43 | 0.009749 |
| 224643_at | 43 | 0.009768 |
| 217904_s_at | 43 | 0.009792 |
| 209316_s_at | 43 | 0.009816 |
| 220093_at | 43 | 0.00982 |
| 222905_s_at | 43 | 0.009824 |
| 213300_at | 43 | 0.009835 |
| 218734_at | 43 | 0.009844 |
| 222630_at | 43 | 0.009848 |
| 205239_at | 43 | 0.009864 |
| 212995_x_at | 43 | 0.009871 |
| 213213_at | 43 | 0.009892 |
| 234949_at | 43 | 0.009898 |
| 230353_at | 43 | 0.009915 |
| 1557675_at | 43 | 0.009917 |
| 1557948_at | 43 | 0.009927 |
| 229946_at | 43 | 0.009932 |
| 214429_at | 43 | 0.009943 |
| 227375_at | 43 | 0.009944 |
| 233490_at | 43 | 0.009964 |
| 203533_s_at | 43 | 0.009972 |
| 201486_at | 43 | 0.009973 |
| 226330_s_at | 43 | 0.009979 |
| 212191_x_at | 43 | 0.009984 |

### Method of Generating Predictive Model

Some embodiments relate to a method of generating a predictive model for assessing a subject's response to a chemotherapy drug, comprising obtaining expression levels of a plurality of biomarkers in at least one cancer cell line; determining an inhibition activity of the chemotherapy drug on the plurality of cancer cell lines; determining a relationship between the expression levels of the plurality of biomarkers and the inhibition activity of the chemotherapy drug; generating the predictive model based on the relationship between the expression levels of the plurality of biomarkers and the inhibition concentration of the chemotherapy drug.

In some embodiments, determining the relationship between the expression levels of the plurality of biomarkers and the inhibition activity of the chemotherapy drug comprises selecting a first set of biomarkers using one or more mathematical techniques. In some embodiments, the mathematical techniques can be an ensemble learning technique, a predictor screening technique, linear regression analysis, and/or higher order regression analysis. In some embodiments, the mathematical techniques can be bootstrap Forest Partitioning technique, a predictor screening technique, linear regression analysis, and/or higher order regression analysis. In some embodiments, the ensemble learning technique can be a random forest method. In some embodiments, the ensemble learning technique can be a bootstrap forest model. In some embodiments, the ensemble learning technique can be a bootstrap forest partitioning technique.

In some embodiments, determining the relationship between the expression levels of the plurality of biomarkers and the inhibition activity of the chemotherapy drug comprises ranking the plurality of biomarkers based on a predictive score generated using a bootstrap Forest Partitioning technique, a predictor screening technique; or utilizing linear regression analysis or higher order regression analysis.

In some embodiments, the method described herein includes selecting a second set of biomarkers from the first set of biomarkers using one or more ensemble learning methods for classification and regression. In some embodiments, the method described herein includes selecting a second set of biomarkers from the first set of biomarkers using one or more mathematical techniques.

In some embodiments, the method described herein includes selecting a second set of biomarkers from the first set of biomarkers using a bootstrap Forest Partitioning technique. In some embodiments, the method described herein includes selecting a second set of biomarkers from the first set of biomarkers using a mathematical technique. In some embodiments, the method described herein includes selecting a second set of biomarkers from the first set of biomarkers using an ensemble learning technique, a predictor screening technique, linear regression analysis, and/or higher order regression analysis.

In some embodiments, the biomarker is an mRNA associated with one or more probesets; and the method further comprises ranking the probesets based on the correlation of the associated biomarker with the inhibition activity of the chemotherapy drug and keeping only the probesets with the highest rank for each associated biomarker for the selecting process.

In some embodiments, the method described herein includes using the second set of biomarkers to generate a predictive model for classifying the subject's response as active or inactive to the chemotherapy drug.

In some embodiments, the method described herein includes selecting one or more biomarkers based on the rank of the predictive score and generating the predictive model using the selected one or more biomarkers.

In some embodiments, the predictive model is selected from a neural network, a non-neural network model, or a combination thereof. In some embodiments, the method described herein includes the predictive model is selected from one or more one-layer Tan H multimode fit neural network model, one or more non-neural binomial logistic model, or a combination thereof. In some embodiments, the method described herein includes the predictive model is generated using an artificial intelligence software, a program or a technology for deriving predictive functions.

In some embodiments, the method described herein includes validating the predictive model using a set of validation data.

In some embodiments, the biomarker is an mRNA associated with one or more probesets listed in Table 1, Table 2, or Table 4.

In some embodiments, determining the inhibition activity of the chemotherapy drug comprises measuring the inhibition activity after treating the cancer cell lines with a media containing the chemotherapy drug.

In some embodiments, the method described herein includes treating the cancer cell lines with the media containing the chemotherapy drug for about 12 hours to 36 hours followed by treating the cancer cell lines with a media without the chemotherapy drug prior to measuring the inhibition activity. In some embodiments, the method described herein includes treating the cancer cell lines with the media containing the chemotherapy drug for about 12 hours to 36 hours followed by treating the cancer cell lines with a media without the chemotherapy drug for about 48 hours to about 96 hours prior to measuring the inhibition activity. In some embodiments, the method described herein includes treating the cancer cell lines with the media containing the chemotherapy drug for about 24 hours followed by treating the cancer cell lines with a media without the chemotherapy drug for about 72 hours prior to measuring the inhibition activity.

In some embodiments, the method described herein includes setting a threshold inhibition activity and assigning the inhibition activity of the chemotherapy drug on the plurality of cancer cell lines as active or inactive based on the threshold inhibition activity. In some embodiments, the inhibition activity is measured based on an inhibition concentration of the chemotherapy drug producing 50%, 60%, 70%, 80%, 80%, or 90% of the maximum inhibition effect (IC50, IC60, IC70, IC80, or IC90 value). In some embodiments, the inhibition activity is measured based on an IC50 value. In some embodiments, the inhibition activity is measured based on an IC60 value. In some embodiments, the inhibition activity is measured based on an IC70 value. In some embodiments, the inhibition activity is measured based on an IC80 value. In some embodiments, the inhibition activity is measured based on an IC90 value.

In some embodiments, the chemotherapy drug is classified as responsive when the measured IC is lower than or equal to about 50, 40, 30, 20, 15, 10, 9, 8, 7, 6, 5, 4, 3, 2, 1, 0.5, or 0.1 μM, and the IC can be IC50, IC60, IC70, IC80, or IC90. In some embodiments, the chemotherapy drug is classified as responsive when the IC₇₀ or IC₅₀ is lower than or equal to about 50, 40, 30, 20, 15, 10, 9, 8, 7, 6, 5, 4, 3, 2, 1 μM. In some embodiments, the chemotherapy drug is classified as nonresponsive when the measured IC is higher than 0.5, 1, 2, 3, 4, 5, 6, 7, 8, 9, 10, 20, 30, 40, 50, 60, 80, or 100 μM and the IC can be IC50, IC60, IC70, IC80, or IC90. In some embodiments, the chemotherapy drug is classified as responsive when the IC₇₀ or IC₅₀ is greater than about 0.5, 1, 2, 3, 4, 5, 6, 7, 8, 9, 10, 20, 30, 40, 50, 60, 80, or 100 μM.

In some embodiments, the method described herein includes classifying the inhibition activity and the ordinal activity status of the model or cell line as active or inactive based on the measured IC₅₀, IC₆₀, IC₇₀, IC₈₀, or IC₉₀ value after comparing with a threshold value.

### EXAMPLES

### Example 1

Working stock solution of the test compounds (Plinabulin, Docetaxel and Paclitaxel) were prepared in DMSO at a concentration of 3.14 (Plinabulin) or 3.3 mM (Docetaxel and Paclitaxel), and small aliquots were stored at −20° C. On each day of an experiment, a frozen aliquot of the working stock solutions was thawed and stored at room temperature prior to and during treatment.

All liquid handling steps were done by the Tecan Freedom EVO 200 platform. First, serial half-log dilutions of the DMSO working stock solution were made in DMSO. The DMSO dilutions were then diluted 1:22 into cell culture medium in an intermediate dilution plate. Finally, 10 μl taken from the intermediate dilution plate was transferred to 140 μl/well of the final assay plate. Thus, the DMSO serial dilutions were diluted 1:330 with cell culture medium, and the DMSO concentration in the assay was 0.3% v/v in all wells, including untreated control wells.

Tumor Cell Lines: The human tumor cell lines used in this study were derived from lung cancer, breast cancer, prostate cancer, ovarian cancer, and central nervous system cancer/glioblastoma (Table 3).

TABLE 3
Human Tumor Cell Lines Utilized for Potency Screening

| Cancer | Plinabulin | | |
|---|---|---|---|
| Type | Activity | Cell Lines | Agents Tested |
| CNS | Active | A-172, SF-539, SNB-78, | Plinabulin and |
| | | U-251, U-87MG | Colchcine |
| Lung | Active | Calu-, LXFL1121, NCI- | Plinabulin and |
| | | H460 | Docetaxel |
| Lung | Inactive | LXFA289, LXFA526, | Plinabulin and |
| | | LXFA629, LXFA983, | Docetaxel |
| | | A549, HOP-62, NCI- | |
| | | H322M, NCI-H226, SK- | |
| | | MES-1, A427, NCI-H1299, | |
| | | H2171, SCLC-21H | |
| Breast | Active | CAL-51, HS578T, JIMT-1, | Plinabulin and |
| | | MCF10A, MX1 | Docetaxel |
| Breast | Inactive | MAXFTN401, BT-474, | Plinabulin and |
| | | HCC-1937, MCF7, MDA- | Docetaxel |
| | | MB-231 | |
| Ovarian | Active | A2780, EFO-21, EFO-27, | Plinabulin and |
| | | OVCAR3 | Paclitaxel |
| Ovarian | Inactive | OVXF899, OVXF1023 | Plinabulin and |
| | | | Paclitaxel |
| Prostate | Active | 22Rv1, DU-145, LNCaP, | Plinabulin and |
| | | PC-3M | Docetaxel |
| Prostate | Inactive | PC-3 | Plinabulin and |
| | | | Docetaxel |

Cell lines were either provided by the NCI (Bethesda, Md.), or were purchased from ATCC (Rockville, Md.), DSMZ (Braunschweig, Germany), CLS (Cell Line Service, Heidelberg, Germany), or ECACC (European collection of authenticated cell cultures). Authenticity of cell lines was proven at the DSMZ by STR (short tandem repeat) analysis, a PCR based DNA-fingerprinting methodology.

Cell lines were routinely passaged once or twice weekly and maintained in culture for up to 20 passages. They were grown at 37° C. in a humidified atmosphere with 5% CO₂ in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.05 mg/mL gentamicin (Life Technologies, Karlsruhe, Germany).

CellTiter-Blue® Assay: The CellTiter-Blue® Cell Viability Assay (#G8081, Promega) was performed according to manufacturer's instructions. Briefly, cells were harvested from exponential phase cultures, counted and plated in 96-well flat-bottom microtiter plates at a cell density of 4,000 to 60,000 cells/well dependent on the cell line's growth rate. The individual seeding density for each cell line ensures exponential growth conditions over the whole or at least the bigger part of the treatment period. After a 24 h recovery period to allow the cells to resume exponential growth, 10 μl of culture medium (six control wells/plate) or of culture medium with test compounds were added. Compounds were applied at 10 concentrations in duplicate in half-log increments up to 10 μM (plinabulin, docetaxel, colchicine and paclitaxel) or 9.5 μM (plinabulin) and cells were treated for a period of 24 hours. After the initial application of the compounds for 24 hours, the compound-containing media was exchanged to media without compounds and incubation was continued for a further 48 hours until read-out. After treatment of cells, 20 μl/well CellTiter-Blue® reagent was added. Following incubation with CellTiter-Blue® reagent for up to four hours, fluorescence (FU) was measured by using the Enspire Multimode Plate Reader (excitation λ=570 nm, emission λ=600 nm). For calculations of cytotoxicity, the mean values of duplicate/sextuplicate (untreated control) data were used.

Cytotoxicity Data Evaluation: An assay was considered fully evaluable if the following quality control criteria were fulfilled:

Z′-factor calculated within the assay plate

control/background ratio >3.0

coefficient of variation in the growth control wells ≤30%

Drug effects were expressed in terms of the percentage of the fluorescence signal, obtained by comparison of the mean signal in the treated wells with the mean signal of the untreated controls (expressed by the test-versus-control value, T/C-value [%]):

$$\frac{T}{C} [ \% ] = \frac{mean fluorescence {signal}_{treated group}}{mean fluorescence {signal}_{control group}} \cdot 100$$

The absolute IC₇₀ value gives the concentration of the test compound that achieves T/C=30% at the end of the 72 hour culture period. Calculation was performed by 4 parameter non-linear curve fit (Oncotest Data Warehouse Software). If an IC₇₀ value could not be determined within the examined dose range (because a compound lacked activity), the highest concentration studied was indicated: 10 μM (plinabulin, docetaxel, colchicine and paclitaxel) or 9.5 μM (plinabulin).

Array mRNA Expression: Gene expression (mRNA) was evaluated utilizing an Affymetrix HGU133 Plus 2.0 array according to Oncotest standard practices. This array uses sequence-specific hybridization between a fixed set of DNA Probes (probeset) and a labeled RNA target. Log 2 transformed Affymetrix gene probeset signal values were preprocessed with the GeneChip robust multi-array average analysis algorithm and then utilized for statistical analyses below.

Identification of Response Biomarkers and Predictive Algorithms: Predictor-TTest Method: Utilizing JMP 14.1 Statistical software (from SAS), all probeset expression values were ranked together as predictors of ordinal response using a Bootstrap Forest Partitioning technique utilizing 100 trees. From the top 200 predictor probesets, 40 “HIT” probesets were identified (one per gene) that also exhibited differential expression in Active versus Inactive cell lines (p<0.01, T-test). For probesets with gene annotation, only the probeset for each gene with the highest Jetset score was utilized for model development (Li et al., 2011).

Correlation-Predictor Method: Utilizing JMP 14.1 Statistical software, all probeset expression values were tested with the Response Screening function by calculating the correlation coefficient and p-value for each probeset versus the plinabulin IC₇₀ and then sorting based on the p-values. Probesets with p<0.01 in this analysis were selected and run through the Predictor Screening function 3 times (1000 trees). 91 probesets ranked in the top 100 for 2-3 runs were then selected and for those with an average rank <50 (low score=high rank) and not already picked up with the Predictor-TTest method above, the gene annotation was evaluated. 16 “HIT” probesets that were non-annotated or had the highest Jetset score for each identified gene, and had differential expression between plinabulin active versus inactive cell lines (p<0.01; ANOVA), were selected.

The 56 HIT probesets from above were then ranked 4 times as predictors in JMP, utilizing two different orders of probeset input into the Predictor Screening method (1000 trees). Finally, from a selection of the HIT probesets, multiple one layer Tan H multimode fit neural network models were constructed to identify plinabulin responding cell lines with confidence, in both a training and validation set. Binomial logistic regression models were also developed to predict plinabulin response as a function of select HIT probeset values.

### Results

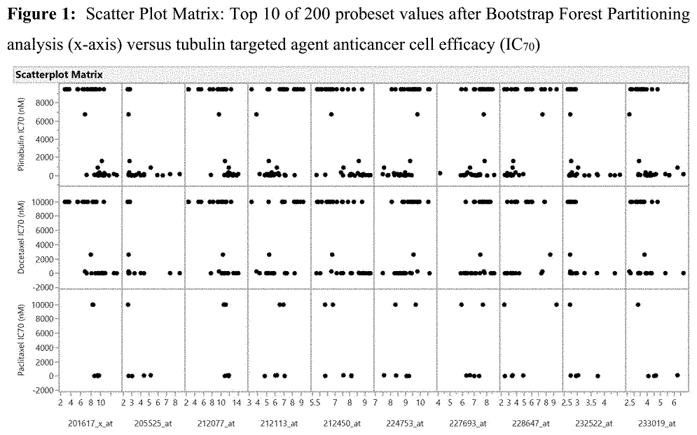

Active Versus Inactive Classification: Utilizing JMP software, final values from 54,675 probesets in the Affymetrix HGU133 Plus 2.0 array were evaluated as predictors for plinabulin IC₇₀. It is seen in FIG. 1 that the IC₇₀ values for plinabulin, as well as those for paclitaxel and docetaxel, plotted versus the expression value for the top 10 ranked predictor probesets, were essentially grouped into those that are active (IC₇₀<1 □M) and those that were inactive (IC₇₀>1 □M, and usually >10 □M). For this reason, cell lines were assigned an ordinal variable value of Active or Inactive, as shown in Table 3, rather than focusing on the IC50 as is commonly done.

Selection of HIT Predictor Genes/Probesets With Predictor-TTest Method: The probesets ranked among the top 200 predictors were compared by t-test in Active versus Inactive tumor cell lines. For those reaching p<0.05 (probeset value differed in plinabulin Active and Inactive cell lines at the 5% level, unadjusted for multiple comparisons), the annotated genes for these probesets, if available, were noted. Next, all of the probesets in the array that are mapped to the same noted genes were identified. Jetset scoring methods to assess each probeset for specificity, splice isoform coverage, and robustness against transcript degradation have been shown to be valuable tools in assessing the value of each probeset, in particular correlating with protein expression (Li et al 2011). At this point therefore, the probeset with the highest Jetset score that mapped to each noted gene, with a p value <0.01 for Active versus Inactive values, was selected for final ranking of its predictive ability. In addition, probesets without a mapped gene, with a p value <0.01 for plinabulin Active versus Inactive values, were also selected. These 40 total Predictor TTest method selected probesets (HITs), and mapped genes if available, are listed in Table 4.

Selection of HIT Predictor Genes/Probesets With Correlation-Predictor Method: Probesets with correlation p-values <0.01 versus plinabulin IC₇₀ were run 3 times through the Predictor Screening process in JMP for their ability to predict plinabulin Active versus Inactive. 91 probesets ranked in the top 100 for 2-3 runs were then selected and for those with an average rank <50 (low score=high rank) and not already picked up with the Predictor-TTest method above, the gene annotation was evaluated. 16 “HIT” probesets that were non-annotated or had the highest Jetset score for each identified gene, and had differential expression between plinabulin active versus inactive cell lines (p<0.01; ANOVA), were selected.

The 56 HITS from above provide evidence that the expression of each of the noted genes (mRNA or protein), or the calculated array value for the indicated probesets, on samples from patients containing tumor cells, has the potential to predict benefit from plinabulin. Moreover since certain probesets marked with an asterisk in Table 4, had differential expression (p<0.05, even with the reduced number of cell lines tested with docetaxel) in tumor cell lines that were Active versus Inactive for docetaxel, when the ordinal value of docetaxel activity was assigned in the same way as done for plinabulin, the generated data indicates the expression of these marked genes and probeset signals may be used to predict tubulin targeted drug activity in general. The accuracy of using any one gene will be limited by the overlap in the probeset signals in the Active and Inactive groups (e.g. see FIG. 1), and by the variability inherent in the measurement of only a single gene in each sample. Thus the use of data from multiple probesets/genes may be necessary to reach a confidence in activity assignment that has utility for making treatment decisions in the clinic.

TABLE 4
Human Tumor Cell Lines Utilized for Potency Screening

| | | | p value: | | p value: |
|---|---|---|---|---|---|
| | | | Plinabulin | Avg | Docetaxed |
| | | Mapped Gene | Active vs | Predictor | Active vs |
| Probeset | Method | Symbol | Inactive | Rank | Inactive |
| 212077_at | Both | CALD1 | 0.00010 | 1.00 | 0.00130* |
| 215983_s_at | Both | UBXN8 | 0.00010 | 2.50 | 0.00680* |
| 224753_at | Both | CDCA5 | 0.00010 | 2.50 | 0.14533 |
| 223641_at | Correl-Pred | Unknown | 0.0001 | 5.75 | 0.0821 |
| 226416_at | Correl-Pred | ERI1 | 0.0013 | 6.50 | 0.0633 |
| 217667_at | Correl-Pred | SEC14L1P1 | 0.0001 | 7.25 | 0.054 |
| 21245O_at | Both | SECISBP2L/SLAN | 0.00010 | 7.25 | 0.01110* |
| 227693_at | Correl-Pred | WDR20 | 0.0012 | 7.50 | 0.0668 |
| 213880_at | Both | LGR5 | 0.00550 | 8.50 | 0.10080 |
| 201346_at | Correl-Pred | ADIPOR2 | 0.0022 | 10.75 | 0.2266 |
| 238550_at | Correl-Pred | RUFY2 | 0.0038 | 11.00 | 0.32 |
| 221729_at | Correl-Pred | COL5A2 | 0.0001 | 11.25 | 0.14 |
| 213077_at | Correl-Pred | YTHDC2 | 0.0005 | 12.25 | 0.0202* |
| 200809_x_at | Both | RPL12 | 0.00040 | 14.75 | 0.00910* |
| 213278_at | Correl-Pred | MTMR9 | 0.0003 | 18.50 | 0.1228 |
| 238342_at | Correl-Pred | Unknown | 0.0066 | 18.50 | 0.29 |
| 232522_at | Both | Unknown | 0.00350 | 18.50 | 0.07880 |
| 224755_at | Both | TM9SF3 | 0.00070 | 18.75 | 0.01530* |
| 205428_s_at | Both | CALB2 | 0.00040 | 21.00 | 0.1930 |
| 1559332_at | Predictor- | Unknown | 0.00260 | 21.25 | 0.15180 |
| | Ttest | | | | |
| 235071_at | Both | WDR92 | 0.00130 | 21.25 | 0.60460 |
| 209549_s_at | Correl-Pred | DGUOK | 0.0002 | 22.75 | 0.0115* |
| 201533_at | Predictor- | CTNNB1 | 0.00100 | 23.00 | 0.08680 |
| | Ttest | | | | |
| 200895_s_at | Correl-Pred | FKBP4 | 0.0001 | 23.25 | 0.0007* |
| 225217_s_at | Both | BRPF3 | 0.00060 | 23.25 | 0.01660* |
| 221081_s_at | Correl-Pred | DENND2D | 0.0015 | 23.50 | 0.0147* |
| 209656_s_at | Both | TMEM47 | 0.00100 | 26.00 | 0.09950 |
| 202649_x_at | Both | RPS19 | 0.00290 | 27.25 | 0.10010 |
| 214862_x_at | Both | Unknown | 0.00090 | 28.00 | 0.11510 |
| 220525_s_at | Both | AUP1 | 0.00040 | 29.75 | 0.00230* |
| 229022_at | Both | ZFX | 0.00090 | 30.50 | 0.28180 |
| 243801_x_at | Predictor- | MRPL30 | 0.00090 | 34.75 | 0.66250 |
| | Ttest | | | | |
| 202080_s_at | Predictor- | TRAK1 | 0.00160 | 35.25 | 0.11180 |
| | Ttest | | | | |
| 226488_at | Predictor- | RCCD1 | 0.00170 | 35.50 | 0.34910 |
| | Ttest | | | | |
| 235796_at | Correl-Pred | Unknown | 0.0013 | 36.50 | 0.0844 |
| 225725_at | Predictor- | ZMAT3 | 0.00090 | 37.25 | 0.04230* |
| | Ttest | | | | |
| 222821_s_at | Both | GEMIN7 | 0.00170 | 37.75 | 0.01230* |
| 217781_s_at | Predictor- | ZNF106 | 0.00120 | 38.00 | 0.66240 |
| | Tlest | | | | |
| 226848_at | Both | Unknown | 0.00010 | 39.00 | 0.00600* |
| 218146_at | Correl-Pred | GLT8D1 | 0.0046 | 39.25 | 0.0032* |
| 224619_at | Predictor- | CASC4 | 0.00100 | 42.25 | 0.03490* |
| | Ttest | | | | |
| 225086_at | Predictor- | FAM98B | 0.00270 | 42.50 | 0.18470 |
| | Ttest | | | | |
| 201268_at | Predictor- | NME1-NME2 | 0.00170 | 42.75 | 0.01130* |
| | Ttest | | | | |
| 226395_at | Both | HOOK3 | 0.00170 | 44.00 | 0.06070 |
| 229666_s_at | Correl-Pred | CSTF3 | 0.0056 | 44.50 | 0.0094* |
| 228603_at | Predictor- | ACTR3 | 0.00120 | 44.50 | 0.01230* |
| | Ttest | | | | |
| 233678_at | Predictor- | Unknown | 0.00660 | 45.50 | 0.03390* |
| | Ttest | | | | |
| 202029_x_at | Predictor- | RPL38 | 0.00160 | 46.00 | 0.27390 |
| | Ttest | | | | |
| 235031_at | Predictor- | Unknown | 0.00220 | 46.75 | 0.02650 |
| | Ttest | | | | |
| 200827_at | Predictor- | PLOD1 | 0.00840 | 46.75 | 0.08340 |
| | Ttest | | | | |
| 225185_at | Predictor- | MRAS | 0.00340 | 48.25 | 0.05500 |
| | Ttest | | | | |
| 1553193_at | Predictor- | ZNF441 | 0.00690 | 49.25 | 0.03630* |
| | Ttest | | | | |
| 205205_at | Predictor- | RELB | 0.00940 | 51.00 | 0.28240 |
| | Ttest | | | | |
| 203866_at | Both | NLE1 | 0.00800 | 54.75 | 0.07400 |
| 222096_x_at | Predictor- | Unknown | 0.00800 | 54.75 | 0.72760 |
| | Ttest | | | | |
| 223156_at | Predictor- | MRPS23 | 0.00990 | 55.50 | 0.06290 |
| | Ttest | | | | |

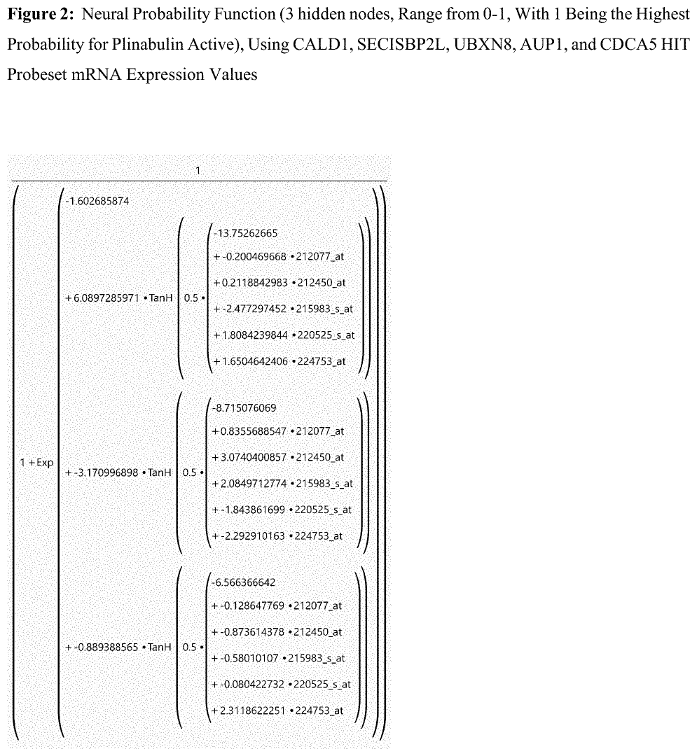

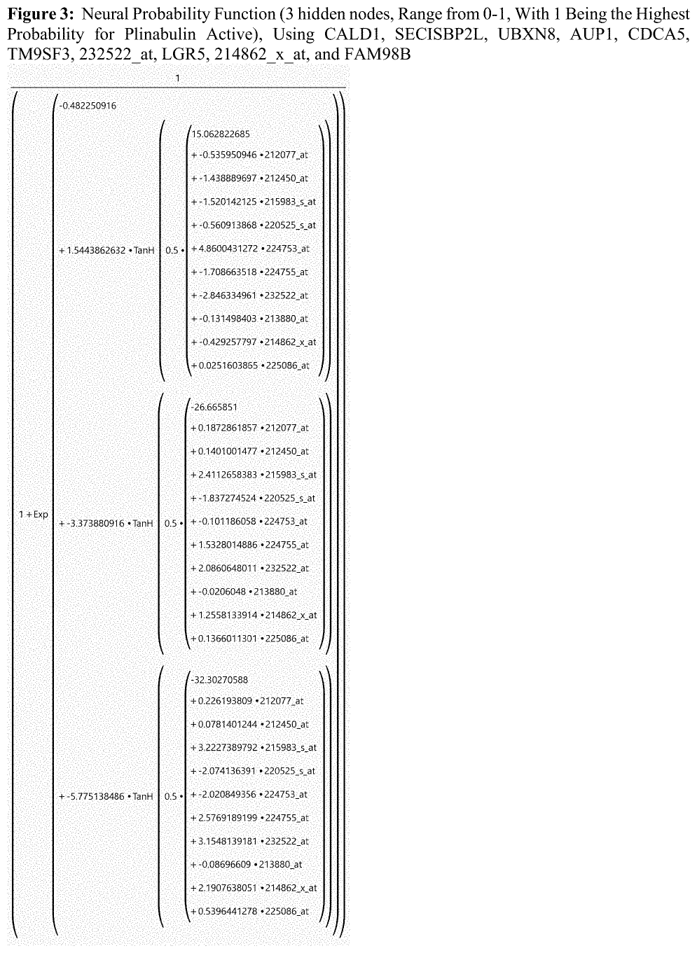

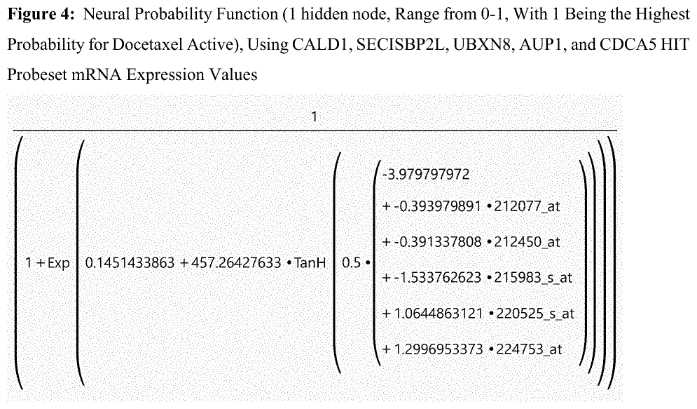

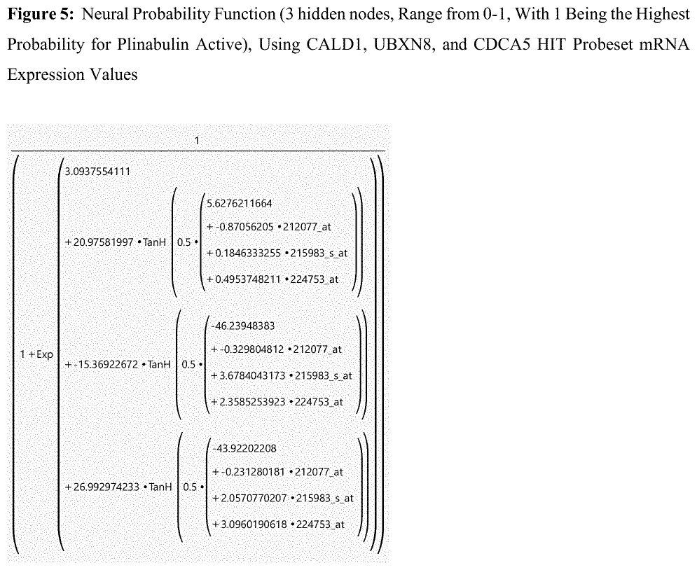

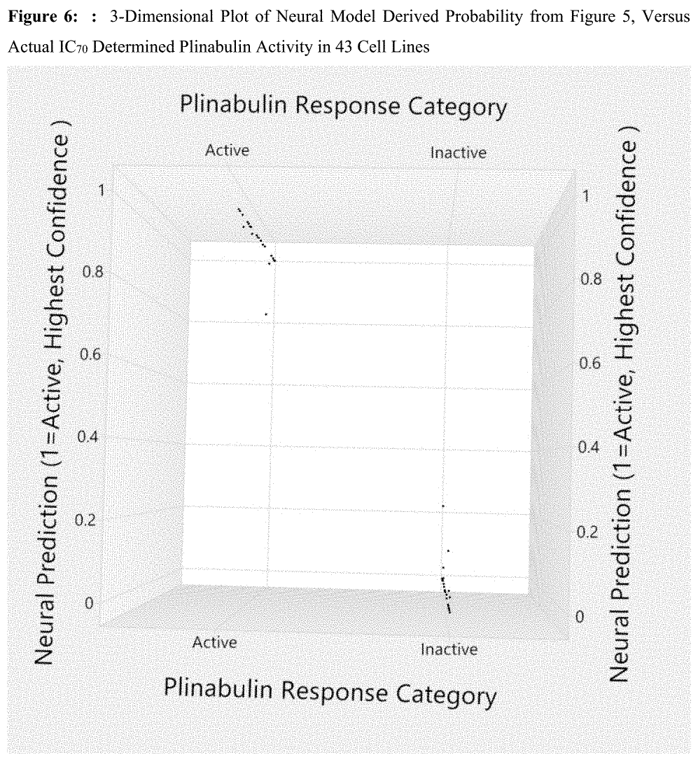

Predictive Algorithms Utilizing Data From Multiple Probesets: The 56 HIT probesets were ranked as predictors utilizing Bootstrap Forest Partitioning in JMP four times. The average ranking for each probeset is shown in Table 4. The method(s) used to discover the HIT probesets/genes are also listed. Selections of probesets were taken and used to construct multiple one layer Tan H multimode fit neural network models that identify plinabulin responding cell lines with confidence. Utilizing 5 top HIT predictor probesets, for example, and using ⅔ of the tumor cell lines as a training set (28 models) and the remaining 15 models as a validation set, with 3 hidden nodes, a model was developed (FIG. 2) that can predict the activity of plinabulin in the cell line models in the training set. In the validation set, plinabulin activity was predicted accurately for all models except for 1 model that was incorrectly classified as Active and 1 model that is incorrectly classified as Inactive. When 10 HITs were used instead, the developed model (FIG. 3) predicted plinabulin activity in the training set and validation set perfectly. Furthermore, when again the 5 top HIT predictor probesets were utilized in algorithm development, a simpler formula was generated and in this case the prediction of plinabulin response was perfect for both training and validation sets, when only 1 hidden node was utilized (FIG. 4). Finally, in some cases, even just 3 genes could be used to establish a neuronal probability model to predict response with high confidence (probabilities either close to 0 or close to 1) (FIGS. 5 and 6).

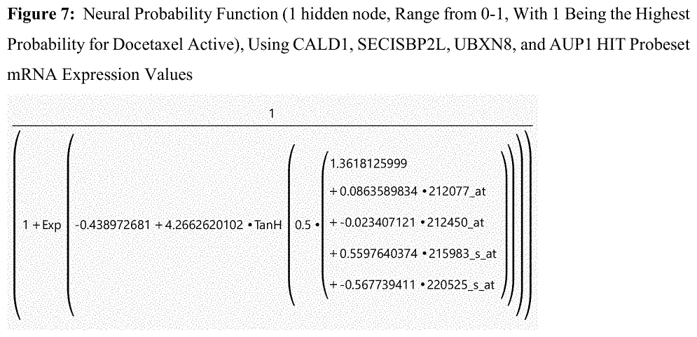

Importantly, even with the lower number of tumor cell lines tested for docetaxel activity, 4 HIT predictor probesets (CALD1, SECOISBP2L, UBXN8, and AUP1) could be used to develop a neural net algorithm in JMP with 1 hidden node, that predicted docetaxel activity accurately in 15 of 17 tumor cell lines in the training set and 9 of 10 tumor cell lines in the validation set (FIG. 7).

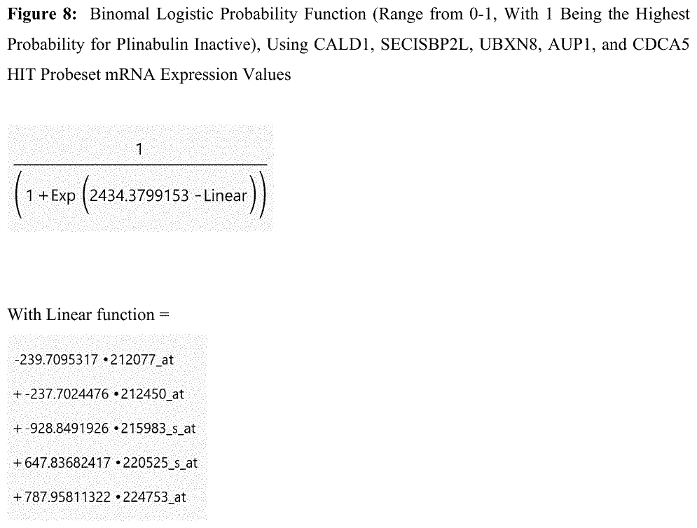

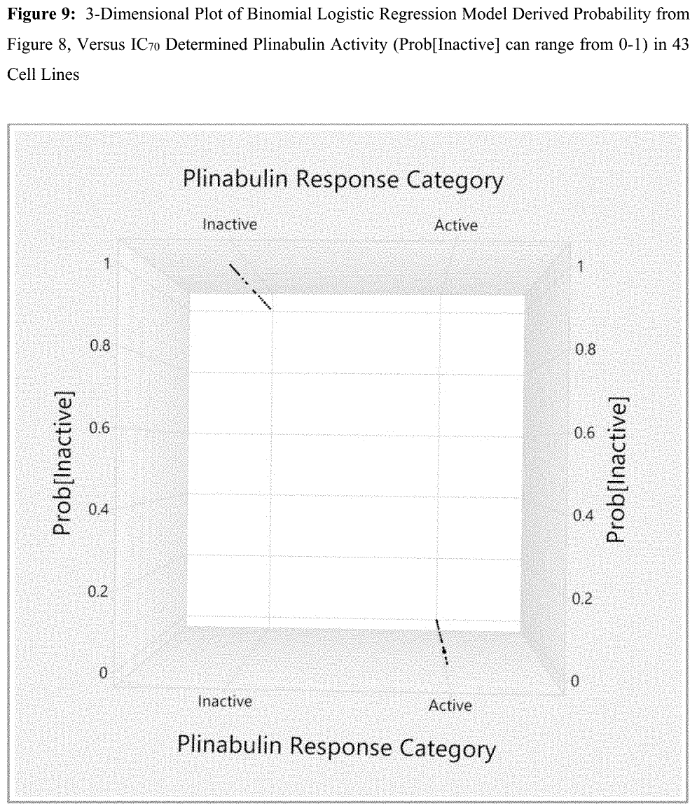

Tan H is the function utilized in the neural network model in JMP 14.1. Additional types of neural networks are in use and these too could be used to construct predictive algorithms utilizing the HIT probeset measurements. Non-neural binomial logistic regression modeling was also evaluated for predicting plinabulin activity utilizing all 43 models. The generated model reported in FIG. 8, perfectly predicts plinabulin activity for each of the tumor cell lines. Moreover, the probability scores for inactivity, which can range from 0 to 1, were essentially either 0 or 1 with nothing in between (FIG. 9).

The level of confidence in prediction for the above models and the models that can be similarly developed with the 56 HITs, utilizing only the expression measurements from less than 20, or less than 10, or less than 5 genes or probesets, is unexpected, novel, implementable, and potentially valuable to society.

The 56 HIT genes, or probesets without gene mapping, are novel biomarkers for predicting the ability of plinabulin, and tubulin targeted agents in general, to significantly reduce the number of cancer cells, or cancer burden. Beyond using single genes to predict response, our work establishes methods and algorithms for predicting potent anticancer effects for plinabulin and other tubulin targeted therapies with striking accuracy. These findings support the potential utility of these predictive biomarker strategies for selecting cancer patients most likely to derive significant benefit from plinabulin and other tubulin targeted agents, and also to enable those that are unlikely to respond to seek alternative therapies with potential benefit.

What is claimed is:

1. A method of treating a cancer, comprising:
selecting a subject responsive to treatment with a tubulin binding agent by determining an expression level of one or more biomarkers; and
administering an effective amount of the tubulin binding agent to the selected subject.

2. The method of claim 1, wherein the biomarker is an mRNA associated with one or more probesets.

3. The method of claim 1, wherein the biomarker is an mRNA associated with one or more probesets configured to identify an expression level in one or more cancer cell lines.

4. The method of claim 1, wherein the biomarker is an mRNA associated with one or more probesets listed in Table 1, Table 2, or Table 4.

5. The method of claim 1, wherein the biomarker is an mRNA.

6. The method of claim 1, wherein the biomarker is associated with an expression level of one or more genes selected from CALD1, UBXN8, CDCA5, ERI1, SEC14L1P1, SECISBP2L/SLAN, WDR20, LGR5, ADIPOR2, RUFY2, COL5A2, YTHDC2, RPL12, MTMR9, TM9SF3, CALB2, WDR92, DGUOK, CTNNB1, FKBP4, BRPF3, DENND2D, TMEM47, RPS19, AUP1, ZFX, MRPL30, TRAK1, RCCD1, ZMAT3, GEMIN7, ZNF106, GLT8D1, CASC4, FAM98B, NME1-NME2, HOOK3, CSTF3, ACTR3, RPL38, PLOD1, MARS, ZNF441, RELB, NLE1, MRPS23, and any combinations thereof.

7. The method of claim 1, wherein the biomarker is associated with an expression level of one or more genes selected from the group consisting of CALD1, SECISBP2L, UBXN8, AUP1, CDCA5, TM9SF3, LGR5, FAM98B, and combinations thereof.

8. The method of claim 1, wherein the biomarker is associated with an expression level of one or more genes selected from the group consisting of CALD1, SECISBP2L, UBXN8, AUP1, CDCA5, and any combinations thereof.

9. The method of claim 1, wherein the biomarker is associated with an expression level of one or more genes selected from the group consisting of CALD1, UBXN8, AUP1, CDCA5, and any combinations thereof.

10. The method of claim 1, wherein the biomarker is associated with an expression level of one or more genes selected from the group consisting of CALD1, SECISBP2L, UBXN8, AUP1, and any combinations thereof.

11. The method of any one of claims 1-10, comprising determining an expression score using the determined expression level of one or more biomarkers.

12. The method of any one of claims 1-10, comprising
obtaining a test sample derived from the subject;
determining an expression score using the determined expression level of the one or more biomarkers;
classifying the subject as responsive or non-responsive to the tubulin binding agent treatment based on the expression score.

13. The method of claim 12, wherein classifying the subject comprises classifying the subject as responsive or nonresponsive by comparing the expression score of a probeset or gene with a reference.

14. The method of any one of claims 1-13, wherein determining the expression score comprises using one or more predictive models.

15. The method of claim 14, wherein the predictive model is generated based on expression scores generated and/or threshold scores derived from one or more selected probesets or genes.

16. The method of claim 15, where the predictive model comprises one or more one-layer Tan H multimode fit neural network models, one or more non-neural binomial logistic model, or a combination thereof.

17. The method of any one of claims 1-16, wherein the expression level of the biomarker is measured using a probeset, microarray, quantitative PCR, or an immunoassay.

18. The method of any one of claims 1-17, wherein the tubulin binding agent is plinabulin.

19. The method of any one of claims 1-18, wherein the cancer is selected from central nervous system (CNS) lymphoma, lung cancer, breast cancer, ovarian cancer, and prostate cancer.

20. The method of claim 1, wherein the tubulin binding agent is co-administered with one or more chemotherapeutic agent.

21. The method of any one of claims 1-17, wherein the tubulin binding agent is a taxane.

22. The method of claim 21, wherein the taxane is docetaxel or paclitaxel.

23. The method of any one of claims 1-17, wherein the tubulin binding agent is a Vinca site binder.

24. The method of claim 23, wherein the tubulin binding agent is vinblastine or vincristine.

25. A method of generating a predictive model for assessing a subject's response to a chemotherapy drug, comprising:
obtaining expression levels of a plurality of biomarkers in at least one cancer cell line;
determining an inhibition activity of the chemotherapy drug on the plurality of cancer cell lines;
determining a relationship between the expression levels of the plurality of biomarkers and the inhibition activity of the chemotherapy drug;
generating the predictive model based on the relationship between the expression levels of the plurality of biomarkers and the inhibition concentration of the chemotherapy drug.

26. The method of claim 25, wherein determining the relationship between the expression levels of the plurality of biomarkers and the inhibition activity of the chemotherapy drug comprises selecting a first set of biomarkers using an ensemble learning method, a predictor screening technique, linear regression analysis, and/or higher order regression analysis.

27. The method of claim 25, wherein determining the relationship between the expression levels of the plurality of biomarkers and the inhibition activity of the chemotherapy drug comprises selecting a first set of biomarkers using a bootstrap Forest Partitioning technique, a predictor screening technique, linear regression analysis, and/or higher order regression analysis.

28. The method of claim 26, comprising selecting a second set of biomarkers from the first set of biomarkers using one or more ensemble learning methods for classification and regression.

29. The method of claim 28, wherein the ensemble learning method is a bootstrap Forest Partitioning technique.

30. The method of any one of claims 25 to 29, wherein the biomarker is an mRNA associated with one or more probesets; and the method further comprises ranking the probesets based on the correlation of the associated biomarker with the inhibition activity of the chemotherapy drug and keeping only the probesets with the highest rank for each associated biomarker for the selecting process.

31. The method of claim 28, comprising using the second set of biomarkers to generate a predictive model for classifying the subject's response as active or inactive to the chemotherapy drug.

32. The method of claim 25, wherein the predictive model is selected from a neural network, a non-neural network model, or a combination thereof.

33. The method of claim 25, wherein the predictive model is selected from one or more one-layer Tan H multimode fit neural network model, one or more non-neural binomial logistic model, or a combination thereof.

34. The method of claim 25, wherein the predictive model is generated using an artificial intelligence software, a program or a technology for deriving predictive functions.

35. The method of claim 25, comprising validating the predictive model using a set of validation data.

36. The method of claim 25, the biomarker is an mRNA associated with one or more probesets listed in Table 1, Table 2, or Table 4.

37. The method of claim 36, wherein the biomarker is an mRNA.

38. The method of claim 25, wherein is biomarker is associated with an expression level of one or more genes selected from CALD1, UBXN8, CDCA5, ERI1, SEC14L1P1, SECISBP2L/SLAN, WDR20, LGR5, ADIPOR2, RUFY2, COL5A2, YTHDC2, RPL12, MTMR9, TM9SF3, CALB2, WDR92, DGUOK, CTNNB1, FKBP4, BRPF3, DENND2D, TMEM47, RPS19, AUP1, ZFX, MRPL30, TRAK1, RCCD1, ZMAT3, GEMIN7, ZNF106, GLT8D1, CASC4, FAM98B, NME1-NME2, HOOK3, CSTF3, ACTR3, RPL38, PLOD1, MARS, ZNF441, RELB, NLE1, MRPS23, and any combinations thereof.

39. The method of claim 25, wherein the biomarker is associated with an expression level of one or more genes selected from the group consisting of CALD1, SECISBP2L, UBXN8, AUP1, CDCA5, TM9SF3, LGR5, FAM98B, and combinations thereof.

40. The method of claim 25, wherein the biomarker is associated with an expression level of one or more genes selected from the group consisting of CALD1, SECISBP2L, UBXN8, AUP1, CDCA5, and any combinations thereof.

41. The method of claim 25, wherein the biomarker is associated with an expression level of one or more genes selected from the group consisting of CALD1, UBXN8, AUP1, CDCA5, and any combinations thereof.

42. The method of claim 25, wherein the biomarker is associated with an expression level of one or more genes selected from the group consisting of CALD1, SECISBP2L, UBXN8, AUP1, and any combinations thereof.

43. The method of claim 25, wherein the chemotherapy comprises a tubulin binding agent.

44. The method of claim 25, wherein determining the inhibition activity of the chemotherapy drug comprises measuring the inhibition activity after treating the cancer cell lines with a media containing the chemotherapy drug.

45. The method of claim 44, comprising treating the cancer cell lines with the media containing the chemotherapy drug for about 12 hours to 36 hours followed by treating the cancer cell lines with a media without the chemotherapy drug for about 48 hours to about 96 hours prior to measuring the inhibition activity.

46. The method of claim 44 or 45, comprising setting a threshold inhibition activity and assigning the inhibition activity of the chemotherapy drug on the plurality of cancer cell lines as active or inactive based on the threshold inhibition activity.

47. The method of claim 44, wherein the inhibition activity is based on an IC50, IC60, IC70, IC80, or IC90 value.

48. The method of any one of claims 25 to 45, further comprising classifying the inhibition activity and the ordinal activity status of the model or cell line as active or inactive based on the measured IC50, IC60, IC70, IC80, or IC90 value after comparing with a threshold value.
